SEQUENCE LISTING (37 sequences)

SEQ ID NO: 1 - DNA, 228 bp, Saccharomyces cerevisiae
cccctctgtatacttttcaactctgtgaagccgcaatttaaattaccggtaatagcatctgacgatttgaaagtctgtaaaaaatccatttatgactttatattgggctgcaagaaacactttgcatttaacgatgaggagcttttcactatatccgacgtttttgccaactcgacgtcccagctggtcaaagtgctagaagtagtagaaacgctaatgaattccagc

SEQ ID NO: 2 - protein, 76 aa, Saccharomyces cerevisiae
PLCILFNSVKPQFKLPVIASDDLKVCKKSIYDFILGCKKHFAFNDEELFTISDVFANSTSQLVKVLEVVETLMNSS

SEQ ID NO: 3 - DNA, 228 bp, Artificial Sequence
  cdc24-m1
cccctctgtatacttttcaactctgtgaagccgcaatttaaattaccggtaatagcatttgacgatttgaaagtctgtaaaaaatccatttatgactttatattgggctgcaagaaacactttgcatttaacgatgaggagcttttcactatatccgacgtttttgccaactcgacgtcccagctggtcaaagtgctagaagtagtagaaacgctaatgaattccagc

SEQ ID NO: 4 - protein, 76 aa, Artificial Sequence
  cdc24-m1
PLCILFNSVKPQFKLPVIAFDDLKVCKKSIYDFILGCKKHFAFNDEELFTISDVFANSTSQLVKVLEVVETLMNSS

SEQ ID NO: 5 - DNA, 228 bp, Artificial Sequence
  cdc24-m2
cccctctgtatacttttcaactctgtgaagccgcaatttaaattaccggtaatagcatctggcgatttgaaagtctgtaaaaaatccatttatgactttatattgggctgcaagaaacactttgcatttaacgatgaggagcttttcactatatccgacgtttttgccaactcgacgtcccagctggtcaaagtgctagaagtagtagaaacgctaatgaattccagc

SEQ ID NO: 6 - protein, 76 aa, Artificial Sequence
  cdc24-m2
PLCILFNSVKPQFKLPVIASGDLKVCKKSIYDFILGCKKHFAFNDEELFTISDVFANSTSQLVKVLEVVETLMNSS

SEQ ID NO: 7 - DNA, 228 bp, Artificial Sequence
  cdc24-m3
cccctctgtatacttttcaactctgtgaagccgcaatttaaattaccggtaatagcacctgacgatttgaaagtctgtaaaaaatccatttatgactttatattgggctgcaagaaacactttgcatttaacgatgaggagcttttcactatatccgacgtttttgccaactcgacgtcccagctggtcaaagtgctagaagtagtagaaacgctaatgaattccagc

SEQ ID NO: 8 - protein, 76 aa, Artificial Sequence
  cdc24-m3
PLCILFNSVKPQFKLPVIAPDDLKVCKKSIYDFILGCKKHFAFNDEELFTISDVFANSTSQLVKVLEVVETLMNSS

SEQ ID NO: 9 - protein, 392 aa, Artificial Sequence
  Yeast cdc24p DH PH
KIIKEFVATERKYVHDLEILDKYRQQLLDSNLITSEELYMLFPNLGDAIDFQRRFLISLEINALVEPSKQRIGALFMHSKHFFKLYEPWSIGQNAAIEFLSSTLHKMRVDESQRFIINNKLELQSFLYKPVQRLCRYPLLVKELLAESSDDNNTKELEAALDISKNIARSINENQRRTENHQVVKKLYGRVVNWKGYRISKFGELLYFDKVFISTTNSSSEPEREFEVYLFEKIIILFSEVVTKKSASSLILKKKSSTSASISASNITDNNGSPHHSYHKRHSNSSSSNNIHLSSSSAAAIIHSSTNSSDNNSNNSSSSSLFKLSANEPKLDLRGRIMIMNLNQIIPQNNRSLNITWESIKEQGNFLLKFKNEETRDNWSSCLQQLIHDLKN

SEQ ID NO: 10 - DNA, 1269 bp, Saccharomyces cerevisiae
atggcacatcagatggactcgataacgtattctaataatgtcacccaacagtatatacaaccacaaagtctacaggatatctctgcagtggaggaagaaattcaaaataaaatagaggccgccagacaagagagtaaacagcttcatgctcaaataaataaagcaaaacacaagatacaagatgcaagcttattccagatggccaacaaagttacttcgttgaccaaaaataagatcaacttaaagccaaatatcgtgttgaaaggccataataataaaatctcagattttcggtggagtcgagattcaaaacgtattttgagtgcaagtcaagatggctttatgcttatatgggacagtgcttcaggtttaaaacagaacgctattccattagattctcaatgggttctttcctgcgctatttcgccatcgagtactttggtagcaagcgcaggattaaacaataactgtaccatttatagagtttcgaaagaaaacagagtagcgcaaaacgttgcgtcaattttcaaaggacatacttgctatatttctgacattgaatttacagataacgcacatatattgacagcaagtggggatatgacatgtgccttgtgggatataccgaaagcaaagagggtgagagaatattctgaccatttaggtgatgttttggcattagctattcctgaagagccaaacttagaaaattcttcgaacacattcgctagctgtggatcagacgggtatacttacatatgggatagcagatctccgtccgctgtacaaagcttttacgttaacgatagtgatattaatgcacttcgttttttcaaagacgggatgtcgattgttgcaggaagtgacaatggtgcgataaatatgtatgatttaaggtcggactgttctattgctactttttctctttttcgaggttatgaagaacgtacccctacccctacttatatggcagctaacatggagtacaataccgcgcaatcgccacaaactttaaaatcaacaagctcaagctatctagacaaccaaggcgttgtttctttagattttagtgcatctggaagattgatgtactcatgctatacagacattggttgtgttgtgtgggatgtattaaaaggagagattgttggaaaattagaaggtcatggtggcagagtcactggtgtgcgctcgagtccagatgggttagctgtatgtacaggttcatgggactcaaccatgaaaatatggtctccaggttatcaatag

SEQ ID NO: 11 - protein, 422 aa, Saccharomyces cerevisiae
MAHQMDSITYSNNVTQQYIQPQSLQDISAVEEEIQNKIEAARQESKQLHAQINKAKHKIQDASLFQMANKVTSLTKNKINLKPNIVLKGHNNKISDFRWSRDSKRILSASQDGFMLIWDSASGLKQNAIPLDSQWVLSCAISPSSTLVASAGLNNNCTIYRVSKENRVAQNVASIFKGHTCYISDIEFTDNAHILTASGDMTCALWDIPKAKRVREYSDHLGDVLALAIPEEPNLENSSNTFASCGSDGYTYIWDSRSPSAVQSFYVNDSDINALRFFKDGMSIVAGSDNGAINMYDLRSDCSIATFSLFRGYEERTPTPTYMAANMEYNTAQSPQTLKSTSSSYLDNQGVVSLDFSASGRLMYSCYTDIGCVVWDVLKGEIVGKLEGHGGRVTGVRSSPDGLAVCTGSWDSTMKIWSPGYQ

SEQ ID NO: 12 - DNA, 1269 bp, Artificial Sequence
  ste4-o15
atggcacatcagatggactcgataacgtattctaataatgtcacccaacagtatatacaaccacaaagtctacaggatatctctgcagtggaggaagaaattcaaaataaaatagaggccgccagacaagagagtaaacagcttcatgctcaaataaataaagcaaaacacaagatacaagatgcaagcttattccagatggccaacaaagttacttcgttgaccaaaaataagatcaacttaaagccaaatatcgtgttgaaaggccataataataaaatctcagattttcggtggagtcgagattcaaaacgtattttgagtgcaagtcaagatggctttatgcttatatgggacagtgcttcaggtttaaaacagaacgctattccattagattctcaatgggttctttcctgcgctatttcgccatcgagtactttggtagcaagcgcaggattaaacaataactgtaccatttatagagtttcgaaagaaaacagagtagcgcaaaacgttgcgtcaattttcaaaggacatacttgctatatttctgacattgaatttacagataacgcacatatattgacagcaagtggggatatgacatgtgccttgtgggatataccgaaagcaaagagggtgagaggatattctgaccatttaggtgatgttttggcattagctattcctgaagagccaaacttagaaaattcttcgaacacattcgctagctgtggatcagacgggtatacttacatatgggatagcagatctccgtccgctgtacaaagcttttacgttaacgatagtgatattaatgcacttcgttttttcaaagacgggatgtcgattgttgcaggaagtgacaatggtgcgataaatatgtatgatttaaggtcggactgttctattgctactttttctctttttcgaggttatgaagaacgtacccctacccctacttatatggcagctaacatggagtacaataccgcgcaatcgccacaaactttaaaatcaacaagctcaagctatctagacaaccaaggcgttgtttctttagattttagtgcatctggaagattgatgtactcatgctatacagacattggttgtgttgtgtgggatgtattaaaaggagagattgttggaaaattagaaggtcatggtggcagagtcactggtgtgcgctcgagtccagatgggttagctgtatgtacaggttcatgggactcaaccatgaaaatatggtctccaggttatcaatag

SEQ ID NO: 13 - protein, 422 aa, Artificial Sequence
  ste4-o15
MAHQMDSITYSNNVTQQYIQPQSLQDISAVEEEIQNKIEAARQESKQLHAQINKAKHKIQDASLFQMANKVTSLTKNKINLKPNIVLKGHNNKISDFRWSRDSKRILSASQDGFMLIWDSASGLKQNAIPLDSQWVLSCAISPSSTLVASAGLNNNCTIYRVSKENRVAQNVASIFKGHTCYISDIEFTDNAHILTASGDMTCALWDIPKAKRVRGYSDHLGDVLALAIPEEPNLENSSNTFASCGSDGYTYIWDSRSPSAVQSFYVNDSDINALRFFKDGMSIVAGSDNGAINMYDLRSDCSIATFSLFRGYEERTPTPTYMAANMEYNTAQSPQTLKSTSSSYLDNQGVVSLDFSASGRLMYSCYTDIGCVVWDVLKGEIVGKLEGHGGRVTGVRSSPDGLAVCTGSWDSTMKIWSPGYQ

SEQ ID NO: 14 - DNA, 1269 bp, Artificial Sequence
  ste4-o17
atggcacatcagatggactcgataacgtattctaataatgtcacccaacagtatatacaaccacaaagtctacaggatatctctgcagtggaggaagaaattcaaaataaaatagaggccgccagacaagagagtaaacagcttcatgctcaaataaataaagcaaaacacaagatacaagatgcaagcttattccagatggccaacaaagttacttcgttgaccaaaaataagatcaacttaaagccaaatatcgtgttgaaaggccataataataaaatctcagattttcggtggagtcgagattcaaaacgtattttgagtgcaagtcaagatggctttatgcttatatgggacagtgcttcaggtttaaaacagaacgctattccattagattctcaatgggttctttcctgcgctatttcgccatcgagtactttggtagcaagcgcaggattaaacaataactgtaccatttatagagtttcgaaagaaaacagagtagcgcaaaacgttgcgtcaattttcaaaggacatacttgctatatttctgacattgaatttacagataacgcacatatattgacagcaagtggggatatgacatgtgccttgtgggatataccgaaagcaaagagggtgagagaatattctgaccatttaggtgatgttttggcattagctattcctgaagagccaaacttagaaaattcttcgaacacattcgctagctgtggatcagacgggtatacttacatatgggatagcagatctccgtccgctgtacaaagcttttacgttaacgatagtgatattaatgcacttcgttttttcaaagacgggatgtcgattgttgcaggaagtgacaatggtgcgataaatatgtatgatttaaggtcggactgttctattgctactttttctctttttcgaggttatgaagaacgtacccctacccctacttatatggcagctaacatggagtacaataccgcgcaatcgccacaaactttaaaatcaacaagctcaagctatctagacaaccaaggcgctgtttctttagattttagtgcatctggaagattgatgtactcatgctatacagacattggttgtgttgtgtgggatgtattaaaaggagagattgttggaaaattagaaggtcatggtggcagagtcactggtgtgcgctcgagtccagatgggttagctgtatgtacaggttcatgggactcaaccatgaaaatatggtctccaggttatcaatag

SEQ ID NO: 15 - protein, 422 aa, Artificial Sequence
  ste4-o17
MAHQMDSITYSNNVTQQYIQPQSLQDISAVEEEIQNKIEAARQESKQLHAQINKAKHKIQDASLFQMANKVTSLTKNKINLKPNIVLKGHNNKISDFRWSRDSKRILSASQDGFMLIWDSASGLKQNAIPLDSQWVLSCAISPSSTLVASAGLNNNCTIYRVSKENRVAQNVASIFKGHTCYISDIEFTDNAHILTASGDMTCALWDIPKAKRVREYSDHLGDVLALAIPEEPNLENSSNTFASCGSDGYTYIWDSRSPSAVQSFYVNDSDINALRFFKDGMSIVAGSDNGAINMYDLRSDCSIATFSLFRGYEERTPTPTYMAANMEYNTAQSPQTLKSTSSSYLDNQGAVSLDFSASGRLMYSCYTDIGCVVWDVLKGEIVGKLEGHGGRVTGVRSSPDGLAVCTGSWDSTMKIWSPGYQ

SEQ ID NO: 16 - protein, 9 aa, Artificial Sequence
  Epitope sequence
YPYDVPDYA

SEQ ID NO: 17 - protein, 7 aa, Artificial Sequence
  TEV protease recognition sequence
QNLYFQG

SEQ ID NO: 18 - protein, 19 aa, Artificial Sequence
  S. cerevisiae Cdc24-m1
QFKLPVIAFDDLKVCKKSI

SEQ ID NO: 19 - protein, 19 aa, Artificial Sequence
  S. cerevisiae Cdc24-m2
QFKLPVIASGDLKVCKKSI

SEQ ID NO: 20 - protein, 19 aa, Artificial Sequence
  S. cervisiae Cdc24-m3
QFKLPVIAPDDLKVCKKSI

SEQ ID NO: 21 - protein, 19 aa, Saccharomyces cerevisiae
QFKLPVIASDDLKVCKKSI

SEQ ID NO: 22 - protein, 19 aa, Homo sapien
QYEFDVILSPELKVQMKTI

SEQ ID NO: 23 - DNA, 2535 bp, Candida albicans
atggaacatccaccagcagctctcagaacattttcaacccaatcaacttcatctttgaattcagtaagtactgtttcgtcttcaagaattgtttctctgggcccagtcaatataaacaatttcaataaaccaagtactcccaaagaccatttattctatcgatgtgaatcactaaaacgaaaactacaaaaaatccctggcatggaaccatttttgaaccaagctttcaatcaggctgaacaactcagtgaacaacaagcattggctttggcacaggaaagaagcaatggaaatggacatagtaatggcaaacgtcatcaatcattagacggtgccatgaatagactttcagttggttctgatagtagttcgatccaaggttcattgacacgaatggccaccaatgcgtcaacgtcatctttaatcagtggtatgccaaacaacaacactttatttacgtttactgcaggggttttaccagctaatattagtgtcgatcctgctacccatctttggaaattgttccaacaaggggcccccttttgtgttcttatcaatcatatccttcctgattcccaaataccagttgtcagttctgatgacttgagaatttgcaaaaaatcagtatatgactttttaattgccgtcaagacacaattgaattttgatgacgagaatatgttcactatatccaatgttttctccgacaatgcccaagatttaatcaagattattgatgtcattaataaactacttgctgagtactcagatgctagtgacctgggtggtggcgatgaagatgtaaatatggatgttcaaattaccgatgaaagatcaaaagttttccgagaaattatcgaaacagaaagaaaatatgttcaagacttggaactaatgtgtaaataccgtcaagatctaattgaagccgaaaatttgtcttcagaacaaattcacttgttattcccaaatttaaatgagattattgattttcaaagacgattcctcaatgggttagaatgtaacatcaatgtacctattagatatcaaagaattggatcagtatttattcatgcttctttgggccctttcaatgcttatgaaccttggactataggacaattgacggcgattgatttgatcaacaaagaagctgctaatttgaaaaaatcgtcaagtctacttgatcctgggtttgaacttcaatcgtatatattaaagccgatccaaagattgtgtaaatacccacttttgttgaaagagttaatcaaaacatcaccagaatattcaaaacaggacccccatggcagctcgtcatcgacatcattcaatgaattattggtggctaaaactgcaatgaaagaattggcaaatcaagtcaatgaggcgcaaagacgagcagaaaatatcgaacatttggaaaaactaaaagaaagagtaggtaattggcgtgggtttaatttggatgctcaaggagaactattattccacggacaagttggggttaaagatgctgaaaatgaaaaggaatacgttgcttatctttttgaaaaaatcgtattttttttcacagaaattgatgataccaaaaaatctgataaacaggaaaagaagagcaagttttcgacaagaaagagatcaacttcatcaaatcttagttcatcgactactaatttgttggaatcaataaacaattcccgaaaggataacacattgccattggaattaaagggaagagtttatatatcggagatttataacatttccgcaccaaacactcctggctcaactctaatcatctcatggtcaggtagaaaggaaagcggctcattcactttgagatatcgtagtgaagaagccagaaaccaatgggaaaagtgtttacgtgatttgaagactaatgaaatgaataaacaaattcataagaagttacgtgattccgacctgtcatttaatactgatgactctgccatatatgattacacgggtattagtacgtcaccagtcaatcaatcaactcaacaacaatactatgatcatcggggctctcacagttcccgccatcactcatcgtcatccactttgagtatgatgaagaataatagagttaaatctggtgatttgagtagaatatcttcaacttcaacaacattagattctttcagtaacaacttgaatgggtcaccaaataccactaatccatctttgatgtcttcagatgccaccaaaacaattccaacatttgacgttgcaattaaattgctttacaaatcgacagaattgtcagagccattgattgtcaatgcacaaattgagtataatgaccttttacagaaaattatctcccagattatcacttcgaacttggtggcagatgatgtcaatattagtcgattgagatataaagacgacgaaggagactttgtgaatttgaattcagatgatgattgggggttagtgcttgatatgttaaccagtgaagacttttaccaaacatcaagcaatgaaaaacgactggtgacagtgtgggtttcttga

SEQ ID NO: 24 - protein, 844 aa, Candida albicans
MEHPPAALRTFSTQSTSSLNSVSTVSSSRIVSSGPVNINNFNKPSTPKDHLFYRCESLKRKLQKIPGMEPFLNQAFNQAEQLSEQQALALAQERSNGNGHSNGKRHQSLDGAMNRLSVGSDSSSIQGSLTRMATNASTSSLISGMPNNNTLFTFTAGVLPANISVDPATHLWKLFQQGAPFCVLINHILPDSQIPVVSSDDLRICKKSVYDFLIAVKTQLNFDDENMFTISNVFSDNAQDLIKIIDVINKLLAEYSDASDSGGGDEDVNMDVQITDERSKVFREIIETERKYVQDLELMCKYRQDLIEAENLSSEQIHLLFPNLNEIIDFQRRFLNGLECNINVPIRYQRIGSVFIHASLGPFNAYEPWTIGQLTAIDLINKEAANLKKSSSLLDPGFELQSYILKPIQRLCKYPLLLKELIKTSPEYSKQDPHGSSSSTSFNELLVAKTAMKELANQVNEAQRRAENIEHLEKLKERVGNWRGFNLDAQGELLFHGQVGVKDAENEKEYVAYLFEKIVFFFTEIDDTKKSDKQEKKSKFSTRKRSTSSNLSSSTTNLLESINNSRKDNTLPLELKGRVYISEIYNISAPNTPGSTLIISWSGRKESGSFTLRYRSEEARNQWEKCLRDLKTNEMNKQIHKKLRDSDSSFNTDDSAIYDYTGISTSPVNQSTQQQYYDHRGSHSSRHHSSSSTLSMMKNNRVKSGDLSRISSTSTTLDSFSNNLNGSPNTTNPSLMSSDATKTIPTFDVAIKLLYKSTELSEPLIVNAQIEYNDLLQKIISQIITSNLVADDVNISRLRYKDDEGDFVNLNSDDDWGLVLDMLTSEDFYQTSSNEKRSVTVWVS

SEQ ID NO: 25 - protein, 22 aa, Saccharomyces cerevisiae
KLPVIASDDLKVCKKSIYDFIL

SEQ ID NO: 26 - protein, 22 aa, Candida albicans
QIPVVSSDDLRICKKSVYDFLI

SEQ ID NO: 27 - protein, 854 aa, Saccharomyces cerevisiae
MAIQTRFASGTSLSDLKPKPSATSISIPMQNVMNKPVTEQDSLFHICANIRKRLEVLPQLKPFLQLAYQSSEVLSERQSLLLSQKQHQELLKSNGANRDSSDLAPTLRSSSISTATSLMSMEGISYTNSNPSATPNMEDTLLTFSMGILPITMDCDPVTQLSQLFQQGAPLCILFNSVKPQFKLPVIASDDLKVCKKSIYDFILGCKKHFAFNDEELFTISDVFANSTSQLVKVLEVVETLMNSSPTIFPSKSKTQQIMNAENQHRHQPQQSSKKHNEYVKIIKEFVATERKYVHDLEILDKYRQQLLDSNLITSEELYMLFPNLGDAIDFQRRFLISLEINALVEPSKQRIGALFMHSKHFFKLYEPWSIGQNAAIEFLSSTLHKMRVDESQRFIINNKLELQSFLYKPVQRLCRYPLLVKELLAESSDDNNTKELEAALDISKNIARSINENQRRTENHQVVKKLYGRVVNWKGYRISKFGELLYFDKVFISTTNSSSEPEREFEVYLFEKIIILFSEVVTKKSASSLILKKKSSTSASISASNITDNNGSPHHSYHKRHSNSSSSNNIHLSSSSAAAIIHSSTNSSDNNSNNSSSSSLFKLSANEPKLDLRGRIMIMNLNQIIPQNNRSLNITWESIKEQGNFLLKFKNEETRDNWSSCLQQLIHDLKNEQFKARHHSSTSTTSSTAKSSSMMSPTTTMNTPNHHNSRQTHDSMASFSSSHMKRVSDVLPKRRTTSSSFESEIKSISENFKNSIPESSILFRISYNNNSNNTSSSEIFTLLVEKVWNFDDLIMAINSKISNTHNNNISPITKIKYQDEDGDFVVLGSDEDWNVAKEMLAENNEKFLNIRLY

SEQ ID NO: 28 - protein, 837 aa, Saccharomyces cerevisiae
SGTSLSDLKPKPSATSISIPMQNVMNKPVTEQDSLFHICANIRKRLEVLPQLKPFLQLAYQSSEVLSERQSLLLSQKQHQELLKSNGANRDSSDLAPTLRSSSISTATSLMSMEGISYTNSNPSATPNMEDTLLTFSMGILPITMDCDPVTQLSQLFQQGAPLCILFNSVKPQFKLPVIASDDLKVCKKSIYDFILGCKKHFAFNDEELFTISDVFANSTSQLVKVLEVVETLMNSSPTIFPSKSKTQQIMNAENQHRHQPQQSSKKHNEYVKIIKEFVATERKYVHDLEILDKYRQQLLDSNLITSEELYMLFPNLGDAIDFQRRFLISLEINALVEPSKQRIGALFMHSKHFFKLYEPWSIGQNAAIEFLSSTLHKMRVDESQRFIINNKLELQSFLYKPVQRLCRYPLLVKELLAESSDDNNTKELEAALDISKNIARSINENQRRTENHQVVKKLYGRVVNWKGYRISKFGELLYFDKVFISTTNSSSEPEREFEVYLFEKIIILFSEVVTKKSASSLILKKKSSTSASISASNITDNNGSPHHSYHKRHSNSSSSNNIHLSSSSAAAIIHSSTNSSDNNSNNSSSSSLFKLSANEPKLDLRGRIMIMNLNQIIPQNNRSLNITWESIKEQGNFLLKFKNEETRDNWSSCLQQLIHDLKNEQFKARHHSSTSTTSSTAKSSSMMSPTTTMNTPNHHNSRQTHDSMASFSSSHMKRVSDVLPKRRTTSSSFESEIKSISENFKNSIPESSILFRISYNNNSNNTSSSEIFTLLVEKVWNFDDLIMAINSKISNTHNNNISPITKIKYQDEDGDFVVLGSDEDWNVAKEMLAENN

SEQ ID NO: 29 - protein, 813 aa, Candida albicans
STSSLNSVSTVSSSRIVSSGPVNINNFNKPSTPKDHLFYRCESLKRKLQKIPGMEPFLNQAFNQAEQLSEQQALALAQERSNGNGHSNGKRHQSLDGAMNRLSVGSDSSSIQGSLTRMATNASTSSLISGMPNNNTLFTFTAGVLPANISVDPATHLWKLFQQGAPFCVLINHILPDSQIPVVSSDDLRICKKSVYDFLIAVKTQLNFDDENMFTISNVFSDNAQDLIKIIDVINKLLAEYSDASDSGGGDEDVNMDVQITDERSKVFREIIETERKYVQDLELMCKYRQDLIEAENLSSEQIHLLFPNLNEIIDFQRRFLNGLECNINVPIRYQRIGSVFIHASLGPFNAYEPWTIGQLTAIDLINKEAANLKKSSSLLDPGFELQSYILKPIQRLCKYPLLLKELIKTSPEYSKQDPHGSSSSTSFNELLVAKTAMKELANQVNEAQRRAENIEHLEKLKERVGNWRGFNLDAQGELLFHGQVGVKDAENEKEYVAYLFEKIVFFFTEIDDTKKSDKQEKKSKFSTRKRSTSSNLSSSTTNLLESINNSRKDNTLPLELKGRVYISEIYNISAPNTPGSTLIISWSGRKESGSFTLRYRSEEARNQWEKCLRDLKTNEMNKQIHKKLRDSDSSFNTDDSAIYDYTGISTSPVNQSTQQQYYDHRGSHSSRHHSSSSTLSMMKNNRVKSGDLSRISSTSTTLDSFSNNLNGSPNTTNPSLMSSDATKTIPTFDVAIKLLYKSTELSEPLIVNAQIEYNDLLQKIISQIITSNLVADDVNISRLRYKDDEGDFVNLNSDDDWGLVLDMLTSED

SEQ ID NO: 30 - protein, 684 aa, Saccharomyces cerevisiae
DPVTQLSQLFQQGAPLCILFNSVKPQFKLPVIASDDLKVCKKSIYDFILGCKKHFAFNDEELFTISDVFANSTSQLVKVLEVVETLMNSSPTIFPSKSKTQQIMNAENQHRHQPQQSSKKHNEYVKIIKEFVATERKYVHDLEILDKYRQQLLDSNLITSEELYMLFPNLGDAIDFQRRFLISLEINALVEPSKQRIGALFMHSKHFFKLYEPWSIGQNAAIEFLSSTLHKMRVDESQRFIINNKLELQSFLYKPVQRLCRYPLLVKELLAESSDDNNTKELEAALDISKNIARSINENQRRTENHQVVKKLYGRVVNWKGYRISKFGELLYFDKVFISTTNSSSEPEREFEVYLFEKIIILFSEVVTKKSASSLILKKKSSTSASISASNITDNNGSPHHSYHKRHSNSSSSNNIHLSSSSAAAIIHSSTNSSDNNSNNSSSSSLFKLSANEPKLDLRGRIMIMNLNQIIPQNNRSLNITWESIKEQGNFLLKFKNEETRDNWSSCLQQLIHDLKNEQFKARHHSSTSTTSSTAKSSSMMSPTTTMNTPNHHNSRQTHDSMASFSSSHMKRVSDVLPKRRTTSSSFESEIKSISENFKNSIPESSILFRISYNNNSNNTSSSEIFTLLVEKVWNFDDLIMAINSKISNTHNNNISPITKIKYQDEDGDFVVLGSDEDWNVAKE

SEQ ID NO: 31 - protein, 742 aa, Schizosaccharomyces pombe
DPVTEIWLFCRLGYPLCALFNCLPVKQKLEVNSSVSLENTNVCKASLYRFMLMCKNELGLTDAALFSISEIYKPSTAPLVRALQTIELLLKKYEVSNTTKSSSTPSPSTDDNVPTGTLNSLIASGRRVTAELYETELKYIQDLEYLSNYMVILQQKQILSQDTILSIFTNLNEILDFQRRFLVGLEMNLSLPVEEQRLGALFIALEEGFSVYQVFCTNFPNAQQLIIDNQNQLLKVANLLEPSYELPALLIKPIQRICKYPLLLNQLLKGTPSGYQYEEELKQGMACVVRVANQVNETRRIHENRNAIIELEQRVIDWKGYSLQYFGQLLVWDVVNVCKADIEREYHVYLFEKILLCCKEMSTLKRQARSISMNKKTKRLDSLQLKGRILTSNITTVVPNHHMGSYAIQIFWRGDPQHESFILKLRNEESHKLWMSVLNRLLWKNEHGSPKDIRSAASTPANPVYNRSSSQTSKGYNSSDYDLLRTHSLDENVNSPTSISSPSSKSSPFTKTTSKDTKSATTTDERPSDFIRLNSEESVGTSSLRTSQTTSTIVSNDSSSTASIPSQISRISQVNSLLNDYNYNRQSHITRVYSGTDDGSSVSIFEDTSSSTKQKIFDQPTTNDCDVMRPRQYSYSAGMKSDGSLLPSTKHTSLSSSSTSTSLSVRNTTNVKIRLRLHEVSLVLVVAHDITFDELLAKVEHKIKLCGILKQAVPFRVRLKYVDEDGDFITITSDEDVLMAFE

SEQ ID NO: 32 - DNA, 20 bp, Artificial Sequence
  Primer
aartayrtkcangayttrga

SEQ ID NO: 33 - DNA, 18 bp, Artificial Sequence
  Primer
rattttytcraanarrta

SEQ ID NO: 34 - protein, 76 aa, Candida albicans
PFCVLINHILPDSQIPVVSSDDLRICKKSVYDFLIAVKTQLNFDDENMFTISNVFSDNAQDLIKIIDVINKLLAEY

SEQ ID NO: 35 - protein, 19 aa, Candida albicans
DSQIPVVSSDDLRICKKSV

SEQ ID NO: 36 - protein, 73 aa, Candida albicans
PFCVLINHILPDSQIPVVSSDDLRICKKSVYDFLIAVKTQLNFDDENMFTISNVFSDNAQDLIKIIDVINKLL

SEQ ID NO: 37 - protein, 73 aa, Saccharomyces cerevisiae
PLCILFNSVKPQFKLPVIASDDLKVCKKSIYDFILGCKKHFAFNDEELFTISDVFANSTSQLVKVLEVVETLM

(19) United States
(12) Patent Application Publication
(10) Pub. No.: US 2003/0175712 A1
(43) Pub. Date: Sep. 18, 2003

(54) Nucleotide sequences and protein sequences

(76) Inventors: Peter Michael Aljoscha Nern, Los Angeles, CA (US); Robert A. Arkowitz, Nice Cedex (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/054,399

(22) Filed: Jan. 21, 2002

Related U.S. Application Data
(63) Continuation-in-part of application No. 09/168,474, filed on Oct. 8, 1998, now abandoned.
(63) Continuation-in-part of application No. 09/732,180, filed on Dec. 7, 2000, now abandoned.
(60) Provisional application No. 60/169,699, filed on Dec. 7, 1999.

(30) Foreign Application Priority Data
Oct. 8, 1997 (GB) GB9721358.1
Oct. 8, 1997 (GB) GB9721357.3
Jun. 12, 1998 (GB) GB9812793.9

(51) Int. Cl. C12Q 1/68; C07H 21/04; A61K 48/00; C07K 14/40

(52) U.S. Cl. 435/6; 514/44; 536/23.2; 530/350

(57) ABSTRACT

A nucleotide sequence is described. The nucleotide sequence or the expression product of the nucleotide sequence has the capability of not substantially affecting the interaction of Gβ with Cdc24p or a homologue thereof that is usually capable of being associated with the Cdc24p or the homologue thereof.

### RELATED APPLICATIONS

This application is a continuation-in-part of U.S. application Ser. No. 09/168,474 filed Oct. 8, 1998, which claims the benefit of priority under 35 U.S.C. §119(e) of U.S. application Ser. No. 08/951,141, which was filed as a nonprovisional application on Oct. 15, 1997 and converted to a provisional application by petition mailed by Applicants on Oct. 8, 1998, and also claims the benefit of priority under 35 U.S.C. §119(a) to applications GB9721358.1 filed Oct. 8, 1997, GB9721357.3 filed Oct. 8, 1997, and GB9812793.9 filed Jun. 12, 1998. This application is also a continuation-in-part of U.S. application Ser. No. 09/732,180 filed Dec. 7, 2000, which claims the benefit of priority to U.S. Provisional Application No. 60/169,699 filed Dec. 7, 1999. The complete disclosures of the above-referenced related applications are herein incorporated by reference.

### FIELD OF THE INVENTION

The present invention relates to nucleotide sequences and protein sequences. In particular, the present invention relates to nucleotide sequences and protein sequences that affect interactions of cellular components.

### BACKGROUND TO THE INVENTION

According to Cerione and Zheng (The Dbl family of oncogenes Current Opinion In Cell Biology 8, 216-222 (1996)), genetic screening and biochemical studies during the past years have led to the discovery of a certain family of cell growth regulatory proteins and oncogene products for which the Dbl oncoprotcin is the prototype. Another review on Dbl is presented by Machesky and Hall (1996 Trends In Cell Biology 6 pp 3-4-310).

Cerione and Zheng (ibid say that proto-Dbl is a 115 kDa cytoskeleton-associated protein that is found in tissues such as brain, ovary, testis and adrenal glands. Oncogenic activation of proto-Dbl occurs as a result of an amino-terminal truncation of proto-Dbl which leaves residues 498-925 fused with the product of an as yet unidentified gene which is localised on chromosome 3.

Cerione and Zheng also say that a region located between residues 498 and 674 of proto-Dbl—which is retained by oncogenic Dbl—has significant similarities with the Saccharomyces cerevisiae cell division cycle molecule Cdc24p and the breakpoint cluster gene product Bcr (see also Hart et al 1991 Nature 354 311-314; Miyamoto et al 1991 Biochem Biophys Res Commun 181 604-610; Ron et al 1991 New Biol 3 372-379). This region—which is referred to as being the DH domain—was later shown to be responsible for the GEF (GDP-GTP Exchange factor—otherwise known as a guanine nucleotide exchange factor) activity of the Dbl oncoprotein and to be critical for its transforming function (see also Hart et al J Biol Chem 269 62-65).

Cerione and Zheng also report that since the initial identification of Dbl as a GEF for Rho-type GTP binding proteins, a number of oncogene products and growth regulatory molecules have been shown to contain a DH domain in tandem with another region designated PH (i.e. a pleckstrin homology domain which is found between residues 703-812 in of proto-Dbl). Many of these products and molecules, such as Bcr, Cdc24, Sos, Vav, ect-2, Ost, Tim, Lbc, Lfc and Dbc, form a family of GEfs which have been implicated in cell growth regulation. Cerione and Zheng provide details on each of these products and molecules. In addition, these and other products and molecules are discussed below.

Cerione and Zheng (ibid) end their Abstract by saying:

“Despite the increasing interest in the Dbl family of proteins, there is still a good deal to learn regarding the biochemical mechanisms that underlie their diverse biological functions.”

As mentioned above, it is known that proto-Dbl has significant similarities with the S. cerevisiae cell division cycle molecule Cdc24p which is a GEF for the Rho-family GTPase molecule Cdc42p (see again Hart et al 1991 Nature 354 311-314; Miyamoto et al 1991 Biochem Biophys Res Commun 181 604-610; Ron et al 1991 New Biol 3 372-379; Zheng et al 1994 J Biol Chem 269 2369-2372). However, whilst it is known that the Rho-family GTPascs and their regulators are essential for cytoskeletal reorganisation and transcriptional activation in response to extracellular signals¹², little is known about what links these molecules to membrane receptors. For example, in the budding yeast S. cerevisiae, haploid cells respond to mating pheromone through a G-protein coupled receptor (Ste2p/Ste3p) via Gβγ (Ste4p/Ste18p) resulting in cell cycle arrest, transcriptional activation, and polarised growth towards a mating partner^{4,5}. Recently, the Rho-family GTPase Cdc42p and its exchange factor Cdc24p have been implicated in the mating process^{6,7}but their specific role is unknown.

### SUMMARY OF THE INVENTION

In our studies (which are presented below) on S. cerevisiae we have been able to identify hitherto unrecognised regions that play a key role in the interaction of cellular components. This finding has broad implications—not only for the design of anti-fungal drugs (such as those that could be directed against the yeast Candida) but also in the screening and design of agents that can affect oncogenes such as Dbl, in partcular proto-Dbl.

Moreover, in our studies (which are presented below), we have identified novel cdc24 alleles which do not affect vegetative growth but drastically reduce the ability of yeast cells to mate. When exposed to mating pheromone these mutants arrest growth, activate transcription, and undergo characteristic morphological and actin cytoskeleton polarisation. However, the mutants are unable to orient towards a pheromone gradient and instead position their mating projection adjacent to their previous bud site. Strikingly, these mutants are specifically defective in the binding of Cdc24p to Gβγ. This work demonstrates that the association of a GEF and the βγ-subunit of a hetero-trimeric G-protein (Gβγ) links receptor-mediated activation to oriented cell growth.

The present invention also demonstrates that Far1, a cyclic dependent kinase inhibitor (CDK1) may also be implicated as being important for orientated cell growth.

Thus, according to one broad aspect of the present invention there is provided a GEF capable of interacting with a Gβ such that the interaction provides a connection between G protein coupled receptor activation and polarised cell growth.

According to another broad aspect of the present invention there is also provided an agent capable of affecting a GEF/Gβ interaction, which interaction provides a connection between G protein coupled receptor activation and polarised cell growth.

These and other aspects of the present invention are set out in thc following numnbered paragraphs.

1. A nucleotide sequence shown as SEQ I.D. No: 1, or a derivative, fragment variant or homologue of the nucleotide sequence wherein the expression product of the nucleotide sequence has the capability of not substantially affecting the interaction of Gβ with Cdc24p or a homologue thereof that is usually capable of being associated therewith.

2. A derivative, fragment, variant or homologue of the nucleotide sequence shown as SEQ I.D. No: 1.

3. A homologue according to paragraph 2 wherein the homologue comprises nucleotide residues 508 to 735 of the C.albicans Cdc24 gene presented as SEQ. I.D. No: 23.

4. A mutant of the nucleotide sequence shown as SEQ I.D. No: 1 or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

5. A method of medical treatment comprising the step of administering a nucleotide sequence shown as SEQ I.D. No: 1 or a derivative, fragment, variant or homologue thereof.

6. A method of medical treatment according to paragraph 5 wherein the homologue comprises nucleotide residues 508 to 735 of the C.albicans Cdc24 gene presented as SEQ. I.D. No: 23.

7. A method of medical treatment comprising the step of administering a mutant of the nucleotide sequence shown as SEQ I.D. No: 1 or a derivative, fragment, variant or homologue thereof or the expression product thereof.

8. A method of affecting the growth behaviour of cells comprising the step of administering the nucleotide sequence shown as SEQ I.D. No: 1 or a derivative, fragment, variant or homologue thereof or the expression product thereof to the cells.

9. A method of affecting te growth behaviour of cells according to paragraph 8, wherein the homologue comprises nucleotide residues 508 to 735 of the C.albicans Cdc24 gene presented as SEQ. I.D. No: 23.

10. A method of affecting the growth behaviour of cells comprising the step of administering a mutant of the nucleotide sequence shown as SEQ I.D. No: 1 or a derivative, fragment, variant or homologue thereof or the expression product thereof to the cells.

11. Use of a nucleotide sequence shown as SEQ I.D. No: 1 or a derivative, fragment, variant or homologue thereof or the expression product thereof in a screen to identify one or more agents that are capable of affecting the interaction of Cdc24p or a homologue thereof with a Gβ or an associated Rho-family GTPase.

12. The use according to paragraph 11, wherein the homologue comprises nucleotide residues 508 to 735 of the C.albicans Cdc24 gene presented as SEQ I.D. No 23.

13. Use of a mutant of a nucleotide sequence shown as SEQ I.D. No: 1 or a derivative, fragment, variant or homologue thereof or the expression product thereof in a screen to identify one or more agents that are capable of affecting the interaction of Cdc24p or a homologue thereof with a Gβ or an associated Rho-family GTPase.

14. An assay comprising contacting an agent with a nucleotide sequence shown as SEQ I.D. No: 1 or a derivative, fragment, variant or homologue thereof or the expression product thereof in the presence of a Gβ capable of being associated with Cdc24p or a homolope thereof, and determining whether the agent is capable of affecting the interaction of the nucleotide sequence or the expression product with the Gβ.

15. An assay according to paragraph 14 wherein the homologue comprises nucleotide residues 508 to 735 of the C.albicans Cdc24 gene presented as SEQ. I.D. No: 23.

16. An assay comprising contacting an agent with a mutant of a nucleotide sequence shown as SEQ I.D. No: 1 or a derivative, fragment, variant or homologue thereof or the expression product thereof in the presence of a Gβ capable of being associated with Cdc24p or a homologue thereof; and determining whether the agent is capable of affecting the interaction of the mutant nucleotide sequence or the expression product with the Gβ.

17. A kit comprising a nucleotide sequence shown as SEQ. I.D. No: 1 or a derivative, fagment, variant or homologue thereof or the expression product thereof; and a Gβ capable of being associated with Cdc24p or a homologue thereof.

18. A kit according to paragraph 17 comprising a homologue of SEQ I.D. No 1, wherein the homologue comprises nucleotide residues 508 to 735 of the C.albicans Cdc24 gene presented as SEQ. I.D. No: 23.

19. A kit comprising a mutant of a nucleotide sequence shown as SEQ I.D. No: 1 or a derivative, fragment, variant or homologue thereof or the expression product thereof; and a Gβ capable of being associated with Cdc24p or a homologue thereof.

20. A protein sequence shown as SEQ I.D. No: 2 or a derivative, fragment, variant or homologue thereof, wherein the protein has the capability of not substantially affecting the interaction of Gβ with Cdc24p or a homologue thereof that is usually capable of being associated with the Cdc24p or the homologue thereof.

21. A fragment of the protein sequence shown as SEQ. I.D. No: 2 according to paragraph 19 wherein the fragment is the 19 amino acid Cdc24 fragment SEQ. I.D. No: 21 or the 19 amino acid Dbl fragment SEQ. I.D. No 22.

22. A homologue of the protein sequence according to paragraph 20, wherein the homologue is the C.albicans Cdc24 76 amino acid fragment SEQ. I.D. No: 34.

23. A mutant of the protein sequence shown as SEQ I.D. No: 2 or a derivative, fragment, variant or homologue thereof, wherein the mutant protein has the capability of substantially affecting the interaction of Gβ with Cdc24p or a homologue thereof that is usually capable of being associated with the Cdc24p or the homologue thereof.

24. The mutant according to paragraplh 23 wherein the mutant is the S. cerevisiae Cdc24-m1 mutant (SEQ. I.D. No: 4), the S. cerevisiae Cdc24-m2 mutant (SEQ. I.D. No: 6) and the S. cerevisiae Cdc24-m3 mutant (SEQ. I.D. No: 8)

25. A method of medical treatent comprising the step of administering a protein sequence shown as SEQ I.D. No: 2 or a derivative, fragment, variant or homologue thereof.

26. A method according to paragraph 25 comprising the step of administering a fragment of the protein sequence shown as SEQ I.D. No: 2, wherein the fragment is the 19 amino acid Cdc24 fragment SEQ. I.D. No: 21.

27. A method according to paragraph 25 comprising the step of administering a homologue of the protein sequence shown as SEQ I.D. No: 2, wherein the homologue is the C.albicans Cdc24 76 amino acid fragment SEQ. I.D. No: 34.

28. A method of medical treatment comprising the step of administering a mutant of the protein sequence shown as SEQ I.D. No: 2 or a derivative, fragment, variant or homologue thereof for use in medicine.

29. A method according to paragraph 28 wherein the mutant is selected from the group comprising S. cerevisiae Cdc24-m1 76 amino acid matant (SEQ. I.D. No: 4), the S. cerevisiae Cdc24-m2 76 amino acid mutant (SEQ. I.D. No. 6) and the S. cerevisiae Cdc24-m3 76 amino acid mutant (SEQ. I.D. No: 8).

30. A method according to paragraph 28 wherein the method comprises the step of administering a fragment of a mutant of the protein sequence shown as SEQ I.D. No: 2, wherein the fragment is selected from the group comprising the S. cerevisiae Cdc24-m1 mutant 19 amino acid fragment (SEQ. I.D. No: 18), the S. cerevisiae Cdc24-m2 mutant 19 amino acid fragment (SEQ. I.D. No: 19) and the S. cerevisiae Cdc24-m3 mutant 19 amino acid fragment (SEQ. I.D. No: 20).

31. A method of modulating the growth behaviour of cells comprising the step of administering a protein sequence shown as SEQ I.D. No: 2 or a derivative, fragment, variant or homologue thereof.

32. A method according to paragraph 31 comprising the step of administering a fragment of the protein sequence shown as SEQ I.D. No: 2, wherein the fragment is the 19 amino acid S. cerevisiae Cdc24 fragment SEQ. I.D. No: 21.

33. A method according, to paragraph 31 compnising the step of admninistering a homologue of the protein sequence shown as SEQ I.D. No: 2, wherein the homologue is the C.albicans Cdc24 76 ino acid fragment SEQ. I.D. No: 34.

34. A method of modulating the growth behaviour of cells comprising the step of administering a mutant of the protein sequence shown as SEQ I.D. No: 2 or a derivative, fragment, variant or homologue thereof for use in medicine.

35. A method according to paragraph 31 wherein the mutant is selected from the group comprising the S. cerevisiae Cdc24-ml 76 amino acid mutant (SEQ. I.D. No: 4), the S. cerevisiae Cdc24-m2 76 amnino acid mutant (SEQ. I.D. No: 6) and the S. cerevisiae Cdc24-m3 76 amino acid mutant (SEQ. I.D. No: 8).

36. A method according to paragraph 31 wherein the method comprises the step of administering a fragment of a mutant of the protein sequence shown as SEQ I.D. No: 2, wherein the fragment is selected from the group comprising the S. cerevisiae Cdc24-m1 mutant 19 amino acid fragment (SEQ. I.D. No: 18), the S. cerevisiae Cdc24-m2 mutant 19 amino acid fragment (SEQ. l.D. No: 19) and the S. cerevisiae Cdc24-m3 mutant 19 amino acid fragment (SEQ. I.D. No, 20).

37. Use of a protein sequence shown as SEQ I.D. No: 2 or a derivative, fragment, variant or homologue thereof in a screen to identify one or more agents that are capable of affecting the interaction of Cdc24p or a homologue thereof with a Gβ or an associated Rho-family GTPase.

38. The use according to paragraph 37 wherein a homologue of the protein sequence shown as SEQ I.D. No: 2 is used and wherein the homologue is the C.albicans Cdc24 76 amino acid fragment SEQ. I.D. No: 34

39. Use of a mutant of a protein sequence shown as SEQ I.D. No: 2 or a derivative, fragment, variant or homologue thereof in a screen to identify one or more agents that are capable of affecting the interaction of Cdc24p or a homologue thereof with a Gβ or an associated Rho-family GTPase.

40. The use according to paragraph 39 wherein the mutant is selected from the group comprising the S. cerevisiae Cdc24-m1 76 amino acid mutant (SEQ. I.D. No: 4), the S. cerevisiae Cdc24-m2 76 amino acid mutant (SEQ I.D. No: 6) and the S.cerevisiae Cdc24-m3 76 amino acid mutant (SEQ. I.D. No: 8).

41. An assay comprising contacting an agent with a protein sequence shown as SEQ I.D. No: 2 or a derivative, fragment, variant or homologue thereof in the presence of a Gβ capable of being associated with Cdc24p or a homologue thereof; and detenmining whether the agent is capable of affecting the interaction of the protein sequence with the Gβ or the Rio-family GTPase.

42. An assay according to paragraph 41 wherein the agent is contacted with a homologue of the protein sequence shown as SEQ. I.D. No: 2, said homologue being the C.albicans Cdc24 76 amino acid fragment SEQ. I.D. No: 34.

43. An assay comprising contacting an agent with a mutant of a protein sequence shown as SEQ I.D. No: 2or a derivative, fragment, variant or homologue thereof in the presence of Gβ capable of being associated with Cdc24p or a homologue thereof; and determining whether the agent is capable of affecting the interaction of the mutant protein sequence with the Gβ or the Rho-family GTPase.

44. An assay according to paragraph 43 wherein the mutant is selected from the group comprising S. cerevisiae Cdc24-m1 76 amino acid mutant (SEQ. I.D. No: 4), the S. cerevisiae Cdc24m2 76 amino acid mutant (SEQ. I.D. No: 6) and the S. cerevisiae Cdc24m3 76 amino acid mutant (SEQ I.D. No: 8).

45. An assay according to paragraph 43 wherein the assay comprises contacting an agent with a fragment of a mutant of the protein sequence shown as SEQ I.D. No: 2 and wherein the fragment is selected from the group comprising the S. cerevisiae Cdc24-m1 mutant 19 amino acid fragment (SEQ. I.D. No: 18), the S. cerevisiae Cdc24-m2 mutant 19 amino acid fragment (SEQ. I.D. No: 19) and the S. cerevisiae Cdc24-m3 mutant 19 amino acid fragment (SEQ I.D. No: 20).

46. A kit comprising a protein sequence shown as SEQ I.D. No: 2 or a derivative, fragment, variant or homologue thereof; and a Gβ capable of being associated with Cdc24p or a homologue thereof.

47. A kit according to paragraph 46 wherein the kit comprises a homologue of the protein sequence shown as SEQ. I.D. No: 2, said homologue being the C.albicans Cdc24 76 amino acid fragment SEQ. I.D. No: 34.

48. A kit comprising a mutant of a protein sequence shown as SEQ I.D. No: 2 or a derivative, fragment, variant or homologue thereof, and a Gβ capable of being associated with Cdc24p or a homologue thereof.

49. A kit according to paragraph 48 wherein the mutant is selected from the group comprising S. cerevisiae Cdc24-m1 76 amino acid mutant (SEQ. I.D. No: 4), the S. cerevisiae Cdc24-m2 76 amino acid mutant (SEQ. I.D. No: 6) and the S.cerevisiae Cdc24-m3 76 amino acid mutant (SEQ. I.D. No: 8).

50. A kit according to paragraph 48 wherein the kit comprises a fragment of a mutant of the protein sequence shown as SEQ I.D. No: 2 and wherein the fragment is selected from the group comprising the S. cerevisiae Cdc24-m1 mutant 19 amino acid fragment (SEQ. I.D. No: 18), the S. cerevisiae Cdc24-m2 mutant 19 amino acid fragment (SEQ. I.D. No: 19) and the S. cerevisiae Cdc24-m3 mutant 19 amino acid fragment (SEQ. I.D. No: 20).

51. A GEF capable of interacting with a Gβ such that the interaction provides a connection between G protein coupled receptor activation and polarised cell growth.

52. An agent capable of affecting a GEF/Gβ interaction, which interaction provides a connection between G protein coupled receptor activation and polarised cell growth.

53. An assay method comprising the use of the sequence presented in SEQ ID No 4 or a nucleotide sequence coding for same.

54. Use of an agent identified by the assay of any one of claims 14, 16, 41, 43 in a method of modulating cell growth.

55. A method of medical treatment according to claim 5, wherein the method is for treatment of fungal infection.

56. A method of medical treatment according to claim 6, wherein the method is for treatment of fungal infection.

57. A mnethod of medical treatment according to claim 7, wherein the method is for treatment of fungal infection.

58. A method of medical treatment according to claim 25, wherein the method is for treatment of fungal infection.

59. A method of medical treatment according to claim 26, wherein the method is for treatment of fungal infection.

60. A method of medical treatment according to claim 27, wherein the method is for treatment of fungal infection.

61. A method of medical treatment according to claim 28, wherein the method is for treatment of fungal infection.

62. A method of medical treatment according to claim 29, wherein the method is for treatment of fungal infection.

63. A method of medical treatment according to claim 30, wherein the method is for treatment of fungal infection.

64. A mutant of a STE4 nucleotide sequence (SEQ I.D. No: 10) or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

65. The mutant, derivative, fraginent, variant or homologue thereof according to claim 64, wherein the mutant is SEQ. I.D. No: 12 or SEQ. I.D. No: 14.

By way of example, in a broad aspect, the present invention provides a nucleotide sequence shown as SEQ. I.D. No: 1 or a derivative, fragment, variant or homologue thereof, wherein the expression product of the nucleotide sequence has the capability of not substantially affecting the interaction of Gβ with GEF or a homologue thereof that is usually capable of being associated therewith.

As mentioned above, the identification in S. cerevisiae of hitherto unrecognised regions that play a key role in the interaction of cellular components has broad implications—not only for the design of anti-fungal drugs, such as those that could be directed against the yeast Candida, but also in the screening and design of agents that can affect oncogenes such as Dbl, in particular proto-Dbl.

However, a complexity of working with Candida species, such as C. albicans, is that the organism is diploid and in a number of cases, the two alleles in the diploid organism have diverged resulting in alleles with different and/or non-identical function. By way of example, an academic consortium accessible at http://alccs.med.umn.edu/Candida.html have annotated, from Blast similarity searches, some small portions of the C. albicans gene (CDC24) which encodes the Cdc24 protein (Cdc24p) Using a shotgun procedure, this academic consortium has only identified small portions of the CDC24 gene encoding Cdc24p and these portions have only been annotated as CDC24 because they pick up the S. cerevisiae CDC24 in a BLAST search. However, the intact Candida gene encoding CDC24 has not bcen annotated as a considerable number of the regions of the C. albicans CDC24 do not line up well with S. cerevisiae CDC24.

Thus, in one aspect, the present invention seeks to overcome the problems associated with the cloning and characterisation of the CDC24 gene obtainable from C. albicans.

Thus, according to one broad aspect of the present invention there is provided a GDP-GTP Exchange Factor (GEF) obtainable from C. albicans wherein the GEF is Cdc24p and wherein the Cdc24p GEF is capable of interacting with proteins such as Gβ. As shown below, these interactions are necessawy for polarised cell growth and hence are appropriate anti-fungal targets.

These and other aspects of the present invention are set out in the following numbered paragraphs.

66. A nucleotide sequence shown as SEQ I.D. No: 23 or a derivative, fragment, variant or homologue thereof, wherein the expression product of the nucleotide sequence has the capability of not substantially affecting the interaction of Gβ with Cdc24p or a homologue thereof that is usually capable of being associated therewith.

67. A fragment of the nucleotide sequence according to paragraph 66 wherein the fragment is the C. albicans 76 amino acid fragment SEQ. I.D. No: 34 or the C. albicans 19 amino acid fragment SEQ. I.D. No: 35

68. A mutant of the nucleodde sequence shown as SEQ I.D. No: 23 or a derivative, fragment, variant or homologne thereof, wherein the expression product of the mutant nucleotide sequence has the capability of substantially affecting the interaction of Gβ with Cdc24p or a homologue thereof that is usually capable of being associated therewith.

69. A method of medical treatment comprising the step of administering a nucleotide sequence shown as SEQ I.D. No: 23 or a derivative, fragment, variant or homologue thereof.

70. A method of medical treatment according to paragraph 69 wherein the fragment comprises nucleotide residues 508 to 735 of the C. albicans Cdc24 gene presented as SEQ. I.D No: 23.

71. A method of medical treatment comprising the step of administering a mutant of the nucleotide sequence shown as SEQ I.D. No: 23 or a derivative, fragment, variant or homologue thereof or the expression product thereof.

72. A method of affecting the growth behaviour of cells comprising the step of administering the nucleotide sequence shown as SEQ I.D. No: 23 or a derivative, fragment, variant or honologue thereof or the expression product thereof to the cells.

73 A method of affecting the growth behaviour of cells according to paragraph 72, wherein the fragment comprises nucleotide residues 508 to 735 of the C. albicans Cdc24 gene presented as SEQ. I.D. No: 23.

74. A method of affecting the growth behaviour of cells comprising the step of administering a mutant of the nucleotide sequence shown as SEQ I.D. No: 23 or a derivative, fragment, variant or homologue thereof or the expression product thereof to the cells.

75. Use of a nucleotide sequence shown as SEQ I.D. No: 23 or a derivative, fragment, variant or homologue thereof or the expression product thereof in a screen to identify one or more agents that are capable of affecting the interaction of Cdc24p or a homologue thereof with a Gβ or an associated Rho-family GTPase.

76. The use according to paragraph 75, wherein the fragment comprises nucleotide residues 508 to 735 of the C. albicans Cdc24 gene presented as SEQ. I.D. No: 23.

77. Use of a mutant of a nucleotide sequence shown as SEQ I.D. No: 23 or a derivative, fragment, variant or homologue thereof or the expression product thereof in a screen to identfy one or more agents that are capable of affectng the interaction of Cdc24p or a homologue thereof with a Gβ or an associated Rho-family GTPase.

78. An assay comprising contacting an agent wilh a nucleotide sequence shown as SEQ I.D. No: 23 or a derivative, fragment, variant or homologue thereof or the expression product thereof in the presence of a Gβ capable of being associated with Cdc24p or a homologue thereof; and determniung whether the agent is capable of affecting the interaction of the nucleotide sequence or the expression product with the Gβ.

79. An assay according to paragraph 78 wherein the fragment comprises nucleotide residues 508 to 735 of the C. albicans Cdc24 gene presented as SEQ. I.D. No: 23.

80. An assay comprising contactig an agent with a mutant of a nucleotide sequence shown as SEQ I.D. No: 23 or a derivative, fragnent, variant or homologue thereof or the expression product thereof in the presence of a Gβ capable of being associated with Cdc24p or a homologue thereof; and determining whether the agent is capable of affecting the interaction of the mutant nucleotide sequence or the expression product with the Gβ.

81. A kit comprising a nucleotide sequence shown as SEQ. I.D. No: 23 or a derivative, fragment, variant or homologue thereof or the expression product thereof; and a Gβ capable of being associated with Cdc24p or a homologue thereof.

82. A kit according to paragraph 81 comprising a fagment of SEQ. I.D. No: 23, wherein the fragment comprises nucleotide residues 508 to 735 of the C. albicans Cdc24 gene presented as SEQ. I.D. No: 23.

83. A kit comprising a mutant of a nucleotide sequence shown as SEQ I.D. No: 23 or a derivative, fragment, variant or homologue thereof or the expression product thereof; and a Gβ capable of being associated with Cdc24p or a homologue thereof.

84. A protein sequence shown as SEQ I.D. No: 24 or a derivative, fragment, variant or homologue thereof, wherein the protein has the capability of not substantially affecting the interaction of Gβ with Cdc24p or a homologue thereof that is usually capable of being associated with the Cdc24p or the homologue thereof.

85. A fragment of the protein sequence shown as SEQ. I.D. No: 24 according to paragraph 19 wherein the fragment is SEQ. I.D. No: 34 or SEQ. I.D. No: 35.

86. A mutant of the protein sequence shown as SEQ I.D. No: 24 or a derivative, fragment, variant or homologue thereof, wherein the mutant protein has the capability of substantially affecting the interaction of Gβ with Cdc24p or a homologue thereof that is usually capable of being associated with the Cdc24p or the homologue thereof.

87. A method of medical treatment comprising the step of administering a protein sequence shown as SEQ I.D. No: 2 or a derivative, fragment, variant or homologue thereof.

88. A method according to paragraph 87 comprising the step of administering a fragent of the protein sequence shown as SEQ I.D. No: 2, wherein the fragment is the 19 amino acid Cdc24 fragment SEQ. I.D. No: 35 or the 76 amino acid Cdc24 fragment SEQ. I.D. No: 34.

89. A method of medical treatment comprising the step of administering a mutant of the protein sequence shown as SEQ I.D. No: 24 or a derivative, fragment, variant or homologue thereof for use in medicine.

90. A method of modulating the growth behaviour of cells comprising the step of administering a protein sequence shown as SEQ I.D. No: 24 or a derivative, fragment, variant or homologue thereof.

91. A method according to paragraph 90 comprising the step of administering a fragment of the protein sequence shown as SEQ I.D. No: 2, wherein the fragment is the 19 amino acid Cdc24 fragment SEQ. I.D. No: 35 or the 76 amino acid Cdc24 fragment SEQ. I.D. No: 34.

92. A method of modulating the growth behaviour of cells comprising the step of administering a mutant of the protein sequence shown as SEQ I.D. No: 24 or a derivative, fragment, variant or honiologue thereof for use in medicine.

93. Use of a protein sequence shown as SEQ I.D. No: 24 or a derivative, fragment, variant or homologue thereof in a screen to identify one or more agents that are capable of affecting the interaction of Cdc24p or a homologue thereof with a Gβ or an associated Rho-family GTPase.

94. The use according to paragraph 93 wherein a fragment of the protein sequence shown as SEQ I.D. No: 2 is used and wherein the fragment is the 19 amino acid Cdc24 fragment SEQ. I.D. No: 35 or the 76 amino acid Cdc24 fragment SEQ. I.D. No: 34.

95. Use of a mutant of a protein sequence shown as SEQ I.D. No: 24 or a derivative, fragment, variant or hoinologue thereof in a screen to identify one or more agents that are capable of affecting the interaction of Cdc24p or a homologue thereof with a Gβ or an associated Rho-family GTPase.

96. An assay comprising contacting an agent with a protein sequence shown as SEQ I.D. No: 24 or a derivative, fragment, variant or homologue thereof in the presence of a Gβ capable of being associated with Cdc24p or a homologue thereof; and determining whether the agent is capable of affecting the interaction of the protein sequence with the Gβ or the Rho-family GTPase.

97. An assay according to paragraph 96 wherein the agent is contacted with a fragment of the protein sequence shown as SEQ. ID. No: 2, wherein said fragment is the 19 amino acid Cdc24 fragment SEQ. I.D. No 35 or the 76 amino acid Cdc24 fragment SEQ. I.D. No. 34.

98. An assay comprising contacting an agent with a mutant of a protein sequence shown as SEQ I.D. No: 24 or a derivative, fragment, variant or homologue thereof in the presence of Gβ capable of being associated with Cdc24p or a homologue thereof; and determiining whether the agent is capable of affecting the interaction of the mutant protein sequence with the Gβ or the Rho-family GTPase.

99. A kit conmprising a protein sequence shown as SEQ I.D. No: 24 or a derivative, fragment, vanant or homologue thereof; and a Gβ capable of being associated with Cdc24p or a homologue thereof.

100. A kit aceording to paagraph 99 wherein the kit comprises a fragment of the protein sequence shown as SEQ. I.D. No: 2, wherein said fragment is the 19 amino acid Cdc24 fragment SEQ. I.D. No: 35 or the 76 amino acid Cdc24 fagment SEQ. I.D. No: 34.

101. A kit compnrsing a mutant of a protein sequence shown as SEQ I.D. No: 24 or a derivative, fragment, variant or homologue thereof, and a Gβ capable of being associated with Cdc24p or a homologue thereof.

102. An assay method comprising the use of the sequence presented in SEQ I.D. No 34 or a nucleotide sequence coding for same

103. Use of an agent identified by the assay of any one of paragraphs 78, 79, 80, 96, 97, 98 or 102 in a method of modulating cell growth.

104. A method of medical treatnent according to any one of paragraphs 87, 88 or 89 wherein the method is a method of treatment of fungal infection.

105. A method according to paragraph 104 wherein the fuingal infection is Candida albicans infection.

106. A mutant of a STE4 nucleotide sequence (SEQ I.D. No: 10) or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

107. The mutant, derivative, fragment, variant or homologue thereof according to paragraph 106, wherein the mutant is SEQ. I.D. No: 12 or SEQ. I.D. No: 14.

108. Use of mutant, derivative, fragment, variant or homologue thereof according to paragraph 106 or paragraph 107, in a screen to identify one or more agents that are capable of affecting the non-interactive behaviour of the Ste4p mutant encoded by said mutant, derivative, fragment, variant or homologue thereof with Cdc24p or a homologue thereof.

109. An assay comprising contacting an agent with a mutant of a STE4 nucleotide sequence (SEQ I.D. No: 10) or a derivative, fragment, variant or homologue thereof or the expression product thereof in the presence of Cdc24p or a homologue thereof; and determining wheter the agent is capable of affecting the non-interactive behaviour of the STE4 mutait nucleotide sequence or the expression product with the Cdc24p.

110. The assay according to paragraph 109 wherein the mutant is SEQ. I.D. No: 12 or SEQ. I.D. No: 14.

111. A kit comprising a mutant of a nucleotide sequence shown as SEQ I.D. No: 10 or a derivative, fragment, variant or homologue thereof or the expression product thereof; and a Cdc24p capable of being associated with Ste4p or a homologue thereof.

112. A mutant of the Ste4p protein sequence shown as SEQ I.D. No: 11 or a derivative, fragment, variant or homologue thereof, wherein the mutant protein has the capability of substantially affecting the interaction of Gβ with Cdc24p or a homologue thereof that is usually capable of being associated the Cdc24p or the homologue thereof.

113. The mutant, derivative, fragment, variant or homologue thereof according to paragraph 112, wherein the mutant is SEQ. I.D. No: 13 or SEQ. I.D. No 15.

114. Use of the mutant, derivative, fragment, variant or homnologue thereof according to paragraph 112 or 113, in a screen to identify one or more agents that are capable of affecting the non-interactive behaviour of the Ste4p mutant, derivative, fragment, variant or homologue thereof with Cdc24p or a homologue thereof.

115. An assay comprising contacting an agent with a mutant of a Ste4p amino acid sequence (SEQ I.D. No: 11) or a derivative, fragment, variant or homologue thereof in the presence of Cdc24p or a homologue thereof; and determining whether the agent is capable of affecting the non-interactive behaviour of the Ste4p mutant with Cdc24p.

116. The assay according to paragraph 115 wherein the mutant is SEQ. I.D. No: 13 or SEQ. I.D. No: 15.

117. An assay comprising contacting a mutant of a Ste4p protein sequence (SEQ I.D. No: 11) or a derivative, fragment, variant or homologue thereof, wherein the mutant protein has the capability of substantially affecting the interaction of Gβ with Cdc24p, with a Cdc24p homologue; and determining whether the Cdc24p homologue is capable of affecting the non-interactive behaviour of the Ste4p mutant with Cdc24p.

118. An assay according to paragraph 117 wherein the Cdc24p homologue is a homologue of Cdc24p selected from Cdc24-m1 (SEQ. I.D. No: 4), Cdc24-m2 (SEQ. I.D. No: 6) Cdc24-m3 (SEQ. I.D. No: 8).

119. A kit comprising a mutant of the Ste4p protein sequence shown as SEQ I.D. No: 11 or a derivative, fragment, variant or homologue thereof or the expression product thereof; and a Cdc24p capable of being associated with Ste4p or a homologue thereof.

120. An assay comprising contacting a mutant of a Ste4p protein sequence (SEQ I.D. No: 11) or a derivative, fragment, variant or homologue thereof, wherein the mutant protein has the capability of substantially affecting the interaction of Gβ with Cdc24p, with a Cdc24p homologue; and determining whether the Cdc24p homologue is capable of affecting the non-interactive behaviour of the Ste4p mutant with Cdc24p.

121. An assay according to paragraph 120 wherein the Cdc24p homologue is a homnologue of Cdc24p selected from Cdc24-m1 (SEQ. I.D. No: 4), Cdc24-m2 (SEQ. I.D. No: 6) Cdc24-m3 (SEQ. I.D. No: 8).

122. A kit comprising a mutant of the Ste4p protein sequence shown as SEQ I.D. No: 11 or a derivative, fragment, variant or homologue thereof or the expression product thereof; and a Cdc24p capable of being associated with Ste4p or a homologue thereof.

123 A mutant of a Cdc42 nucleotide sequence or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

124. Use of mutant, derivative, fragment, variant or homologue thereof according to paragraph 120, in a screen to identify one or more agents that are capable of affecting the interaction of the CDC42p encoded by said mutant, derivative, fragment, variant or homologue thereof with Cdc24p or a homologue thereof.

125. An assay compnsing contacting an agent with a mutant of a Cdc42 nucleotide sequence or a derivative, fragment, variant or homologue thereof or the expression product thereof in the presence of Cdc24p or a homologue thereof; and determining whether the agent is capable of affecting the interaction of the Cdc42 nucleotide sequence or the expression product with the Cdc24p.

126. A kit comprising a mutant of a CDC42 nucleotide sequence or a derivative, fragment, variant or homologue thereof or the expression product thereof; and a Cdc24p capable of being associated with Cdc42p or a homologue thereof.

127. A mutant of a Cdc42p protein, wherein the mutant protein has the capability of substantially affecting the interaction of Gβ with Cdc24p or a homologue thereof that is usually capable of being associated with he Cdc24p or the homologue thereof.

128. Use of the mutant according to paragraph 127, in a screen to identify one or more agents that are capable of affecting the interactive behaviour of the Cdc42p mutant, with Cdc24p or a homologue thereof.

By way of example, in a broad aspect, the present invention provides a nucleotide sequence shown as SEQ. I.D. No: 1 or SEQ. I.D No: 23, or a derivative, fragment, variant or homologue thereof, wherein the expression product of the nucleotide sequence has the capability of not substantially affecting the interaction of proteins such as Gβ with a GEF or a homologue thereof that is usually capable of being associated therewith.

As used herein, the term “Gβ” includes Gβ and any Gβ associated protein such as Ste4p/Ste18p and/or a Rho-family GTPase (such as Cdc42p).

The term “expression product of the nucleotide sequence has the capability of not substantially affecting the interaction of proteins such as Gβ with GEF or a homologue thereof that is usually capable of being associated therewith” means that if the expression product were to be present within the GEF and the GEF were to be contacted with a protein such as Gβ then the expression product would not substantially affect the interaction of a protein such as Gβ with the GEF.

Thus, alternatively expressed, the present invention covers a nucleotide sequence shown as SEQ. I.D. No: 1 or SEQ. I.D. No: 23, or a derivative, fragment, variant or homologue thereof, wherein the expression product of the nucleotide sequence has the capability of not substantially affecting the interaction of a protein such as Gβ with a GEF or a homologue thereof that is usually capable of being associated therewith if the expression product were to be present within the GEF and the GEF were to be contacted with proteins such as Gβ.

With this aspect of the present invention, the expression product need not necessarily be present within the GEF and/or the GEF need not necessarily be contacted with a protein such as Gβ. By way of example, the expression product can be part of a truncated GEF and/or part of a fused protein. However, if the expression product were present within GEF, then preferably the GEF is not in its natural environment. By way of example, the GEF can be in an isolated form—such as in an assay device. Likewise, if the expression product were contacted with a protein such as Gβ then preferably the protein such as Gβ is not in its natural environment. By way of example, the protein such as Gβ can be in an isolated form—such as in an assay device.

The present invention also covers a mutant of the nucleotide sequence shown as SEQ. I.D. No: 1 or SEQ. I.D. No: 23, or a derivative, fragment, variant or homologue thereof, wherein the expression product of the mutant nucleotide sequence has the capability of substantially affecting the interaction of a protein such as Gβ with a GEF or a homologue thereof that is usually capable of being associated therewith.

The term “expression product of the mutant nucleotide sequence has the capability of substantially affecting the interaction of a proein such as Gβ with a GEF or a homologue thereof that is usually capable of being associated therewith” means that if the expression product were to be present within a GEF like entity (such as GEF bearing that mutation) and that GEF like entity were to be contacted with a protein such as Gβ then the expression product would substantially affect the interaction of Gβ with that GEF like entity.

Thus, alternatively expressed, the present invention also covers a mutant of the nucleotide sequence shown as SEQ. I.D. No: 1 or SEQ. I.D. No: 23 or a derivative, fragment, variant or homologue thereof, wherein the expression product of the mutant nucleotide sequence has the capability of substantially affecting the interaction of a protein such as Gβ with a GEF or a homologue thereof that is usually capable of being associated therewith if the expression product were to be present within GEF and the GEF were to be contacted with a protein such as Gβ.

With this aspect of the present invention, the expression product need not necessarily be present within the GEF like entity and/or the GEF like entity need not necessarily be contacted with the protein such as Gβ. By way of example, the expression product can be part of a truncated GEF and/or part of a fused protein. The GEF like entity may be in an isolated form—such as in an assay device. Likewise, if the expression product were contacted with a protein such as Gβ then preferably the protein such as Gβ is not in its natural environment. By way of example, the protein such as Gβ can be in an isolated form—such as in an assay device.

In one preferred aspect, the GEF is Cdc24p. Other suitable GEFs have been mentioned above.

Thus, the present invention also covers in a broad aspect a nucleotide sequence shown as SEQ. I.D. No: 1 or SEQ. I.D. No: 23, or a derivative, fragment, variant or homologue thereof, wherein the expression product of the nucleotide sequence has the capability of not substantially affecting the interaction of Gβ with Cdc24p or a homologue thereof that is usually capable of being associated therewith.

The term “expression product of the nucleotide sequence has the capability of not substantially affecting the interaction of Gβ with Cdc24p or a homologue thereof that is usually capable of being associated therewth” means that if the expression product were to be present within Cdc24p and the Cdc24p were to be contacted with Gβ then the expression product would not substantially affect the interaction of Gβ with Cdc24p.

Thus, alternatively expressed, the present invention covers in a broad aspect a nucleotide sequence shown as SEQ. I.D. No: 1 or SEQ. I.D. No: 23, or a derivative, fragment, variant or homologue thereof, wherein the expression product of the nucleotide sequence has the capability of not substantially affecting the interaction of Gβ with Cdc24p or a homologue thereof that is usually capable of being associated therewith if the expression product were to be present within Cdc24p and the Cdc24p were to be contacted with Gβ.

With this aspect of the present invention, the expression product need not necessarily be present within Cdc24p and/or the Cdc24p need not necessarly be contacted with Gβ. By way of example, the expression product can be part of a trunated Cdc24p and/or part of a fused protein. However, if the expression product is present within Cdc24p, then preferably the Cdc24p is not in its natural environment. By way of examnple, the Cdc24p can be in an isolated form—such as in an assay device. Likewise, if the expression product were contacted with Gβ then preferably the Gβ is not in its natural environment. By way of examnple, the Gβ can be in an isolated form such as in an assay device.

By way of further example, the present invention also covers a mutant of the nucleotide sequence shown as SEQ. I.D. No: 1 or SEQ. I.D. No:.23, or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

The term “expression product of the mutant nucleotide sequence has the capability of substantially affecting the interaction of Gβ with Cdc24p or a homologue thereof that is usually capable of being associated therewith” means that if the expression product were to be present within a Cdc24p like entity (such as Cdc24p bearing that mutation) and that Cdc24p like entity were to be contacted with Gβ then the expression product would substantially affect the interaction of Gβ with that Cdc24p like entity.

With this aspect of the present invention, the expression product need not necessarily be present within the Cdc24p like entity and/or the Cdc24p like entity need not necessarily be contacted with Gβ. By way of example, the expression product can be part of a truncated Cdc24p and/or part of a fused protein. The Cdc24p like entity may be in an isolated form—such as in an assay device. Likewise, if the expression product were contacted with Gβ then preferably the Cβ is not in its natural environment. By way of example, the Gβ can be in an isolated form—such as in an assay device.

In a preferred aspect, the present invention covers the sequences of the present invention in isolated form—in other words the sequences are not in their natural environment and when they have been expressed by their natural coding sequences which are under the control of their natural expression regulatory elements (such as the natural promoter etc.). By way of example the sequences may be in an assay device.

It is to be noted that the nucleotide sequence presented as SEQ. I.D. No: 1 is quite different to the DH domain and the PH domain discussed by Cerione and Zheng (ibid). It is also to be noted that the nucleotide sequence presented as SEQ. I.D. No: 1 is in a region quite different to the DH domain and the PH domain. The nucleotide sequence presented as SEQ ID No: 23 is also quite different to the DH domain and the PH domain discussed by Cerione and Zheng (ibid). Moreover, the nucleotide sequence presented as SEQ ID No: 23 covers regions in addition to the DH domain and the PH domain.

One important asect of the present invention is that we have found it is possible to affect the interaction of Cdc24p with a β subunit (such as Ste4p) or even a βγ subunit (such as Ste4p/Ste18p) of a hetero-trimeric G-protein (hereinafter collectively referred to as “Gβ”). For example the nucleotide sequence (SEQ ID No 1) and its expression product (SEQ ID No 2) may affect the interaction of Cdc24p with a β subunit (such as Ste4p) or even a βγ subunit (such as Ste4p/Ste18p) of a hetero-trimeric G-protein (herein referred to as “Gβ”). Likewise, the nucleotide sequence (SEQ ID No 23) and its expression product (SEQ ID No 24) may affect the interaction of C. albicans Cdc24p with a β subunit (such as Ste4p) or even a βγ subunit (such as Ste4p/Ste18p) of a hetero-trimeric G-protein (herein referred to as “Gβ”). If the interaction is detrimentally affected (such as lost) then this may in turn prevent (or at least reduce) signalling (possibly GEF activity) being passed to the the Rho-famly GTPase (Cdc42p). Hence, the present invention also covers the use of any one or more of the aforementioned aspects of the present invention to have an effect on a signal being passed to the Rho-family GTPases.

The term “derivative, fragment, varant or homologue” in relation to the nucleotide Sequence ID No: 1 of the present invention includes any substitution of, modification of, replacement of, deletion of or addition of one (or more) nucleic acid from or to the sequence providing the resultant nucleotide sequence or the expression product thereof has the capability of not substantially affecting the interaction of Gβ with Cdc24p or a homologue thereof that is usually capable of being associated with the Cdc24p or the homologue thereof. In particular, the term “homologue” covers homology with respect to function. With respect to sequence homology (i.e. similarity), preferably there is at least 75%, more preferably at least 85%, more preferably at least 90% homology to the sequence shown as SEQ. I.D. No: 1 in the attached sequence listings. More preferably there is at least 95%, such as at least 98%, homology to the sequence shown as SEQ. I.D. No: 1 in the attached sequence listings.

The term “derivative, fragment, variant or homologue” in relation to the protein Sequence ID No: 2 of the present invention includes any substitution of, modification of, replacement of, deletion of or addition of one (or more) amino acid from or to the sequence providing the resultant amino acid sequence has the capability of not substantially affecting the interaction of Gβ with Cdc24p or a homologue thereof that is usually capable of being associated with the Cdc24p or the homologue thereof.

In particular, the term “homologue” covers homology with respect to function. With respect to sequence homology (i.e. similarity), preferably there is at least 75%, more preferably at least 85%, more preferably at least 90% homology to thie sequence shown as SEQ. I.D, No: 2 in the attached sequence listings. More preferably there is at least 95%, such as at least 98%, homology to the sequence shown as SEQ. I.D. No: 2 in the attached sequence listings.

An example of a fragment of the expression product of SEQ. I.D. No: 1 that has the capability of not substantially affecting the interaction of Gβ with Cdc24p or a homologue thereof that is usually capable of being associated with the Cdc24p or the homologue thereof is the amino acid sequence presented as SEQ. I.D. No: 21 or SEQ. I.D. No: 22. The present invention also covers nucleotide sequences coding for such sequences.

With respect to the mutated sequences then, in a prefered aspect, the mutated sequence comprises one or more mutations in the region presented as SEQ. I.D. No: 21 or SEQ. I.D. No: 22.

An example of a fragment of the expression product of a mutant SEQ. I.D. No: 1 that has the capability of substantially affecting the interaction of Gβ with Cdc24p or a homologue thereof that is usually capable of being associated with the Cdc24p or the homologue thereof is the amino acid sequence presented as SEQ. I.D. No: 18 or SEQ. I.D. No: 19 or SEQ. I.D. No: 20. The present invention also covers nucleotide sequences coding for such sequences.

As described in the Examples, the 19 amino acid fragment of C. albicans (SEQ. I.D. No: 35) corresponding to the 19 amino acid fagment of the S. cerevisiae Cdc24p with similarity to the human proto-oncogene Dbl shares 89.5% homology with the S. cerevisiae Cdc24p 19 amino acid fragment (SEQ. I.D. No: 21) and the 76 amino acid fragment of C. albicans (SEQ. I.D. No: 34) corresponding to amino acids 170 to 245 in S. cerevisiae shares 75.0% homology with the corresponding S. cerevisiae fragment (SEQ. I.D. No: 2). Such C. albicans fragments are thus further examples of “homologues” of the sequence shown as SEQ. I.D. No: 2.

The term “derivative, fragment, variant or homologue” in relation to the nucleotide Sequence ID No: 23 of the present invention includes any substitution of, modification of, replacement of, deletion of or addition of one (or more) nucleic acid from or to the sequence providing the resultant nucleotide sequence or the expression product thereof has the capability of not substantially affecting the interaction of Gβ with a Cdc24p obtainable from C. albicans or a homologue thereof that is usually capable of being associated with a Cdc24p obtainable from C. albicans or the homologue thereof. In particular, the term “homologue” covers homology with respect to function. With respect to sequence homology (i.e. similarity), preferably there is at least 75%, more preferably at least 85%, more preferably at least 90% homology to the sequence shown as SEQ ID No: 23 in the attached sequence listings. More preferably there is at least 95%, such as at least 98%, homology to the sequence shown as SEQ ID No: 23 in the attached sequence listings.

The term “derivative, fragment, variant or homologue” in relation to the protein Sequence ID No: 24 of the present invention includes any substitution of, modification of, replacement of, deletion of or addition of one (or more) amino acid from or to the sequence providing the resultant amino acid sequence has the capability of not substantially affecting the interaction of Gβ with a Cdc24p obtainable from C. albicans or a homologue thereof that is usually capable of being associated with a Cdc24p obtainable from C. albicans or the homologue thereof. In particular, the term “homologue” covers homology with respect to flinction. With respect to sequence homology (i.e. similarty), preferably there is at least 75%, more preferably at least 85%, more preferably at least 90% homology to the sequence shown as SEQ ID No: 24 in the attached sequence listings. More preferably there is at least 95%, such as at least 98%, homology to the sequence shown as SEQ ID No: 24 in the attached sequence listings.

In particular, the term “homology” as used herein may be equated with the term “identity”. Relative sequence homology (i.e. sequence identity) can be determined by commercially available computer programs that can calculate % homology between two or more sequences. Typical examples of such computer programs are BLAST and CLUSTAL.

Sequence homology (or identity) may moreover be determined using any suitable homology algorithmn using for example default parameters. Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Aligmnent Search Tool) is the heuristic search algorithm employed by the programs blastp, blastn, blastx, tblastn, and tblastx; these programs ascribe significance to their findings using the statistical methods of Karlin and Altschul (see http://www.ncbi.nih.gov/BLAST/blast_help.html) with a few enhancements. The BLAST programs were tailored for sequence similarity searching, for example to ideatify homologues to a query sequence. The programs are not generally useful for motif-style searching. For a discussion of basic issues in similarity searching of sequence databases, see Altschul et al (1994) Nature Genetics 6:119-129.

The five BLAST programs available at http://www.ncbi.nlm.nih.gov perform the following tasks:

blastp compares an amino acid query sequence against a protem sequence database;

blastn compares a nucleotide query sequence against a nucleotide sequence database;

blastx compares the six-frame conceptual translation products of a nucleotide query sequence (both strands) against a protein sequence database;

tblastn compares a protein query sequence against a nucleotide sequence database dynamically translated in all six reading frames (both strands).

tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRlIPTONS Restricts the number of short descriptions of matching sequences reported to the number specified; default limit is 100 descriptions (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a mnatch is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17;191-201, or, for BLASTN, by the DUST program of Tatusov and Lipman (see http//www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Preferably, sequence compaisons are conducted using the simple BLAST search algorithm provided at http;//www.ncbi.nlm.nih.gov/BLAST.

More preferably, sequence comparisons are conducted using the simple BLAST 2 search algorithm provided at http://www.ncbi.nlm.nih.gov/gorf/wblast2.cgi.

Other computer program methods to determine identiy and similarity between the two sequences include but are not limited to the GCG program package (Devereux et al 1984 Nucleic Acids Research 12; 387and FASTA (Atschul et al 1990 J Molec Biol 403-410).

The term “variant” also encompasses sequences that are complementary to sequences that are capable of hybridising to the nucleotide sequences presented herein.

Preferably, the term “variant” encompasses sequences that are complementary to sequences that are capable of hydridising under stringent conditions (eg. 65° C. and 0.1×SSC {1×SSC=0.15 M NaCl, 0.015 Na₃ citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

The term “hybridization” as used herein shall include “the process by which a strand of nucleic acid joins with a coomplementary strand through base pairing” (Coombs I (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Also included within the scope of the present invention are polynucleotide sequences that are capable of hybridizing to a nucleotide sequence of the present invention or other nucleotide sequences coding for a protein sequence of the present invention under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect simnilar or related polynucleotide sequences.

In a preferred aspect, the present invention covers nucleotide sequences that can hybridise to the nucleotide sequence of the present invention under stringent conditions (e.g. 65° C. and 0.1×SSC).

Examples of homologies of Cdc24p include but are not limited to any one or more of the homologues listed above or below, such as proto-Dbl, Bcr, Sos, Vav, ect-2, Ost, Tim, Lbc, Lfc and Dbc.

The term “mutant” in relation to the nucleotide sequence of SEQ. I.D. No: 1 means a variant of SEQ. I.D. No: 1 but wherein that variant or the expression product thereof has the capability of substantially affecting the interaction of Gβ with Cdc24p or a homologue thereof that is usually capable of being associated with the Cdc24p or the homologue thereof.

Preferred mutants of the nucleotide sequence of SEQ. I.D No: 1 include any one or more of the nucleotide sequences presented as SEQ. I.D. No: 3, SEQ. I.D. No: 5 or SEQ. I.D. No: 7.

The term “mutant” in relation to the protein sequence of SEQ. I.D. No: 2 means a variant of SEQ. I.D. No: 2 but wherein that variant has the capability of substantially affecting the interaction of Gβ with Cdc24p or a homologue thereof that is usually capable of being associated with the Cdc24p or the homologue thereof.

Preferred mutants of the protein sequence of SEQ I.D. No: 2 include any one or more of the protein sequences presented as SEQ. I.D. No: 4, SEQ. I.D. No: 6 or SEQ. I.D. No: 8.

The term “mutant” in relation to the nucleotide sequence of SEQ. I.D. No: 23 means a variant of SEQ ID No: 23 but wherein that variant or the expression product thereof has the capability of substantially affecting the interaction of Gβ with a Cdc24p obtainable from C. albicans or a homologue thereof that is usually capable of being associated with the Cdc24p obtainable from C. albicans or the homologue thereof.

The term “mutant” in relation to the protein sequence of SEQ. I.D. No:24 means a variant of SEQ ID No: 24 but wherein that variant has the capability of substantially affecting the interaction of Gβ with a Cdc24p obtainable from C. albicans or a homologue thereof that is usually capable of being associated with the Cdc24p obtainable from C. albicans or the homologue thereof.

The term “growth behaviour” includes growth per se (but not vegetative growth of yeast), growth control and growth orientation of cells. In some aspects, it includes at least growth orientation of cells. The term may also include the mating pattern (e.g. mating per se or mating behaviour) of cells.

For a preferred aspect of the present invention, any one or more of the nucleotide sequences of the present invention or the expression product thereof, or the mutant nucleotide sequences of the present invention or the expression product thereof, or the proteins of the present invention, or the mutant proteins of the present invention may be within a transgenic organism or cell (such as being an integral part thereof)—that is an organism or cell that is not a naturally occurring organism or cell and wherein the organism or cell has been prepared by use of recombinant DNA techniques. The transgenic cell may be part of or contained within tissue.

Preferably, the transgenic organism or cell is a yeast, an animal (such as a mammal) or an animal cell (such as a manumalian cell).

In preferred embodiments, the transgenic organism is a transgenic yeast or a trausgenic mouse.

Transgenic yeast may be prepared by appropriately adapting the teachings of Ito et al Journal of Bacteriology 153 163-168; Rose et al 1991 Methods in yeast genetics: a laboratory course manual Cold Spring Harbor, N.Y.: Cold Spring Harbor Press).

Transgenic mammals or mammalian cells may be prepared by appropriately adapting the teachings of Ausubel et al 1992 Short Protocols in Molecular Biology 2nd Ed. New York: John Wiley and Sons).

The transgenic organism or transgenic cell of the present invention therefore provides a simple assay system that can be used to determine whether one or more agents (e.g. compounds or compositions) have one or more beneficial properties. By way of example, the assay system of the present invention may utilise a mating phenotype and/or the assay system may be a two-hybrid interaction assay.

By way of example, if the transgenic organism is a transgenic yeast which comprises the nucleotide sequence presented as SEQ. I.D. No: 1 or the expression product thereof (namely the protein sequence presented as SEQ. I.D. No: 2) then the yeast could be used to screen for agents that bind to this nucleotide sequence or the expression product thereof and in doing so affect the growth behaviour of the yeast. If an agent produces such a detrimental effect (such as drastically reducing the ability of the yeast to mate), then that agent may also affect the interaction of Gβ with Cdc24p or another Cdc24p entity that is usually capable of being associated therewith. This aspect of the present invention could allow workers to screen for anti-fungal agents, such as agents that could be used to treat or combat Candida.

By way of further example, if the transgenic organism is a transgenic yeast which comprises the nucleotide sequence presented as SEQ. I.D. No: 1 or the expression product thereof then the yeast could be used to screen for agents that bind to this nucleotide sequence or expression product thereof and in doing so affect the growth behaviour of the yeast. If an agent produces a detrimental affect (such as drastically reducing the ability of the yeast to mate), then that agent is likely to detrimentally affect the interaction of Gβ with a homologue of Cdc24p with which it is usually capable of being associated. This could allow workers to screen for compounds or compositions that could for example influence the in vivo expression or behaviour of effect of proto-oncogenes and the like—such as proto-Dbl.

By way of yet another example, if the transgenic organism is a transgenic yeast which comprises a mutant of the nucleotide sequence in accordance with the present invention then the yeast could be used to screen for agents that affect the growth behaviour of the yeast. If an agent produces a marked affect—such as restoration to a normal growth behaviour or a firer detrimental growth behaviour—then workers could screen for compounds or compositions that could for example influence the in vivo expression or behaviour or effect or activity of a Cdc24 homologue, such as, but not limited to proto-oncogenes such as Dbl and/or Vav.

By way of further example, if the transgenic organism is a transgenic yeast which comprises a homologue (e.g. Dbl) of the nucleotide sequence shown as SEQ. I.D. No: 1 or an expression product thereof then workers could see if that homologue or the expression product thereof had an effect on the growth behaviour of yeast, and thus also to see if it had an effect on the interaction of Gβ with a homologue of Cdc24p. In addition, workers could use those transgenic yeast to screen for agents that modified the effect—such as enhance the growth behaviour or detrirnentally affect the growth behaviour. In this aspect, agents that affect the growth behaviour may also influence the activity of oncogenes (or even parts thereof) and therefore have potential as therapeutic agents.

The assays of the present invention may also be used to screen for agents that affect the interaction of Cdc24p or a Cdc24p homologue with Gβ to detennine whether that effect has a downstream effect on a Rho-fanlily GTPase.

For example, with the present invention—such as by use of the assays of the present invention—it is possible to devise and/or to screen for peptide inhibitors which block GEF/Gβ interaction. In this regard, peptides and peptidyl derivatives based regions encompassing mutants may be used to block and/or antagonise GEF (such as the proto-oncogenes Dbl or Vav) Gβ interaction. Derivatives of these peptides (including peptide mimics) which bind with higher affimity may also be used. The perturbation of these interactions may be of therapeutic value for example in treatment of cancers.

In addition, by use of the present invention it is possible to devise simple yeast based assay systems (utilising mating function and interaction reporters). These assay systems will be extremely useful for high through-put screening to identify molecules perturbing the GEF/Gβ interaction.

In addition, it is possible to devise and/or screen for agents that can modulate (e.g. interact), preferably selectively modulate (interact), with and affect Cdc24p/Gβ interactions. Hence, it would be possible to devise and/or to screen for anti-fungal agents directed at invasive and/or pathogenic yeasts such as, but not limited to Candida albicans and/or Cryptococcus neoformans.

By way of example, if the transgenic organism is a transgenic yeast which comprises the nucleotide sequence presented as SEQ ID No: 23 or the expression product thereof (namely the protein sequence presented as SEQ ID No: 24) then the yeast could be used to screen for agents that bind to this nucleotide sequence or the expression product thereof and in doing so affect the growth behaviour of the yeast. If an agent produces such a detrimental effect (such as drastically reducing the ability of the yeast to mate), then that agent may also affect the interaction of Gβ with a Cdc24p obtainable from C. albicans or another Cdc24p entity that is usually capable of being associated therewith. This aspect of the present invention could allow workers to screen for anti-fungal agents, such as agents that could be used to treat or combat Candida.

By way of further example, if the transgenic organism is a transgenic yeast which comprises the nucleotide sequence presented as SEQ ID No: 23 or the expression product thereof then the yeast could be used to screen for agents that bind to this nucleotide sequence or expression product thereof and in doing so affect the growth behaviour of the yeast. If an agent produces a detrimental affect (such as drastically reducing the ability of the yeast to mate), then that agent is likely to detrimentally affect the interaction of Gβ with a homologue of C. albicans Cdc24p with which it is usually capable of being associated.

By way of further exarnple, if the transgenic organism is a transgenic yeast which comprises a mutant of the nucleotide sequence in accordance with the present invention then the yeast could be used to screen for agents that affect the growth behaviour of the yeast.

By way of fiurther example, if the transgenic organism is a transgenic yeast which comprises a homologue of the nucleotide sequence shown as SEQ ID No: 23 or an expression product thereof then workers could see if that homologue or the expression product thereof had an effect on the growth behaviour of yeast, and thus also to see if it had an effect on the interaction of Gβ with a homologne of the Cdc24p obtainable from C. albicans. In addition, workers could use those transgenic yeast to screen for agents that modified the effect—such as enhance the growth behaviour or detrimentally affect the growth behaviour. In this aspect, agents that affect the growth behaviour could have potential as anti-fuingal agents.

The assays of the present invention may also be used to screen for agents that affect the interaction of a Cdc24p obtainable from C. albicans or a homologue of a Cdc24p obtainable from C. albicans with Gβ to determine whether that effect has a downstream effect on a Rho-family GTPase.

For example, with the present invention—such as by use of the assays of the present invention—it is possible to devise and/or to screen for peptide inhibitors which block GEF/Gβ interaction. In this regard peptides and peptidyl derivatives based regions encompassing mutants may be used to block and/or antagonise a GEF, for example obtainable from C. albicans Gβ interaction. Derivatives of these peptides (including peptide mimics) which bind with higher affinity may also be used. The perturbation of these interactions may be of therapeutic value, for example in treatment of fungal disorders.

In addition, by use of the present invention it is possible to devise simple yeast based assay systems (utilising mating function and interaction reporters). These assay systems will be extremely useful for high through-put screening to identify molecules perturbing a GEF/Gβ interaction wherein the GEF is obtainable fromn C. albicans or is a homologue thereof.

In addition, it is possible to devise and/or screen for agents that can modulate (e.g. interact), preferably selectively modulate (interact), with and affect Cdc24p/Gβ interactions wherein the Cdc24p is obtainable from C. albicans or is a homologue thereof. Hence, it would be possible to devise and/or to screen for anti-fungal agents directed at invasive and/or pathogenic yeasts such as, but not limited to Candida albicans and/or Cryptococcus neoformans and/or Aspergillus species such as Aspergillus niger.

If the assay of the present invention utilises a tansgenic organism according to the present invention then tramsgenic organism may comprise nucleotide sequences etc, that are additional to the nucleotide sequences of the present invention in order to maintain the viability of the transgenic organsm.

In the assays of the present invention, the agent can be any suitable compound, composition as well as being (or even including) a nucleotide sequence of interest or the expression product thereof. Hence, if any one of the nucleotide sequences of the present invention are contained withinin a transgenic organism—such as a transgenic yeast—then that transgenic organism may also contain that nucleotide sequence of interest. If the agent is a nucleotide sequence, then the agent may be, for example, nucleotide sequences from organisms (e.g. higher organisms—such as eukaryotes) that restore or increase the growth behaviour. Agents which affect the growth beliaviour may also influence the activity of homologous oncogenes and may therefore be potential therapeutic agents.

The following samples were deposited in accordance with the Budapest Treaty at the recognised depositary of The National Collections of Industal and Marine Bacteria Limited (NCIMB) at 23 St. Machar Drive, Aberdeen, Scotland, United Kingdom, AB2 1RY on Oct. 3, 1997:

E. coli CMK603 PRS414CDC24 (WT)—Deposit Number NCIMB 40898

E. coli CMK603 PRS414CDC24 (M1)—Deposit Number NCIMB 40899

E. coli CMK603 PRS414CDC24 (M2)—Deposit Number NCIMB 40900

E. coli CMK603 PRS414CDC24 (M3)—Deposit Number NCIMB 40901

Deposit NCIMB 40898 is in respect of cdc24 (wt); Deposit NCIMB 40899 is in respect of cdc24-m1; Deposit NCIMB 40900 is in respect of cdc24-m2; Deposit NCIMB 40901 is in respect of cdc24-m3.

In accordance with a preferred aspect of the present invention, a nucleotide sequence is obtainble from, or the protein is evressable from the nucleotide sequence contained within, the respective deposit. By way of example, the respective nucleotide sequence may be isolated from the respective deposit by use of appropriate restriction enzymes or by use of PCR techniques.

### BRIEF DESCRIPTION OF THE DRAWINGS

The present invention will now be described only by way of example, in which reference is made to the following Figures:

FIG. 1 which presents some photographs and a graph;

FIG. 2 which presents some images and graphs;

FIG. 3 which presents some photographs, a sequence, and a pictorial representation of Cdc24 and DBD Cdc24; and

FIG. 4 which presents a pictorial representation of a cellular interaction.

FIG. 5 which presents the nucleotide sequence (SEQ ID No 23) and the translated protein sequence (SEQ ID No 24) obtainable from C. albicans;

FIG. 6 which presents a BLAST line up of S. cerevisiae Cdc24p and C. aibicans Cdc24p.

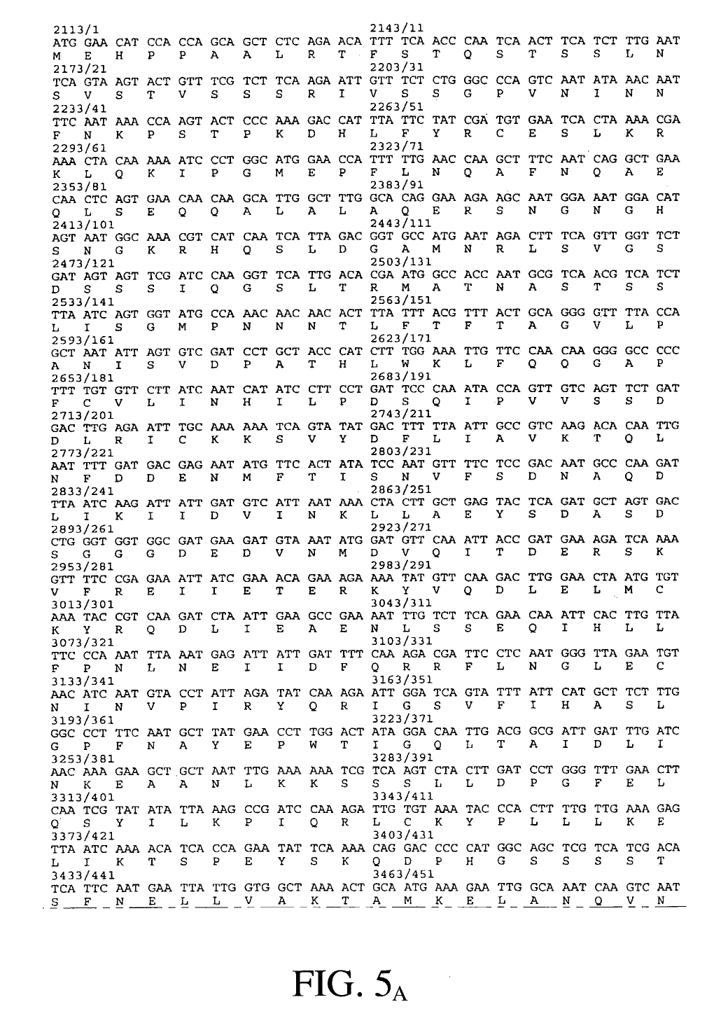

FIG. 7a which shows the percent sinilarity and percent homology for a BLAST line up of S. cerevisine Cdc24p (SEQ. I.D. No: 28) and C. abicans Cdc24p (SEQ. I.D. No: 29);

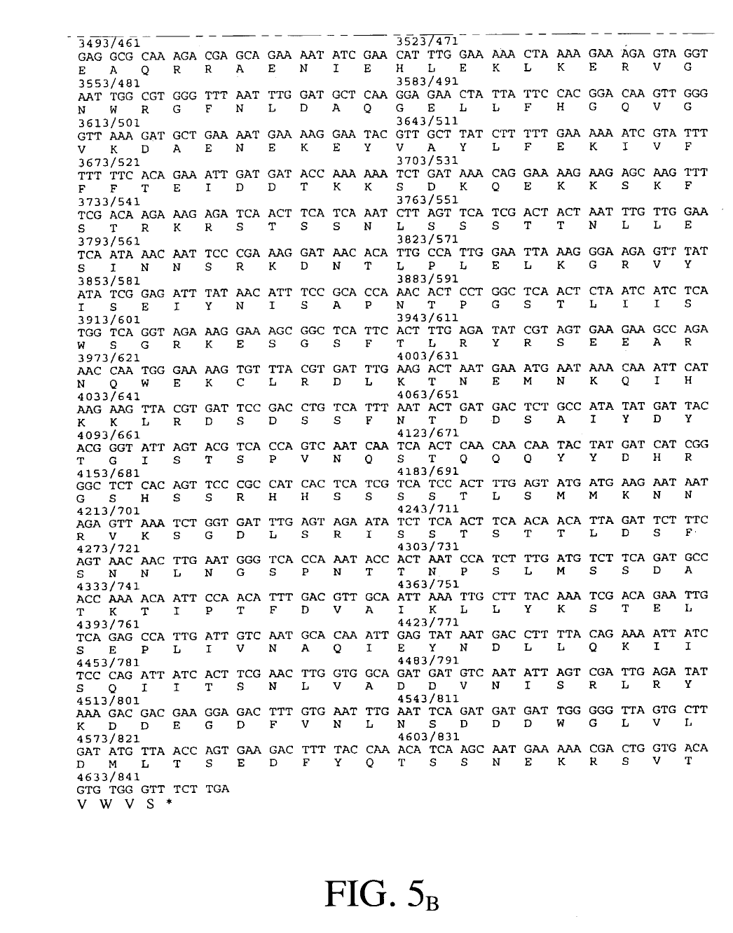

FIG. 7b which shows the percent sIfilarity and percent homology for a BLAST line up of S. cerevisiae Cdc24p (SEQ. I.D. No: 30) and S. pombe Cdc24p (SEQ. I.D. No: 31);

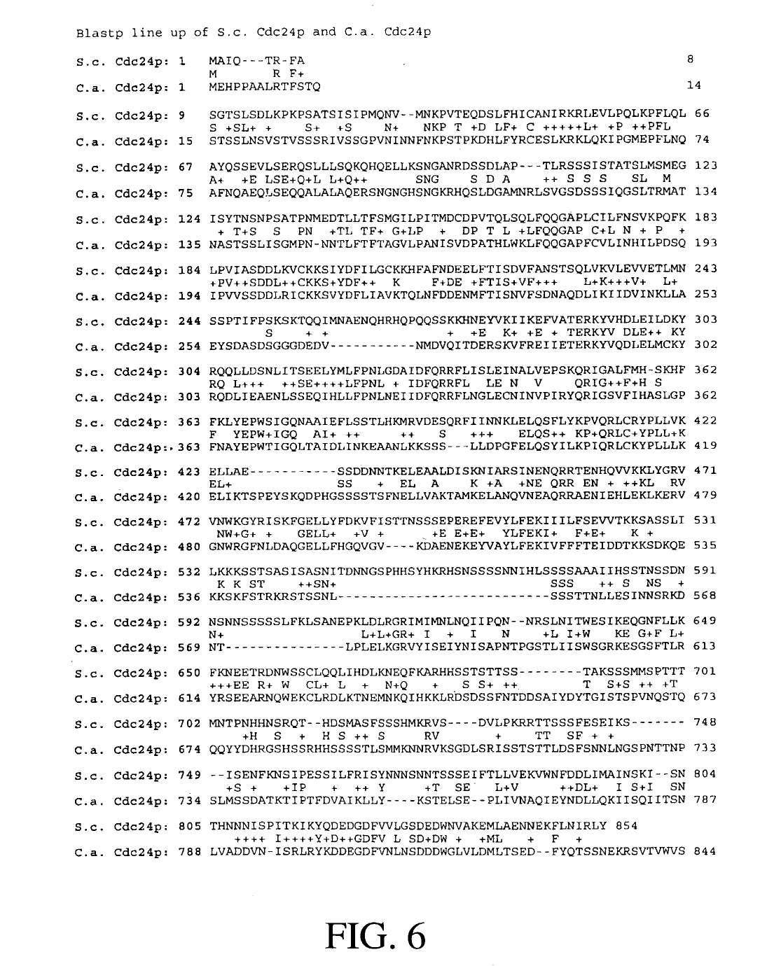

FIG. 8 which presents a comparison of the critical region (SEQ ID No 25) of S. cerevisiae Cdc24p with the corresponding sequence (SEQ ID No 26) in the C. albicans Cdc24p.

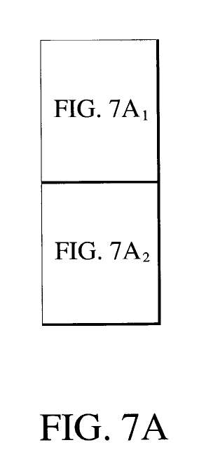

FIG. 9A whiclh presents a schematic illustration of C. albicans Cdc24p, showing that C. albicans Cdc24p is homologous to Cdc24p sequences fron K. lactis, S. pombe and S. cerevisiae.

FIG. 9B which shows a sequence alignment of the GEF domain of C. albicans Cdc24p with those of K. lactis, S. pombe and S. cerevrsiae.

FIG. 9C which shows that the N-terminal region required in S. cerevisiae for binding Ste4p is present in C. albicans and K. lactis and is similar in the S. pombe sequence.

FIG. 9D which shows that the C-terminus required for binding Bem1p in S. cerevisiae is homologous to C. albicalns, K. lactis and S. pombe Cdc24p sequences. The lines above sequence alignments indicate the residues involved in functional activity in S. cerevisiae Cdc24p. Sequence statistics are % identities and % positives generated by the BLAST aligorithin. Sequence alignments were performed using CLUSTALW V1.8.1

FIG. 10A which shows a schematic diagram of strain construction in C. albicans. A CDC24/cdc24 (PY12) strain was constructed by targetted gene replacement using a standard cassette in a wild type (PY1). This heterozygote was then used to construct a strain in which the remaining copy of CDC24 could be regulated by the MET3 promoter (MET3PCDC24/cdc24) (PY18). The MET3 promoter construct was made by cloning a BamHI/BgIII N-teminal fragment of CDC24 immediately downstream of the MET3 promoter in pCaDISCDC24. This plasmid is linearised by digestion at a unique Clal within the CDC24 sequence. Recomnbination places the endogenous copy of CDC24 immediately downstream of the MET3 promoter and leaves a small amino terminal fragment of CDC24 immediately downstream of the endogenous promoter. This construct also contains the URA3 marker between the CDC24 fragment and the MET3 promoter to facilitate selection on media.

FIG. 10B which shows PCR analysis of HISI knock-out. Schematic shows primer positions and expected sizes. CDC24/cdc24 genomie DNA tests positive for HISI gene at the CDC24 locus

FIG. 10C which shows PCR analysis of MET3 promoter knock-in. Schematic shows primer positions and expected sizes. MET3PCDC24/cdc24 mutant is positive for MET3 promoter immediately upstream of CDC24 START (pair 1 ) and negative for CDC24 promoter (pair 2). All samples are positive for the CDC24 sequence present in the upstream fragment of CDC24 (pair 3).

FIG. 11A which shows thatMET3PCDC24/cdc24 cells are inviable when grown on media containing methionine and cysteine. Bottom panel: Adding back a genomic copy of CDC24 is sufficient to rescue the growth defect of MET3PCDC24/cdc24 cells. Equal amoiits of cells were spotted on SC media lacking and containing methionine and cysteine (2.5 mM). Plates were incubated at 30° C. for 3/4 days.

FIG. 11B which shows that inviability in MET3PCDC24/cdc24 cells is due to the inability to bud in the presence of metionine and cysteine. Equal amounts of cells were grown in SC media lacking and containing methionine and cysteine (1.25 mM) and grown at 37° C. for 180 mins. At t=0, 60 180 mins. cell samples were fixed and percent cells with buds counted on a haemocytometer.

FIG. 12A which shows that CDC24 is required for invasive growth in C. albicans. Both the wildtype (CDC24/CDC24) and CDC24/cdc24 colonies invade agar equally well after 3 and 7 days MET3PCDC24/cdc24 cells are unable to invade agar even after 7 days.

FIG. 12B which shows that replacing a genomnic copy of CDC24 using an RP10 integration plasinid pCaEXPARG4CDC24 is sufficient to rescue the invasion phenotype of the MET3PCDC24/cdc24 colonies. I and II are independent his+ura+arg+ transformants from RP10 integration in MET3PCDC24/cdc24 cells. −CDC24 indicates cells were transformed with pCaEXPARG4 and +CDC24 indicates cells were transformed with pCaExPARG4CDC24.

FIG. 12C shows MET3PCDC24/cdc24 cells are unable to invade either YEPD or SC media containing FCS. Wildtype CDC24/CDC24 cells invade both media after 3 days. Comparing these images with those in panael A indicates that FCS and DFCS induce invasion similarly. Wildtype colony morphology is also dependent on media. YEPD+FCS induces crenilation whereas SC+FCS does not. Media lacking FCS does not alter colony morphology in wiidtype; colonies being round and smooth. MET3PCDC24/cdc24 colonies were never crenilated always being round and smooth after three days. All strains were grown in SC media overnight. Logarithmically growing cells were pelleted and resuspended in SC-met-cys and normalised for OD₆₀₀ to approximately 0.5. Then cells were plated on a 10 fold dilution series on YEPD+DFCS and incubated at 37° C. for time indicated. Colony invasion was visualised using a stereroscope with transmissive light at 50× on the days indicated. Scale bars represent 0.5 mm.

FIG. 13 which shows that CDC42 is required for invasive growth of C. albicans.

FIG. 14A which shows that after 3 hours in liquid YEPD media containing FCS at 37C wild-type cells Cabudl and CabemI display elongated germ tubes, with each many times the length of the cell body (FIG. 14A) whereas Cacdc24 and Cacdc42 cells appear to have little or no germ tubes.

FIG. 14B which shows a graph of the relative number of of cells with germ tubes in each of the cell types of FIG. 14A.

FIG. 15A shows CDC24 is required for germ tube formation in C. albicans in SC-met-cys+DFCS at 37° C. CDC24/CDC24 and CDC24/edc24 cells form germ tubes after 60 mins. MET3PCDC24/cdc24 are severely defected in germ tube formation after 180 mins in SC-met-cys+DFCS at 37° C. Scale bar represents 10 μm.

FIG. 15B which illustrates that MET3PCDC24/cdc24 cells show a 4-5 fold defect in germ tube formation after both 60 mins and 180 mins. Error bars represent maxima and minima of two independent determinations. Column height are means. In both A and B equal amounts of cells were incubated in SC-met-cys media containing DFCS (1:1) for 180 mins as per materials and methods. At t=0, 60, 180 mins cell samples were fixed and viewed at 60× magnification or percent germ tube formation was counted using a haemocytometer.

FIG. 16 which shows a schematic diagram of the possible position of CDC24 in morphogenetic signaling pathways of C. albicans. Serum seems able to activate morphogenesis by both the mating MAP kinase pathway and the cAMP dependant pathway. CDC24/CDC42 may signal via CST20 to activate the C. albicans dimorphic switch. Homologues of S. cerevisiae mating MAP kinase cascades have been placed in the relative positions by epistasis analysis

FIG. 17 shows various sequences of the invention.

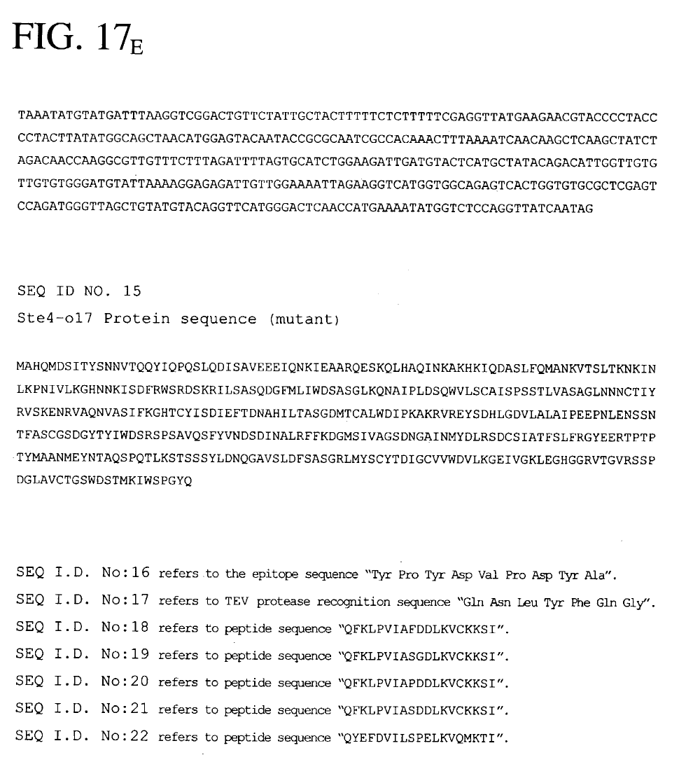

The Figures are discussed in more detail below.

### EXAMPLES

### SECTION A—S. cerevisine Examples

Materials and Methods

A1 General techniques

Strains were constrcted using standard technique²¹. All constructs were verified by DNA dye tenninator cycle sequencing (ABI377 sequencer).

Strains

pRS414CDC24 contains the CDC24 ORF including 258 bp upstream of ATG.

Oligonucleotide-directed mutagenesis was used to introduce silent base changes that resulted in the following ten new restriction sites in CDC24: NheI (bp-12), KasI (bp 283), AatII (bp 681), PstI (bp 1207), RsrII (bp 1369), BstEII (bp 1426), Xhol (bp 1758), MluI (bp 1963), SalI (bp 2061), BamHI (bp 2485). RAY410 (MATa, leu2, CDC24::LEU2, ade2, lys2, his3, trp1, ura3, pEG(KT)CDC24) was derived from the diploid YOC380²² which was transformed with pEG(KT)CDC24²³ and sporulated. RAY950 is isogenic to RAY410 but has pRS416GalHis₆CDC24 as a rescuing plasmid. RAY928 (MATa, leu2-3, 112, ura3-52, his3-D200, trp1-D901, lys2-801, suc2-D9, CDC24:;HIS5 pEG[KT]CDC24) and RAY931 (same as RAY928 but MATa, ade2, LYS2) were made by transformation of SEY6210 and 6211 with pEG(KT)CDC24 followed by PCR-based gene disruption of CDC24. The CDC24 ORF was replaced with S. pombe HIS5²⁴, flanked by LoxP sites. Replacement of CDC24 in SEY6211 with a PCR-generated integration cassette consisting of TRP1 fused to 343 bp of CDC24 promoter followed by 1704 bp of CDC24 or cdc24-m1 ORF was used to construct RAY1034 or RAY1035, respectively.

A2 IDENTIFICATION OF cdc24 MUTANTS WITH SPECIFIC DEFECTS IN CELL MATING:

A) Construction of a library of cdc24 random mutants

Error-prone PCR was used to generate a library of cdc24 mutants in a plasmid vector suitable for phenotypic screening in yeast.

1) Plasmid:

pRS414 CDC24 with upstream region and new restriction sites (referred to as pRS414CDC24).

2) Mutagenic PCRs:

Conditions from Fromant, M, Blanquet, S. & Plateau, P. Direct random mutagenesis of gene-sized DNA fragments using polymerase chain-reaction. Analytical Biochemistry 224, 347-353 (1995).

Different PCR-conditions were tested and the error-rate was determined by DNA sequencing. The following conditions were used for constructing the library used in the screen.

Composition of PCR-reactions (25 μl each):

DNA pRS414CDC24 600pM

| dATP | 0.23 | mM |
|---|---|---|
| dCTP | 0.20 | mM |
| dTTP | 2.9 | mM |
| dGTP | 0.42 | mM |
| Buffer | PCR Buffer supplied with | |
| | Taq-polymerase | |
| MgCl₂ | 4 | mM |
| MnCl₂ | 0.5 | mM |
| Taq (Ampli-Taq) | 2 | U per reaction |
| Primer: | 0.5 | mM |
| PCR-cycles: | | |
| step 1 94° C | 5 | min |
| step 2 91° C. | 1 | min |
| step 3 51° C. | 1 | min |
| step 4 72° C. | 3 | min |
| step 5 72° C. | 5 | min |

| | |
|---|---|
| step 6 4° C. | pause |
| 16 cycles (steps 2-4) | |

3) Library construction:

The PCR products were digested with AatII and NheI (680 bp corresponding to amino acid 1-227) were mutagenised and the resulting fragment ligated into pRS414CDC24 (cut with the same enzymes). Ligations were transformed into E. coli by electroporation and >50,000 transformants pooled for plasmid isolation.

A3 Phenotypic screening for cell-mating specific cdc24 alleles

Rationale:

To identify mutant cdc24 alleles which cause defects in cell mating but allow vegetative growth. Yeast strain RAY950, in which expression of CDC24 is repressed in glucose medium, was used.

1) Library plasmids were transformed into RAY950 and transformants selected on SC-trp plates which contained 2% glucose. As RAY950 does not grow on glucose plates this procedure eliminated all non-functional cdc24 mutants.

2) Transformants were replica-plated onto a lawn of WT (screen 1) or ΔfusIΔfus2 (screen 2) tester cells, incubated at 30° C. for 3 hrs and replica-plated onto plates selectng for diploids or RAY950 derived haploids. Mating defective mutants were identified by compariug the pattern of colonies on the two sets of plates and candidate mutants were picked from the original transfonnation plates for retesting.

3) Plasmids from mutants were isolated by transforrnation into E. coli. Isolated pasrnids were retransformed into RAY950, RAY928 and RAY931 for independent confirmation of phenotype and retested for defects in cell mating.

4) Mutations of confirmed mutants were identified by DNA sequencing. Multiple mutations were separated by subcloning and the mutation responsible for the phenotype identified by mating tests in RAY950.

5) A total of ˜5,000 yeast transfonnants were tested in each screen.

Screen 1 identified two mutants (cdc24-m1, cdc24-m2).

Screen 2 identified one mutant (cdc24-m3).

Phenotypic analyses

Quantitative matings¹⁰, matings in the presence of saturating pheromone13, halo-assays²⁶ using sst1::URA3 strains, and Fus11acZ measurements with pSG231¹¹ were carried out as described. Halo assays showed MATa and MATa cdc24-m1 cells secreted α-factor and α-factor, respectively. Actin was visualised with rhodamine phalloidin²⁷ on a Biorad-MRC-600 confocal microscope and pictures are projections of 4-6 0.5 mm z-series steps. For α-factor treatment cells were incubated with 5 mM α-factor for 2 hr. RAY1034 and RAY1035 cells were used to determine bud scar positions on zygotes¹⁴ visualised with Calcoflour²⁸. Similar results were observed wlth the position of the bud scar on shmoos. Direct measurement of cell orientation in a pheromone gradient was carried out essentially as described¹². A pheromone gradient was generated using a micropipet filled with 80 mM a-factor injected at 105 kPa into 1 ml of YEPD media layered on top of cells embedded in 2% Low Melting Point (LMP) agarose. Cells shape was recorded by video microscopy on a heated stage at 35° for 4-7 hr and data analysis was from traced cell outlines¹⁴. Mating projections were formed at the same pheromone concentrations and budding, that is non-responding cells were seen at similar distances from the micropipet in both strains.

Two-Hybrid methods

STE4, BEM1 (372-551 aa), CDC42[C178S], and CDC24/cdc24-m1 (1-288, 1-160, and 170-245 aa) were cloned by PCR into pGAD424 (AD, GAL4 activation domain) or pAS1 (DBD, GAL4DNA binding domain). Plasmids were transformed into HF7c. For determination of STE18 requirement, PCR-based geene disruption was carried out in PJ69-4A (MATa, trp1-901, leu2-3,112, ura3-52, his3-200, gal4D, gal80D, GAL2-ADE2, LYS2::GAL1-HIS3, met2::GAL7-lacZ)²⁹, replacing the entire STE18 ORF with K. Lactis URA3³⁰. For all two-hybrid experiments, equal amounts of transfonnants were spotted on SC-leu-trp and SC-leu-trp-his plates, identical results were obtained with at least four transformants, and for Dste18 two independent deletion strains. All strains for two-hybrid analyses expressed similar amounts of AD- and DBD- fusion proteins of the expected sizes, as determined by SDS-PAGE and immuno-blotting. None of the DBD fusions showed any self-activation using two different non-interacting AD fusions.

In vitro binding studies

A fragment of CDC24 (1-472 aa) in pGEX-2T (Pharmacia) and His₆Ste4p (pTrcSte4) were expressed in E. coli. Cells were resuspended in buffer A (PBS, 0.1% TX-100, Phenyl Methyl Sulfonyl Fluoride (PMSF), leupeptin, chymostatin, pepstatin, aprotinin) and lysed by snap freezing in liquid nitrogen followed by sonicatioin. Insoluble material was removed by centrifuigation (10,000 g). Mixed supernatants (denoted cell extracts) containing His₆Ste4 and GSTCdc24 fusions were incubated with GSH-agarose (Sigma Chemical Co.) at 4° for 1 hr. Resin was washed 3 times with buffer A. Resin samples (referred to as eluates) and extracts were analyzed by SDS-PAGE, immuno-blottinig probed with Omni-probe anti-sera (Santa Cruz), and visualised with enhanced chemiluhniuescence (Amershan). GSTCdc24p (1-127 aa), similar to GST, did not bind His₆Ste4p. Similar results were observed in 5 independent experiments.

A4 Ste4p mutants

Ste4p is the β-subunit of the heterodimeric G protein that can usually associate with Cdc24p exemplified by nucleotide SEQ. I.D. No: 10 and amino acid SEQ. I.D. No: 11. A mutation in STE4 exemplified by nucleotide SEQ. I.D. No:.12 and SEQ. I.D. No: 14 and amino acid SEQ. I.D. No: 13 and SEQ. I.D. No: 15 prevented the interaction of the mutant G protein β subunit with Cdc24p. Thus, it is possible to devise assays based on this mutation to screen for agents capable of modifying the non-interactive behaviour of the mutant G protein β subunit with Cdc24p. In addition, the assay could be used to study Cdc24p homologues or even Cdc24p derivatives or homologues to see if those derivatives or homologues affect the non-interactive behaviour of the mutant G protein β subunit.

The Ste4p mutants are also aspects of the present invention.

In this regard, the present invention also covers an STE4 mutant.

The present invention also covers a mutation of the β-subunit of the heterodimeric G protein that can usually associate with GEF (preferably Cdc24p) that is capable of preventing the interaction of the mutant G protein subunit with GEF (preferably Cdc24p)

Hence, a further aspect of the present invention is a mutation in STE4 - i e. on the β-subunit of the heterodirneric G protein that can usually associate with Cdc24p. This mutation prevents the interaction of the mutant G protein subunit with Cdc24p. Thus, likewise, it is possible to devise similar assays based on this mutation to screen for agents that modify the non-interactive behaviour of the mutant G protein with Cdc24p In addition, the assay could be used to study Cdc24p homologues or even Cdc24p derivatives or variants to see if those derivatives or variants affect the non-interactive behaviour of the mutant G protein. The sequences associated with this aspect of the present invention are shown as SEQ. I.D. Nos: 10-15. The present invention also covers variants or derivatives of such sequences—wherein the variants or derivatives of the wildtype sequences do not substantially affect Cdc24 interaction; and wherein the variants or derivatives of the mutant sequences do substantially affect Cdc24 interaction.

A5 Assay system to monitor the effects of two human oncogenic agents on an S. cerevisiae yeast mutant with a mating defect.

An assay system was devised to establish whether two different proto-oncogenes could complement the S. cerevisiae yeast phenotype (cdc24-m1) mating defect as described above and in Nern and Arkowitz (Nature (1998) 391: 195-198). The two oncogenic agents used were the human proto-oncogene, proto-Dbl and the mouse C4 protein which is almost identical to the human sequence, C5 Vav, and which is referred to hereafter as Vav. The S. cerevisiae cell division cycle molecule, Cdc24p, which is a protein with similiarities to proto-Dbl was used as a positive control in addition to the Cdc24p of the related yeast K. lactis.

Transgenic yeast organisms which co-expressed the nucleotide sequence (SEQ. I.D. No: 3) for the cdc24-m1 mating defect and the nucleotide sequence of interest (NOI) encoding either proto-Dbl, Vav or two related Cdc24p's were used.

The expression levels of the proto-oncogene, proto-Dbl, in S. cerevisiae were relatively low compared with the expression levels of the Cdc24p protein from either S. cerevisiae or K. lactis

Qualitatively, both proto-Dbl and K. lactis Cdc24 proteins parrially complemented the mating defect in the cdc24-m1 niutant. This result is in contrast to that obtained with the oncogenic form of Dbl alone which, although expressed, did not complement the cdc24-m1 mating defect. The Vav protein, did not display any effect on the mating defect. This lack of effect may be due to either insufficient expression of the Vav protein or to the fact that Vav function requires a pbosphoylation of the Lck kinase which must be co-expressed with the Vav protein before an effect can be observed.

A6 Assays to determine FAR1 interaction with Cdc24p and Gβ

Studies have shown that FAR1 may play an important role botb for pheromone mediated growth arrest and growth orientation during mating (Valtz, N., Peter; M. & Herskowitz, I. J. Cell Biol. 131, 863-73 (1995); Chang, F. & Herskowitz, I. Cell 63, 999-1011 (1990); Peter, M., Gartner, A., Horecka, J., Ammerer, G. & Herskowitz, I. Cell 73, 747-60 (1993)). The orientation fimction, which is specifically disrupted in a far1-H7 mutant, is required for the Cdc24 Gβ interaction suggesting that Far1 might interact with Cdc24. Two-hybrid analyses show that indeed Far1 interacts with Cdc24.

While the Cdc24 Gβ interaction requires the presence of FAR1, the Far1 Cdc24 interaction is independent of Gβ, suggesting that Far1 might bind Cdc24 directly whereas Cdc24 Gβ are part of a complex which include Far1. Far1 also interacts by two-hybrid assays with Gβ, consistent with the notion that Cdc24, Far1, and Gβ form a complex. In a diploid two-hybrid strain, in which a number of pheromone response genes are not expressed, we are unable to detect the Cdc24 Gβ interaction. However, overexpression of Far1 results in an interaction and furter overexpression of Gγ results in a maximal interaction, indicating that a complex comprised of Cdc24, Gβγ, and Far1 forms even in diploid cells.

Although cdc24-m and far1-s mutants result in similar defects in growth orientation during mating, we set out to determine if these genes function in the same orientation process. Generation of a cdc24-m1 mutation in a Δfar1 strain did not result in a substantial decrease in mating efficiency, suggesting these two genes function in the same process. In contrast, results from double mutants of cdc24-mI with Δspa2, Δste20, or Δbem1 suggest that these three genes do not function in the same orientation process as Cdc24 and Far1. Cdc24 and Far1 were epitope tagged in order to determine whether these proteins interact in yeast cells. The chronosomal copy of Cdc24 was replaced with a 3×myc tagged Cdc24 and the chromosomal copy of Far1 was replaced with Far1 protein A fusion. Both of these fusion proteins are fully functional. Isolation of Far1-protein A from yeast extracts using IgG-Sepharose co-precipitated 3×myc-Cdc24. In contrast, the 3×myc-Cdc24-m1 mutant was defective in binding Far1 in similar imnmunoprecipitation assays. These results indicate that Cdc24 and Far1 bind one-another and this interaction may be essential for growth orientation during mating.

A7 Far1 binds Cdc24 and Gβ

The binding relationships between Cdc24, Far1, and Gβ were examined in vitro using proteins purified from bacteria and yeast. Gβγ was purified from yeast cells using a chromosomal copy of the gene which has HA epitope (Tyr-Pro-Tyr-Asp-Val-Pro-Asp-Tyr-Ala) fused to the amino-terminus and protein A fused to the carboxyl-tennius. A tobacco etch virus (TEV) protease cleavage site (recognition site Glu-Asn-Leu-Tyr-Phe-Gln-Gly with cleavage occurring between Gln and Gly) was placed between Gβ and the protein A domain so that material isolated from yeast using IgG-Sepharose can be specifically eluted with commercially available recombinant TEV protease. Maltose binding protein (MBP) Far1 fusions have been expressed and purified from E. coli. Similarly, a glutatliione-S-transferase (GST Cdc24 fusion (residues 1-472) has been expressed and purified from E. coli. MBP-Far1 binds GST-Cdc24 specifically. The removal of the 75 carboxyl-termirni residues of Far1 (H7) prevents Cdc24 binding. Furtermore GST alone is unable to bind MBP-Far1.

These results show that Cdc24 can directly bind Far1 in the absence of any other yeast proteins. Far1 fragments containing either the amino-terminal Lim domain (a domain implicated in protein-protein interactions) or the carboxyl-tenrmius were tested for their ability to bind GST-Cdc24. Both fragments showed very little binding to GST-Cdc24 indicating that although the Far1 carboxyl-terminus is necessary, it is not sufficient for Cdc24 binding. Using MBP-Far1 we have been able to observe binding to Gβ purified from yeast. Binding of Gβ is reduced using amino-ternal or carboxy-termuial MBP-Far1 fragments, yet Gβ binds Far1H7 as well as Far1.

In one preferred asepct of the present invention the assay also includes the presence of Far1.

A8 RESULTS

TABLE 1
cdc24-ml is defective in cell mating

| Strain | Tester | % Mating efficiency |
|---|---|---|
| CDC24 MATα | MATa WT | 100 (21) |
| cdc24-ml MATα | MATa WT | 0.5 (0.2) |
| CDC24 MATa | MATa WT | 100 (20) |
| edc24-ml MATa | MATa WT | 3.8 (1.6) |
| CDC24 MATa | MATα Δfus1 Δfus2 | 100 (17) |
| cdc24-ml MATa | MATα Δfus1 Δfus2 | ≦0.02 |
| CDC24 MATa | CDC24 MATα | 100 (18) |
| cdc24-ml MATa | cdc24-m1 MATα | ≦0.0006 |

Mating efficiencies are the number of diploid cells divided by the total cells with CDC24 WT set to 100%. The values are means of 4 determinations with standard deviation &Ovalhollow;. Absolute mating efficiency was 14-15% with MATa and MATα testers, 1.8% with Δfus1 Δfus2 tester, and 3.4% with CDC24 tester.

Some of the results are also shown in the accompanying Figures. These Figures are now discussed in more detail.

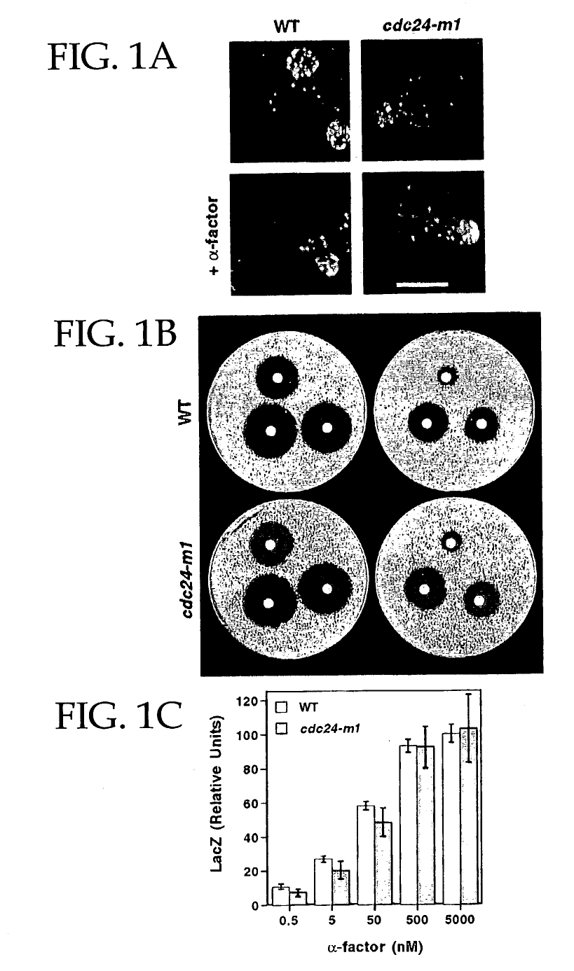

FIGURE. 1

cdc24-m1 phenotypes. a, Actin cytoskeleton of cdc24-m1 cells shows polarised distribution. Bar equals 5 mm. b, Pheromone-induced growth arrest is similar in cdc24-m1 with WT cells. Sterile filter disks spotted with α-factor (1, 0.5, 0.2, 0.1, 0.05, and 0.012 mg) were placed onto cells in agarose. c, MAP-kinase pathway signalling is unaffected in cdc24-m1. LacZ activities are the average of 2 experiments (2-3 detenminations per experiment) with standard deviation. WT maximum (29.6 Miller Units) was set to 100%.

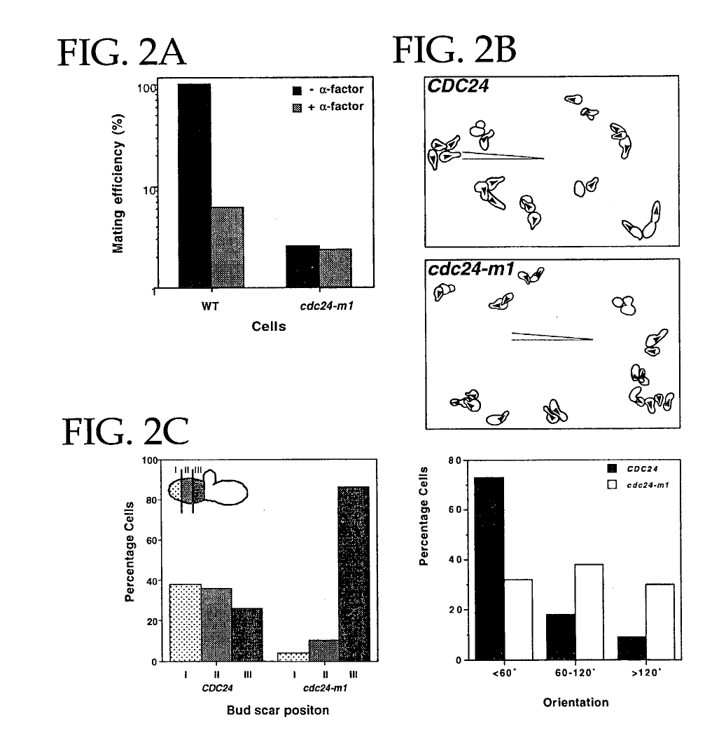

FIGURE. 2

cdc24-m1 cells are unable to orient in a pheromone gradient. a, Excess pheromone has a negligible effect on cdc24-mI mating. MATa cells were mated with a WT tester and mating efficiency for CDC24 (2.8%) was set to 100%. Values are means (n=2). b, cdc24-m1 cells are unable to orient in a pheromone gradient. A trace of cell shapes after 6-7 hr in a pheromone gradient is shown with arrowheads indicating orientation. Quanititadon of cell projection angle relative to the micropipet (needle) from 4-7 separate experiments (n=112 CDC24 and 167 cdc24-m1 cells). The average cosine of the angle of cell projection relative to the micropipet was 0.52 for CDC24 and −0.02 for cdc24-m1 cells (a cosine of 1 represents perfect orientation and 0, random orientation) c, cdc24-m1 cells position their shinoos adjacent their bud scar. The position of the bud scar on zygotes was determined for approximately 120 cells.

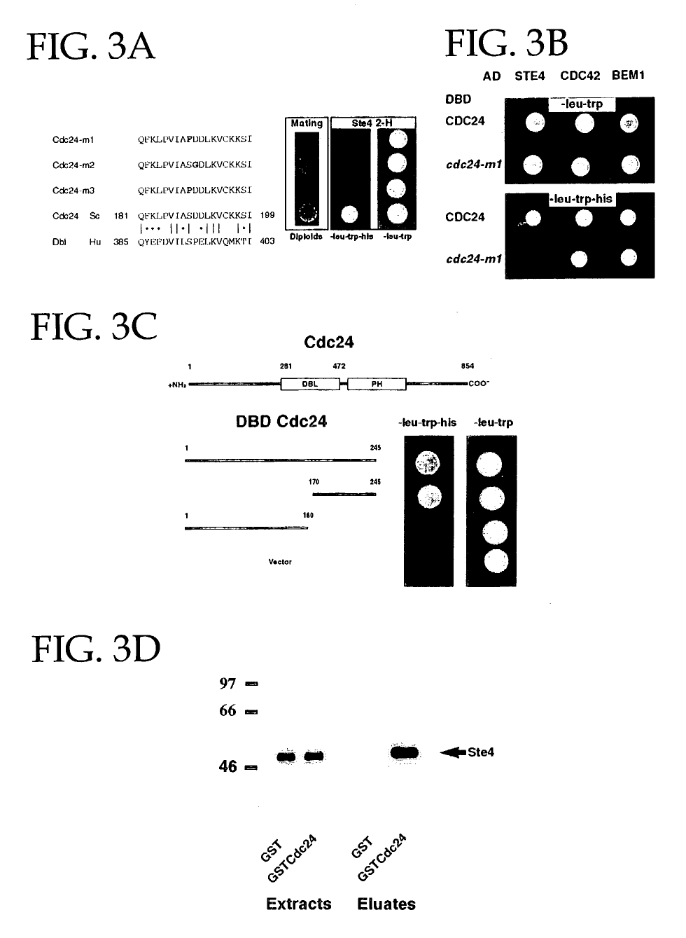

FIGURE. 3

cdc24-m mutants are defective in mating and Ste4p (Gβ) binding. a, Location of Cdc24p mating mutations. Mating patches show diploids from mating with MATa WT tester. Ste4 2-H patch growth on -leu-trp-his indicates an interaction of Cdc24p (1-288 aa) with Ste4p. Similar results were obtained using a LacZ reporter in strain Y187 (relative Miller Units 100 for Cdc24/Ste4 and 3 for Cdc24-m1/Ste4). b, Two hybrid interactions of Cdc24p. Fror interactions with Ste4p, a fragment of Cdc24p (1-288 aa) was used, however, fill length Cdc24p also interats with Ste4p. c, Region of Cdc24p necessary for Ste4p interaction. Numbers refer to Cdc24p aa fused to DBD. d, Cdc24p binds to Ste4p in the absence of other yeast proteins. Mixed bacterial cell extracts (1 eq) containing either His₆Ste4p and GST or GSTCdc24p (1-472 aa), and GSH-agarose eluates (800 eq) were separated by SDS-PAGE, immuno-blotted and probed with anti-sera to His₆Ste4p. Anti-CST sera showed simiar amounts of GST and GSTCdc24p in eluates. Due to proteolysis, His₆Ste4p migrates as a doublet.

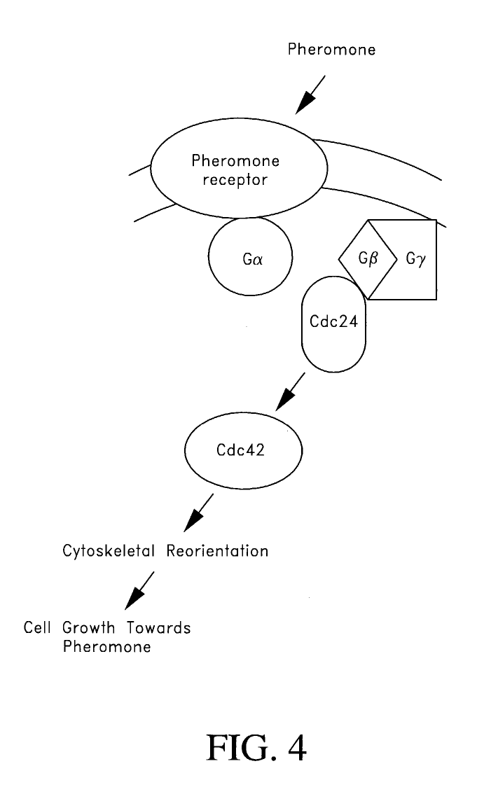

FIGURE. 4

Model for signal transduction pathway required for cell orientation. For clarity we have omitted components of MAP-kinase cascade. The role of Cdc42p (a Rho-family GTPase) in cell orientation is speculative. Pheromone binds the pheromone receptor (Ste2p or Ste3p) resulting in the dissociation of Gα (Gpa1p) from Gβγ (Ste4p/Ste18p). Direct binding of Cdc24p to Gβγ (in the vicinity of the receptor) activates or recruits Cdc42p which is necessary for oriented growth towards a mating partner.

SEQUENCE ANALYSIS

The DH and PH sequences were analysed by a Blast homology search In addition, an analysis of the amino acid identity over the entire protein to S. cerevvicae Cdc24p was conducted. DH refers to the Dbl homology region (GEF region)—see Hart et al 1991 Nature 354 311-314; Miyamoto et al 1991 Biochem Biophys Res Commun 181 604-610; Ron et al 1991 New Biol 3 372-379 PH refers to the Pleeksin homology region—see Musacchio et al Trends Biochem Sci 18 343-348.

The results are as follows:

A. Blast homology search using Cdc24 DH and PH region TBLASTN 1.4.9 MP

Query=yeast Cdc24p DH PH (392 aa):

| | |
|---|---|
| KIIKEFVATERKYVHDLEILDKYRQQLLDSNLITSEELYMLFPNLGDAIDFQRRFL | (SEQ. I.D. NO:9) |
| ISLEINALVEPSKQRIGALFMHSKHFFKLYEPWSIGQNAAIEFLSSTLHKMRVDE | |
| SQRFIINNKLELQSFLYKPVQRLCRYPLLVKELLAESSDDNNTKELEAALDISKNI | |
| ARSINENQRRTENHQVVKKLYGRVVNWKGYRISKFGELLYFDKVFISTTNSSSE | |
| PEREFEVYLFEKITILFSEVVTKKSASSLILKKKSSTSASISASNITDNNGSPHHSYH | |
| KRHSNSSSSNNIHLSSSSAAAIIHSSTNSSDNNSNNSSSSSLFKLSANEPKLDLRG | |
| RIMIMNLNQIIPQNNRSLNITWESIKEQGNFLLKFKNEETRDNWSSCLQQLIHDL | |
| KN | |

Database. Non-redundant Genbank+EMBL+DDBJ+PDB sequences 349,525 sequences; 540,957,745 total letters

Reference: Altschul, Stephen F., Warren Gish, Webb Miller, Eugene W. Myers, and David J. Lipmnan (1990). Basic local alignment search tool. J. Mol. Biol. 215;403-410.

| | | | | Smallest | Smallest |
|---|---|---|---|---|---|
| | | | | Sum Prob- | Sum Prob- |
| | | Reading | High | ability | ability |
| | | Frame | Score | P(N) | N |
| gb\|U12538\|SPU12538 | Schizosaccharomyces | +3 | 171 | 1.0e−51 | 6 |
| | pombe scd1 | | | | |
| emb\|X57298\|MMMCF2PO | M. musculus Mcf2 | +1 | 128 | 8.3e−10 | 3 |
| | proto-oncogene | | | | |
| | (Mcf2 is Dbl) | | | | |
| gb\|U16296\|HSU16296 | Human T-lymphoma | +3 | 88 | 2.3e−09 | 3 |
| | invasion and metastasis | | | | |
| | inducing TIAM1 | | | | |
| gb\|U05245\|MMU05245 | Mus musculus BALB/c | +3 | 88 | 5.5e−09 | 3 |
| | invasion inducing | | | | |
| | protein (Tiam-1) | | | | |
| gb\|J03639\|HUMDBLTP | Human DBL oncogene | +2 | 121 | 2.1e−07 | 3 |
| | encoding a transforming | | | | |
| | protein | | | | |
| gb\|S76992\|S76992 | VAV2 = VAV oncogene | +3 | 125 | 2.6e−07 | 2 |
| | homolog human | | | | |
| dbj\|D86547\|D86547 | Fruitfly still life type 1 | +2 | 76 | 5.4e−07 | 5 |
| gb\|U37017\|MMU37017 | Mus musculus Vav2 | +1 | 126 | 6.4e−07 | 2 |
| | oncogene | | | | |
| dbj\|D86546\|D86546 | Fruitfly still life type 2 | +1 | 76 | 1.0e−06 | 5 |
| gb\|U39476\|RNU39476 | Rattus norvegicus p95 | +3 | 116 | 6.3e−06 | 1 |
| | Vav proto-oncogene | | | | |
| gb\|S76838\|S76838 | Obs (Dbl guanine | +3 | 112 | 4.4e−05 | 2 |
| | nucleotide exchange | | | | |
| | factor homolog) murine | | | | |
| dbj\|AB002360\|AB002360 | Human KIAA0362 | +2 | 113 | 4.5e−05 | 2 |
| emb\|Z35654\|RNOSTOG | R. norvegicus Ost | +1 | 112 | 4.9e−05 | 2 |
| | oncogene | | | | |
| emb\|X83931\|HSVAVONCO | H. sapiens VAV | +1 | 109 | 5.5e−05 | 1 |
| | oncogene | | | | |
| gb\|AF003147\|CELC11D9 | Caenorhabditis elegans | +3 | 81 | 0.0070 | 3 |
| | C11D9 | | | | |
| gb\|U96634\|MMU96634 | Mus musculus p85SPR | +2 | 62 | 0.016 | 3 |
| emb\|Y10159\|DDY10159 | D. discoideum racGAP | +1 | 71 | 0.025 | 3 |
| gb\|U58203\|MMU58203 | Mus musculus Lsc | +2 | 75 | 0.044 | 2 |
| | oncogene | | | | |
| emb\|Y09160\|HSSUB15 | H. sapiens Sub1.5 | +1 | 80 | 0.063 | 2 |
| gb\|AF003740\|CELC41D11 | Caenorhabditis elegans | +2 | 81 | 0.064 | 4 |
| | C41D11 | | | | |
| gb\|U02081\|HSU02081 | Human guanine | +1 | 77 | 0.12 | 2 |
| | nucleotide regulatory | | | | |
| | protein (NET1) | | | | |
| gb\|U00055\|CELR02F2 | Caenorhabditis elegans | +1 | 85 | 0.13 | 1 |
| | R02F2 | | | | |
| gb\|U64105\|HSU64105 | Human guanine | +1 | 77 | 0.14 | 1 |
| | nucleotide exchange | | | | |
| | factor p115-RhoGEF | | | | |
| gb\|U42390\|HSU42390 | Homo sapiens Trio | +1 | 74 | 0.33 | 3 |
| gb\|M24603\|HUMBCRD | Human bcr protein | +1 | 58 | 0.91 | 3 |
| | amino cnd | | | | |
| emb\|X02596\|HSBCRR | Human bcr (breakpoint | +3 | 58 | 0.996 | 3 |
| | cluster region) in | | | | |
| | Philadelphia | | | | |
| | chromosome | | | | |
| gb\|U11690\|HSU11690 | Human faciogenital | +2 | 73 | 0.999 | 1 |
| | dysplasia (FGD1) | | | | |
| gb\|U22325\|MMU22325 | Mus musculus | +3 | 73 | 0.9997 | 2 |
| | faciogenital dysplasia | | | | |
| | (Fgd1) | | | | |
| gb\|M15025\|HUMBCRABL | Human BCR/ABL | +3 | 58 | 0.99995 | 5 |
| | product of the | | | | |
| | translocation of | | | | |
| | t(222q11; 9q34) | | | | |

B. Amino acid identity over entire protein to S. eerevisiae Cdc24p

| | | protein | % identity |
|---|---|---|---|
| Organism | gene | size (aa) | (aa) |
| Schizosaceharomyces pombe | Scd1 | 834 | 21.9 |
| Mouse | Fgd1 | 960 | 16.7 |
| Human | Fgd1 | 961 | 16.5 |
| Mouse | Vav2 | 868 | 16.5 |
| Mouse | Ect2 | 768 | 16.2 |
| Human | Vav2 | 878 | 15.8 |
| Worm | Q18479 | 860 | 15.4 |
| Mouse | Vav | 844 | 14.6 |
| Rat | Vav | 843 | 14.5 |
| Human | Vav | 846 | 14.4 |
| Mouse | Dbs | 1150 | 14.3 |
| Human | Tim | 519 | 14.0 |
| Human | proto-Dbl | 925 | 13.4 |
| Human | p115RhoGEF | 912 | 13.4 |
| Mouse | Lfc | 572 | 13.4 |
| Rat | Ost | 872 | 12.9 |
| Worm | Q22354 | 862 | 12.9 |
| Mouse | Lsc | 919 | 12.5 |
| Human | Lbc | 424 | 12.4 |
| Human | Net1 | 460 | 12.3 |
| Human | BCR | 1271 | 11.9 |
| Mouse | Tiam1 | 1591 | 11.2 |
| Human | Tiam1 | 1591 | 10.9 |
| Mouse | proto-Dbl | 320 | 9.7 |
| | | (partial) | |
| Drosophila | Still Life 1 | 2064 | 9.0 |
| Drosophila | Still Life 2 | 2044 | 8.4 |

| | |
|---|---|
| Protein name key; | |
| Scd1: | Schizosaccharomyce pombe Cdc24p¹⁰¹. |
| Fgd1 | Faciogenital Dysplasia Protein. FGD also known as |
| | Aarskog-Scott syndrome, is an X-linked develop- |
| | mental disorder¹⁰². |
| Vav/Vav2 | A oncogene derived from hematopoietic cells¹⁰³. |
| Q18479 | (similar to Vav) |
| Q22354 | (similar to Vav) |
| Ect2 | Oncogene expressed in epithelial cells and possessing |
| | transforming potential¹⁰⁴. |
| Tim | Mammary epithelial oncogene¹⁰⁵. |
| Dbl/Dbs | Diffuse b-cell lymphoma (dbl) oncogene^{106, 107}. |
| p115RhoGEF | Regulates cell proliferation, induces the transformation |
| | of cells¹⁰⁸. |
| Lfc | Hematopoietic oncogene¹⁰⁹. |
| Ost | Osteosarcoma derived proto-oncogene. Truncation is |
| | oncogenic and highly tumorigenic in mice¹¹⁰. |
| Lsc | Oncoprotein¹¹¹. |
| Lbc | Oncogene involved in chronic myeloid leukemias¹¹². |
| Net1 | Neuroepithelioma transforming oncogene¹¹³. |
| BCR | bcr (breakpoint cluster region), an oncogene which is |
| | the translocation breakpoint in chronic myeloid |
| | leukemias (CML)^{114, 115}. |
| Tiam1 | Human invasion- and metastasis-inducing tiam1 gene |
| | and is expressed in tumor-cell lines of different tissue |
| | origin¹¹⁶. |
| Still Life 1/2 | A synaptic terminal protein¹¹⁷. |

A9 DISCUSSION—Section A

CDC42 and its GDP/GTP exchange factor (GEF) CDC24 are required for vegetative growth^{8,9} and cell mating^{6,7,10}. The precise fliction of these proteins in cell matig has been difficult to study because they are essential for viability. In accordance with the present invention, we reasoned that if CDC24 has a specific ftnction in the mating pathway, cdc24 alleles should exist which affect cell mating but not vegetative growth. To identify such alleles, a collection of CDC24 random mutants was screened and three recessive mating mutants, cdc24-m1-3 were isolated (FIG. 3A). This screen required isolated cdc24 mutants to be able to support vegetative growth. Further characterisation of cdc24-m cells revealed normal growth between 18° and 37° and cell morphology, bud site selection, and actin distribution were similar to WT cells (see below and FIG. 1A). The specificity of the cdc24-m phenotype is in contrast to that of all other described cdc24 mutants which have strong defects in vegetative growth⁸⁻¹⁰.

To elucidate the role of CDC24 in mating, we examied cdc24-m1 cells for defects in the mating pathway. The mating efficiency of cdc24-m1 cells with a WT partner was reduced approximately 100-fold compared to WT (Table 1), and this effect was essentially independent of mating type. When cdc24-m1 or an enfeebled mater defective in cell fusion were used as mating parters, significantly stronger defects were observed. Such bilateral mating defects suggest impairment in a process such as shmoo (mating projection) formation, orientation, or fusion in which a WT matng partner can partially compensate for the mutant strain.

Pheromone activation results in a number of responses including cell cycle arrest, MAP-kinase cascade mediated induction of mating specific genes, and changes in cell morphology^{4,5}. Pheromone-induced growth arrest determined by halo-assays showed both cdc24-m1 and WT cells responded similarly (FIG. 1B). Furthermore, overexpression of the β-subunit of the yeast hetero-trimeric G-protein, Ste4p, frpm an inducible promoter arrested growth of both cdc24-m1 and WT cells (data not shown). Microscopic examination revealed identical nunbers of WT and cdc24-m1 cells (78%, n=1600) formed shmoos after 4 hr exposure to 10 mM pheromone. The actin distribution of cdc24-m1 budding and shmooing cells was also similar to that of WT cells (FIG. 1A), demonstrating that tle mating defect was not due to an inability to polarise the actin cytoskeleton. The level of pheromone induced FUS1-lacZ expression, a reporter used to measure induction of mating specific genes¹¹, was similar in cdc24-m1 and WT cells (FIG. 1C). However, exanon of mating mixtures of cdc24-m1 and WT tester cells showed a greater than ten-fold decrease in the number of zygotes, indicating that the cdc24-m1 defect occurs prior to cell fusion. Thus cdc24-m cells appear normal for cell cycle arrest, shmoo formation, actin cytoskeleton polarisation, and MAP-kinase signalling, yet are defective at a step prior to cell fusion.

During mating, polarised growth towards a mating partner requires a pheromone gradient¹² and saturation wit pheromone during mating results in random orientation of growth and mating parner selection, and hence a decrease in mating efficiency^{13,14}. WT cells showed a 16-fold decrease in mating efficiency in the presence of saturating pheromone (20 mM), whereas only 10% reduction was observed with cdc24-m1 cells (FIG. 2A), suggesting that this mutant is unable to orientate towards a pheromone gradient during mating. Similar results were observed with cdc24-m2 and cdc24-m3 cells. To test directly whether cdc24-mI cells are defective in mating projection orientation their response to an artificial pheromone gradient created by a micropipet was examined. While CDC24 cells oriented growdt towards the pheromone source (greater than 70% of cells oriented within 60° angle of micropipet), cdc24-m1 cells did not show a preferred orientation (FIG. 2B). No difference in the sensitivity of WT or mutant cells to pheromone was observed.

Although cdc24-m1 cells oriented randomly in a pheromone gradient, the choice of shmoo site could bc dictated by an internal cue, such as the previous bud site. To examine this possibility, the location of the bud scar (in cells with a single bud scar) relative to the neck of the zygote was determined. While WT cells showed a random position of their bud scar on the zygotes, 86% of cdc24-m1 zygotes had formed a shrnoo adjacent to their previous bud site (FIG. 2C). Together these results establish a specific role for Cdc24p in orientation towards a mating partner.

Sequencing of cdc24-m alleles revealed mutations that changed one of two adjacent amino acid residues (FIG. 3A). cdc24-m1 and cdc24-m3 both have a single amino acid change from Ser 189 to either a Phe or Pro. cdc24-m2 had two amino acid substitutions and subeloning demonstrated that the mutation responsible for the mating defect is Asp to Gly at residue 190. The grouping of these mutations suggests that this region of Cdc24p is important for an interaction required for oriented growth.

Previous two-hybrid studies have suggested that the amino-tenius of Cdc24p might interact with Ste4p⁷, however, the in vivo significance of this association was unclear. We determined whether Cdc24p mating mutants could interact with Ste4p (FIG. 3B). In contrast to the wild-type Cdc24p, the mutants did not show a detectable interaction with Ste4p. In agreement with the clustering of the cdc24-m mutations, amino acid residues 170 to 245 of Cdc24p were sufficient for the Ste⁴p interaction (FIG. 3C), while an amino-tenninal fragment consisting of the first 160 amino acid residues, although expressed, failed to interact. Consistent with a functional significance of the Cdc24p Ste4p interaction, we have isolated mutants in STE4, (exemplified by SEQ. I.D. No: 10 and SEQ. I.D. No: 11), using a two-hybrid screen, which are unable to interact with Cdc24p and are phenotypically sinilar to cdc24-m mutants.

To assess the specificity of the defect in the interaction between Ste4p and Cdc24-m1p, interactions with Cdc42p and Bem1p, two proteins known to bind to Cdc24p^{15,16} were investigated. Bem1p is an SH3 domain protein involved in bud formation and mating¹⁷. Cdc24-m1p was able to interact with both Cdc42p and Bem1p (FIG. 3B) consistent with the absence of an effect of cdc24-m1 on vegetative growth.

While the cdc24-m1 phenotype along with the two-hybrid results indicates that the interaction between Cdc24p and Gβ is central to cell orientation, these results do not address whether this interaction is direct or indirect. Gβ typically fuinctiors as a complex with the third subunit of a hetero-trimeric G-protein, Gγ. We therefore determined whether the yeast Gγ, Ste18p, was required for the Cdc24p Ste4p interaction. Deletion of STE18 abolished the Cdc24p Ste4p two-hybrid interaction (data not shown), suggesting that Cdc24p interacts with the Gβγ-complex. To determine if Cdc24p could directly bind Ste4p, these proteins were expressed in bacteria. Hexahistidine-tagged Ste4p specifically bound to GSTCdc24p (FIG. 3D). These results demonstrate tat Cdc24p can directly bind Gβ in the absence of any other yeast proteins. We attribute the requirement for Gγ in the two-hybrid assays to its stabilisation of Gβ¹⁸.

Pheromone receptor activation results in dissociation of Gβγ from Gα at the receptor Our results indicate that the orientation defect in cdc24-m cells is due to a specific defect in the Cdc24p Gβγ interaction. This suggests a model in which direct binding of Cdc24p to Gβγ results in recruitment (to the vicinity of the receptor) or activation of Cdc42p and that this local concentration of activated Cdc42p is responsible for oriented growth towards a pheromone gradient (FIG. 4). In the absence of his recruitment or activation a site adjacent to the previous bud site appears to function as a default site for shmoo formation. Our results together with previous studies implicating Cdc24p in bud site selection⁸, suggest that Cdc24p acts as a crucial component required both for bud and shmoo site selection, perhaps functioning as a kind of molecular selector switch between internal signals for bud site selection and external signals for shmoo site selection. It is likely that local activation of Cdc24p recruits and activates the Rho GTPase Cdc42p, which could then interact with downstream targets required for orientation of the cytoskeleton. Cdc42p interactions with the protein kinase Ste20p^{19,20} are not necessary for cell orientation²⁰, suggesting that novel targets of Cdc42p are required for oriented growth towards a mating partner.

Cdc24p belongs to a diverse family of GEFs which include many mammalian proto-oncogenes². This group of proteins shares a conserved region consisting of a Dbl-domain (named after the human proto-oncogene Dbl) followed by a plecktstrin-homology domain (PH). Sequence comparison revealed similarity between a small stretch of amino acids flanking the cdc24 mating mutations and Dbl (FIG. 3A). Our results indicate that an association between Cdc24p and Gβγ liks pheromone receptor activation to shmoo orientation. We propose that other GEFs, such as the proto-oneogene Dbl, provide a similar connection between G-protein coupled receptor activation and polarised cell growth.

Hence, in accordance with the present invention tere are provided the following uses and utilities of Cdc24p/Ste4 interaction and cdc24m mutants

1) Peptide inhibitors which block GEF/Gβ interaction. Peptides and peptidyl derivatives based regions encompassing mutants will be used to block and/or antagonise GEF (such as the proto-oncogenes Dbl or Vav) Gβ interaction. Derivatives of these peptides (including peptide minimics) which bind with higher affinity will also be used The perturbation of these interactions will be of therapeutic value for example in treatnent of cancers.

2) Simple yeast based assays systems (utilising mating function and interaction reporters) will be extremely useful for high ftrough-put screening to identify molecules perturbing this GEF/Gβ interaction. In particular, the qualitative effect on mating observed with the proto-oncogene, proto-Dbl, even at low levels of expression, indicates that this type of assay is amenable to large scale screening for the effect of agents, such as proto-oncogenes, on induced defects in yeast and other host cells.

3) Similar Cdc24p/Gβ interactions will be ideal targets for anti-fingal drags directed at the pathogenic yeast Candida, as shown in the Section B of the Examples.

### SECTION B—C. albicans Examples

CDC24 is a key regulator of the Candida albicans dimorphic switch

Candida albicans is a dimorphic fimgal pathogen of humans (Odds, 1988). Like other yeasts it reproduces vegetatively by budding but, upon exposure to environmental cues, switches its growth pattern to produce germ tubes, extend hyphae and become invasive. Switching between a budding and invasive hyphal fonn is thought to be important for virulence of Candida albicans (Cutler, 1991). Morphological changes such as budding and hyphae formation require incorporation of cell wall material at discrete sites on the cell surfacc; a process termed polarised growth. Studies of morphological changes in the yeast Saccharomyces cerevisiae have shown that once a growth site has been selected the actin cytoskeleton polarises to deliver vesicles containing new cell wall material (Adams and Pringle, 1984; Kilmartin and Adams, 1984; Baba et al; 1989; Read et al, 1992). S. cerevisae polarises its growth at two points in its life cycle; once during budding and again when haploid cells respond to mating pheromone secreted from a cell of the opposite mating type (mating). As shown above, in both these processes a key link between the growth site and the actin cytoskeleton is the guanine nucleotide exchange factor (GEF) Cdc24p and its small GTPase Cdc42p. Bot these proteins are essential and temperature sensitive mutants of both arrest as round unbudded cells at non-permissive temperatures (Stoat et al, 1978; 1981; Cdc42p reviewed Johnson, 1999). These cells exhibit delocalised deposition of chitin presumably caused by the inability to polarised their actin cytoskeleton. As described above, Cdc24p localises to sites of polarised growth; either the budsite or the site of mating phermone receptor activation. Once localised to these sites it is thought to locally activate the G-protein Cdc42p that in turn activates transcription of mating specific genes (Simon et al, 1995) and direct changes to the actin cytoskeleton allowing polarised secretion and growtdh.

In Candida albicans two sigalling pathways, defined by the transcription factors Cph1p and Efg1p, are involved in triggering the dimorphic switch (Brown and Gow, 1999). Δefg1Δcph1 double mutants are unable to make the yeast/hyphal switch whereas single mutants retain some ability to forrm hyphae (Lo et al, 1997). The Egf1p pathway appears to be mediated by cAMP. Presently this pathway remains largely obscure beyond the observations that exogenous cAMP induces switching and serum (in liquid medium) is able to induce hyphal growth in an Efg1p dependant manner (Lo et al, 1997). The Cph1p pathway is comprised of C. albicans homologues of elements of the mating MAP kinase pathway in Saccharomyces cerevisiae. Cph1p itself is homologous to the transcription factor Ste12p (Lui et al, 1994), the kinases Cst20p, Hst7p and Cek1p are C. albicans homologues of Ste20p, Ste7p and Kss1p respectively. Furthernore, epistastic analysis demonstrated they occupy the same relative positions in the pathway as their S. cerevisiae homologues (Köhler and Fink, 1996; Leberer et al, 1 996; Whiteway et al, 1992; Brown and Gow, 1999; Csank et al, 1998). While many inducers of dimorphic switching are recognised no receptors or ultimate targets have been identified for either pathway. There are however examples of hyphal specific genes and genes whose regulation differs in budding and hyphal formation; one such gene is the C. albicans homologue of Cdc42p The rate of accumulation of CDC42 transcript slows during hyphal formation (Mirbod, et al, 1997) but the significance of this observation has yet to be addressed.

Other yeasts grow in a filamentous fashion. Following nitrogen starvation diploid Saccharomyces cerevisiae cells become pseudohyphal and invade solid surfaces. Pseudohyphae result from repeated rounds of wholeell elongation and unipolar division; these elongated cells remain joined together producing invasive filaments (Gimeno et al, 1994). In contrast, C. albicans hyphae result from highly focused growth at a particular point on the cell periphery producing byperpolarised cells with a distinct cell body bearing narrow hyphae many times longer tan the originating cell. Other non-morphological differences between the invasive growth of these two yeasts exist. Candida albicans switches in response to a greater variety of stimuli, including serum (Barlow et al, 1974), temperature, neutral pH and growth on rich media but responds only modestly to nitrogen starvation—the main trigger of pseudohyphal growth in S. cerevisiae. However, genetic analysis of the signalling pathways showed that homologous pathways regulate pseudohyphal growth in S. cerevisiae and C. albicans; these being elements of the mating MAP kinase pathway (Ste20p/Ste7p/Ste11p and Ste12p) and a cAMP/protein kinase A pathway (Brown and Gow, 1999; Liu et al, 1993) More interestingly, signalling through the MAP kinase pathway via Cdc42p/Ste20p is required to induce filamentation in nitrogen starved S. cerevisiae (Mösch et al, 1996). Thus, Cdc24 and/or Cdc42 (exchange factor and/or GTPase) are used recurrently to control all morphological changes that occur during budding, mating and pseudohyphal formation in S. cerevisae. In particular, as disclosed by the present application, Cdc24p, by virtue of its polarised localisation, provides a landmark of polarised growth. and locally activates Cdc42p. Signalling via Cdc42p results in transcriptional responses to environmental stimuli (Zhao et al, 1995) and polarisation of the actin cytoskeleton.

In the examples detailed below, we test if Cdc24p could be a regulator of dimorphic switching in C albicans. Using the MET3 promoter (Care et al, 1999), we demonstrate that the C. abicans Cdc24p is essential due to its role in bud formation. In repressive conditions mutants arrest as round unbudded cells. Surprisingly, constitutive expression of Cdc24p causes a specific defective of invasion and hyphal formation. Thus, Cdc24p appears to have a specific role in switching to or maintaining polarised growth states in Candida albicans. We also show that Cdc42p also has aln important role in switching. We therefore propose that the Cdc24p/Cdc42p module is a key regulator of the C. albicans dimorphic switch.

Materils and Methods

B1 Media and Strains

YEPD+uridine (referred to a YEPD (Yeast extract peptone dextrose) media contained 11 g yeast extract, 22 g bactopeptone, 55 mg adenine sulphate, 22 g agar, 80 mg uriidine, 20 g glucose per litre. Synthetic complete (SC) media contained 8 g Difco yeast nitrogen base without amino acids, 55 mg adenine sulphate, 55 mg tyrosine, 80 mg uridine, 20 g agar and 20 g glucose per litre. Amino acids were added as necessary for auxotrophic requirements. Liquid media contained no agar. For agar invasion assays and germ tube fornation foetal calf serum (FCS)(PAA laboratories, Austria) or dialysed FCS (DFCS) was added 1:1 to either 1× liquid or 2× solid media. Candida albicans and Saccharomyces cerevisiae strains used are described in Tables II and III.

Table II
Candida albicans strains.

| Strain | Genotype | | Reference |
|---|---|---|---|
| PY1 | UrU3Δ::λimm434/ura3Δ::λimm434 | his1::hisG/his1::hisG | Wilson et al. |
| | arg4::hisG/arg4::hisG | | (1999) |
| PY12 | Ura3Δ::λimm434/ura3Δ::λimm434 | his1::hisG/his1::hisG | This study |
| | arg4::hisG/arg4::hisG cdc24Δ::HIS1/CDC24 | | |
| PY18 | Ura3Δ::λimm434/ura3Δ::λimm434 | his1::hisG/his1::hisG | This study |
| | arg4::hisG/arg4::hisG cdc24Δ::HIS1/Met3PCDC24 | | |
| PY30 | Ura3Δ::λimm434/ura3Δ::λimm434 | his1::hisG/his1::hisG | This study |
| | arg4::hisG/arg4::hisG cdc24Δ::HIS1/Met3PCDC24 RP10::ARG4/RP10 | | |
| PY31 | Ura3Δ::λimm434/ura3Δ::λimm434 | his1::hisG/his1::hiSG | This study |
| | arg4;:hisG/arg4::hisG cdc24Δ:HIS1/Met3PCDC24 RP10::ARG4/RP10 | | |
| PY32 | Ura3Δ::λimm434/ura3Δ::λimm434 | his1::hisG/his1::hisG | This study |
| | arg4::hisG/arg4::hisG cdc24α::HIS1/Met3PCDC24 RP10::ARG4- | | |
| | CDC24/RP10 | | |
| PY33 | Ura360 ::λimm434/ura3Δ::λimm434 | his1::hisC/his1::hisG | This study |
| | arg4::hisG/arg4::hisG cdc24Δ::HIS1/Met3PCDC24 RP10::ARG4- | | |
| | CDC24/RP10 | | |

Table II
Saccharomyces cerevisiae strains.

| Strain | Genotype | Plasmid | Reference |
|---|---|---|---|
| RAY 1042 | MATα, leu2, cdc24::LEU2, ade2, lys2, his3, | pRS414 CDC24 | Nern and |
| | trp1, ura3 | | Arkowitz, 1998 |
| RAY 1044 | MATα, leu2, cdc24::LEU2, ade2, lys2, his3, | pRS414 cdc24-m1 | Nern and |
| | trp1, ura3 | | Arkowitz, 1998 |
| RAY 234 | MATα, his4-34, leu2-3, 112, ura3-52, fus1-Δ1, | | Nern and |
| | fus2-Δ3 | | Arkowitz, 1998 |
| RAY 876 | MATα, leu2-3, 112, ura3-52, his3-Δ200, | pRS406 | Nern and |
| | trp1-Δ901, ade2, suc2-Δ9 | | Arkowitz, 1998 |
| 70-2 | MATα, leu2, cdc24::LEU2, ade2, lys2, his3, | pRS404 cdc24-ts | Arkowitz lab. |
| | trp1, ura3 | | collection |
| 112-2 | MATα, leu2, cdc24::LEU2, ade2, lys2, his3, | pRS404 cdc24-ts | Arkowitz lab. |
| | trp1, ura3 | | collection |
| 14D3 | MATα, leu2, cdc24::LEU2, ade2, lys2, his3, | pRS406 cdc24-ts | Arkowitz lab. |
| | trp1, ura3 | | collection |

B2 Plasnids and Cloning

Plasmnids were constructed by standard techniques and are described in Table IV.

The C. albicans gene encoding the CDC24 protein was cloned in Bluescript as a 5.162 Kb genomic DNA fragment from KpnI to NsiI into KpnI PstI of Bluescript This cloned C. albicans CDC24 includes 1.95 Kb upstream of the CDC24 ATG start codon and 0.683 Kb downstream of TGA stop codon.

Degenerate primers were based on sequence similarities between S. cerevisiae and K. lactis CDC24 (the latter gene which we have cloned (Nern & Arkowitz unpublished)).

Forward Primer (SEQ. I.D. No: 32):

5′-AAR TAY RTK CAN GAY TTR GA -3′

Where

R=A or G

Y=C or T

K=G or T

N=A, C, G, or T

Reverse Primer (SEQ. I.D. No: 33):

5′-RAT TTT YTC RAA NAR RTA-3′

Where

R=A or G

Y=C or T

K=G or T

N=A, C, G, or T

C. albicans CDC24 was initially identified using the above degenerate primers and PCR (polyrnerase chain reaction) from a Candida albicans geniomic DNA libiar in a multicopy 2-micron S. cerevisiac URA3 vector (Liu, H. P., Kohler, J., and Fink, G. R. [1994] Science 266, 1723-1726)- (pRS202 vector (gift from G. Fink)). These primers were used to screen this library first as a pool of DNA in which we tried several degenerate primer pairs and finally on single bacterial library tansformants. Exact match oligonucleotides were then used to amnplify a 1-kB region between these two sequences and identify a library plasmid (pB2) containing this 1-kb sequence. The clone was then sequenced and we used several different exact match C. albicans CDC24 primers based upon this sequence to get the entire gene. Sequencing this plasmid revealed a 3.4-kB insert which encoded tile first 444 amino acids of C. albicans Cdc24p. The carboxyl-terminus of C. albicans CDC24 was isolated by PCR using an exact match oligonucleotide to C. albicans CDC24 and an oligonucleotide (M13F) to the library vector. This 2.9-kb PCR product was cloned into a pCR2.1-TOPO vector (Invitrogen following manufactures instructions) resulting in pTOPO2. The entire CDC24 including promoter and terminator was constructed by ligation of a 3.0-kB Kpn1/Ppuml fragment from pB2 and a 1.9-kB PpumI/NsiI fragment from pTOPO2 into a KpnI/PstI digested pBluescript vector yielding pBSCaCDC24.

Table IV
Plasmids used in this study.

| Plasmid name | Description | Reference |
|---|---|---|
| pGEMHIS1 | Previously described | Wilson et al, 1999 |
| pRSARG4ΔSpeT | Previously described | Wilson et al, 1999 |
| PCaDIS | Previously described | Care et al, 1999 |
| PCaEXP | Previously described | Care at al, 1999 |
| pB2 | pRS202 vector with genomic fragment of CaCDC24 | This study |
| | encoding amino terminal 444 amino acids | |
| pTOPO2 | Cloned PCR product encoding carboxy terminal 400 | This study |
| | amino acids of CaCDC24 | |
| pBSCaCDC24 | Full length CaCDC24 in pBluescript vector | This study |
| pCaDISCDC24 | pCaDIS wit a BamHI/Bg411 fragment of CaCDC24 | This study |
| PCaEXPARG4 | pCaEXP with ARG4 marker in place of URA3 marker | This study |
| PCaEXPARG4CDC24 | pCaEXPARG4 with a genomic copy of CDC24 in | This study |
| | place of MET3 promoter | |
| pCacdc24::HIS1 | Plasmid cut with SwaI/NotI to release targened gene | This study |
| | replacement cassette for CaCDC24. | |
| p2ATPIHACaCDC24 | Multicopy 2μ ADE2 plasmid with a triose phosphate | This study |
| | isomerase (TPI) promoter and haemagglutinin (HA) | |
| | epitope tag 5′ of C. albicans CDC24 | |
| p2ATPIHAScCDC24 | Multicopy 2μ ADE2 plasmid with a triose phosphate | Arkowitz Lab. |
| | isomerase (TPI) promoter and baemaggludnip (HA) | collection |
| | epitope tag 5′ of S. cerevisiaeCDC24 | |
| p2ATPIHA | Multicopy 2μ ADE2 plassnid with a triose phosphate | Arkowitz Lab |
| | isomerase (TPI) promoter and haemagglurinin (HA) | collection |
| | epitope tag | |

C. albicans homologues of Saccharomyces cerevisiae CDC42, BUD1, and BEM1 genes were also isolated. BUD1 and BEM1 encode for a Ras-like C-protein necessary bud site selection and an SH3 domain containing cytoskeleton associated scaffolding protein, respectively. The CaCDC42, CaBUD1, and CaBEM1 genes were isolated by suppression of a Saccharomyces cerevisiae cdc24 temperature sensitive mutant.

Gene knock-out of C. albicans CDC24 was carried out using a pCacdc24::HIS1 cassette made by ligation of a 2.0-kB NaeI/SwaI fragment from pGEM-HIS1 into a 5.3-kB HpaI/MscI digested pBSCaCDC24, yielding pCacdc24::HIS1 in which only the 21 carboxy-terminal amino acid residues remain. For C. albicans CDC24 gene replacement, this cassette was cut with SwaI/NotI prior to transforrnation. Methionine regulated expression of C. albicans Cdc24p was accomplished by cloning a 5′ fragment of CDC24 into pCaDIS using a BamHI site placed imnediately 5′ of the CDC24 ATG codon by PCR and a BgIII site within CDC24 resulting in pCaDISCDC24. This plasmid was integrated into the genomic copy of CDC24 in PY12 by cutting with ClaI resulting in integration of the MET3 promoter (MET3P) immediately 5′ of the CDC24 ORF. For integration of an additional copy of CDC24, pCaEXP was used in which the URA3 marker was replaced with C. albicans ARG4 (pCaEXPARG4). A 5.2-kB HpaII/BamHI CDC24 fragment from pBSCaCDC24 was then ligated into a BamHI/NarI cut pCaEXPARG4 which removed the MET3P resulting in pCaEXPARG4CDC24. For C. albicans CDC24 expression in S. cerevisiae p2ATPIHACaCDC24 was used.

B3 Sequencing, assembly and comparison of DNA sequences

Sequencing was done using the DNA dye terminator method. Sequences were assembled and edited using Seqman software. Sequence comparison and identification was done with the BLAST algorithm. Alignments were produced using ClustalW v1.8.1.

B4 Yeast transformations

Transformation of Candida albicans was as described (Wilson et al, 1999). Cells were plated on appropriate selective media and transformants confirmed by PCR from genomic DNA. Transformation of S. cerevisiae was by standard techniques (Rose et al, 1991).

B5 Functional analysis in S. cerevisiae

The function of C. albicans CDC24 was tested by putting it in a S. cerevisiae yeast vector (using for example a 2 micron vector with a triose phosphate isomerase promoter from S. cerevisiae to drive expression) and looking for complementation of different S. cerevisiae cdc24 temperature sensitive mutants and also cdc24-m mating mutants in S. cerevisiae. A recent paper that has tested and shown functionality of a C. albicans gene in S. cerevisiae is: R. S. Care, J. Trevethick; K. M. Binley; and Sudbery, P. E. (1999). The MET3 promoter: a new tool for Candida albicans molecular genetics, Molecular Microbiology 34, 792-798.

For growth complementation assays S. cerevisiae cdc24 temperature-sensitive strains (70-2, 112-2 and 14D3) transformed with p2ATPIHA, p2ATPIHAScCDC24 or p2ATPIHACaCDC24 were plated out in a ten-fold dilution series on selective media with and without 1M sorbitol and grown at 25, 30, 34 and 37° C. for 3 days.

For mating assays strain RAY1042 and RAY1044 transformned with the same plasmids were spotted onto YEPD, mixed wth either RAY234 or RAY876, and incubated at 37° C. for 4 hours. The plates were then replicated onto diploid selective plates. For immanoblotting, stationary cultures were back-diluted and grown for 5 hours. Ten ml of cells were pelleted and broken by glass bead lysis in breaking buffer (150 mM NaCl, 50 mM Tris HCl pH 7.4 1 mM PMSF, 40 μg/mL each of leupeptin, chymostatin, pepstatin A, aprotinin, and antipain) at 4° C. Extracts were analyzed by SDS-PAGE, blotted onto nitrocellulose membrane, and probed with anti-HA mAB (12CA5, 1:40 tissue culture supernatent) followed by HRP-goat-anti-mouse secondary (1:1000) and visualisation by ECL. As a loading control immunoblots were subsequently probed with an α-TCM1 monoclonal antibody.

B6 Cell wall chitin staining

C. albicans cells fixed in 4% formaldehyde were pelleted and stained with Calcofluor white (4 μg/mL) for 5 mins. After washing 5 times in PBS cells were visualised by fluorescence microscopy at 100× magnification on a Ziess Axioscope using a standad DAPI filter set.

B7 Viability and invasion assays

To assess viability of C. albicans mutants strain CDC24/CDC24 (PY1), CDC24/cdc24Δ (PY12) and MET3PCDC24/cdc24Δ (PY18) were grown to logarithmic phase in SC, pelleted, and resuspended in SC-met-cys and normalised for OD₆₀₀≈0.5/ml. Cultures were spotted in a 10 fold dilution series on SC−met−cys or SC+met+cys (2.5 mM) and grown for 3 days at 30° C. For invasion assays, 100-fold dilution series was spotted on YEPD, YEPD+DFCS, YEPD+FCS or SC+FCS and grown for 13 days at 30° C. and 37° C. Colonies were visualised at 50× magnification using a Leica stereoscope. Images were recorded using a CCD cannera

B8 Germ tube assays

Stains (PY1, PY12 and PY18) were grown in SC at 30° C. Equal amounts of cells were pelleted and resuspended in SC−met−cys or SC+met+cys and equal volumes of either DFCS or water were added (final concentration of met and cys was 1.25 mM). After 0, 60 and 180 min. cells were fixed in 4% formaldehyde for 1 hr. Cell numbers were counted and images taken using a CCD camera. To assess viability after 180 min. 100 μL of a 1:1000 and 1:10,000 dilution of unfixed cells were plated on SC−met−cys and grown at 30° C.

Results

B9 Cloning of Candida albicans CDC24 and sequence comparison

To isolate the C. albicans CDC24 we screened a genomic library by PCR. Degenerate oligonucleotides were based on DNA encoding conserved amino acids in the Cdc24p sequences from Schizosaccharomyyces pombe (Chang et al., 1994), Kluyvermlyces lactis (Nern & Arkowitz unpublished), and Saccharomyces cerevisiae guaine nucleotide excge factor (GEF) and pleckstrin homology (PH) domains. Two initial sequences, largely of the C. albicans GEF domain, were obtained by sequencing of PCR amplified regions. Subsequent screening using exact match oligonucleotides was successful in identifying one partial genomic clone (pB2) of CDC24 encoding the amino terminal 444 residues. This clone included both coding and promoter sequence. Te remaining portion, encoding the carboxy terminal 400 residues, was cloned and sequenced from a PCR product. Three independent clones of this PCR product were sequenced. Full lengtlh CDC24 was constructed by subcloning the two halves (pBSCaCDC24).

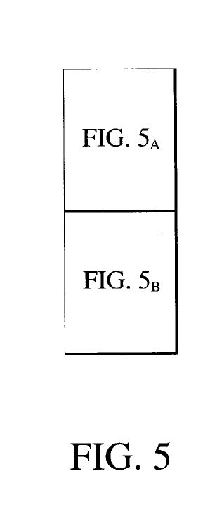

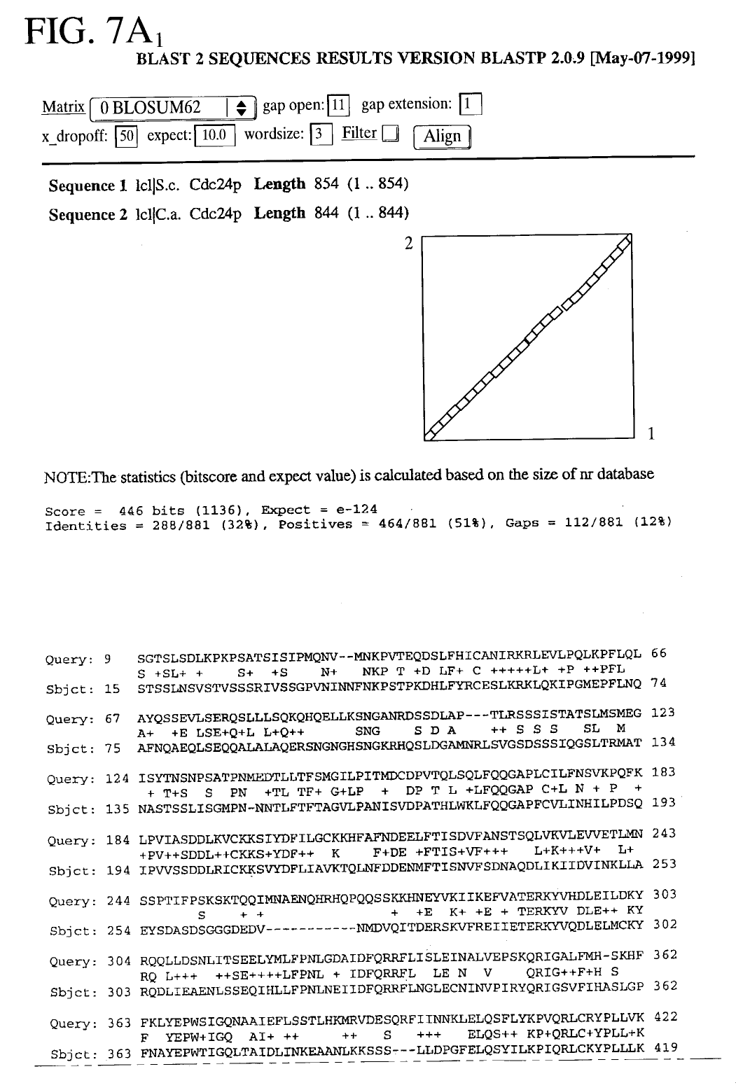

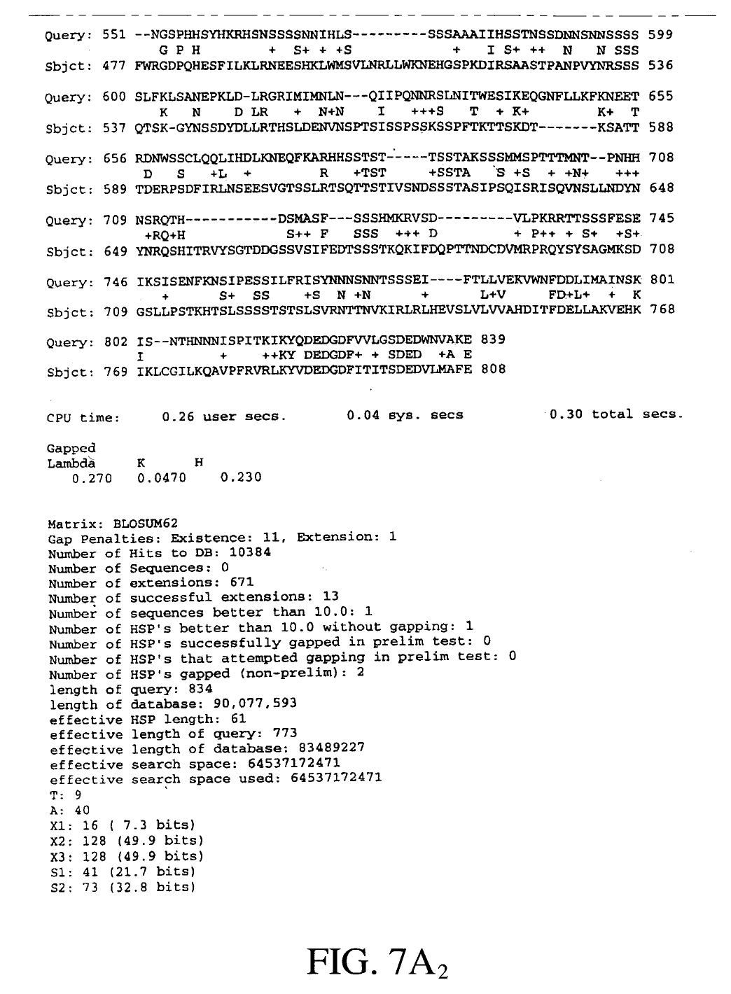

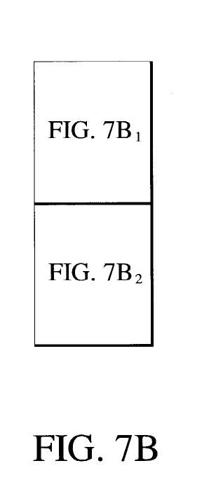

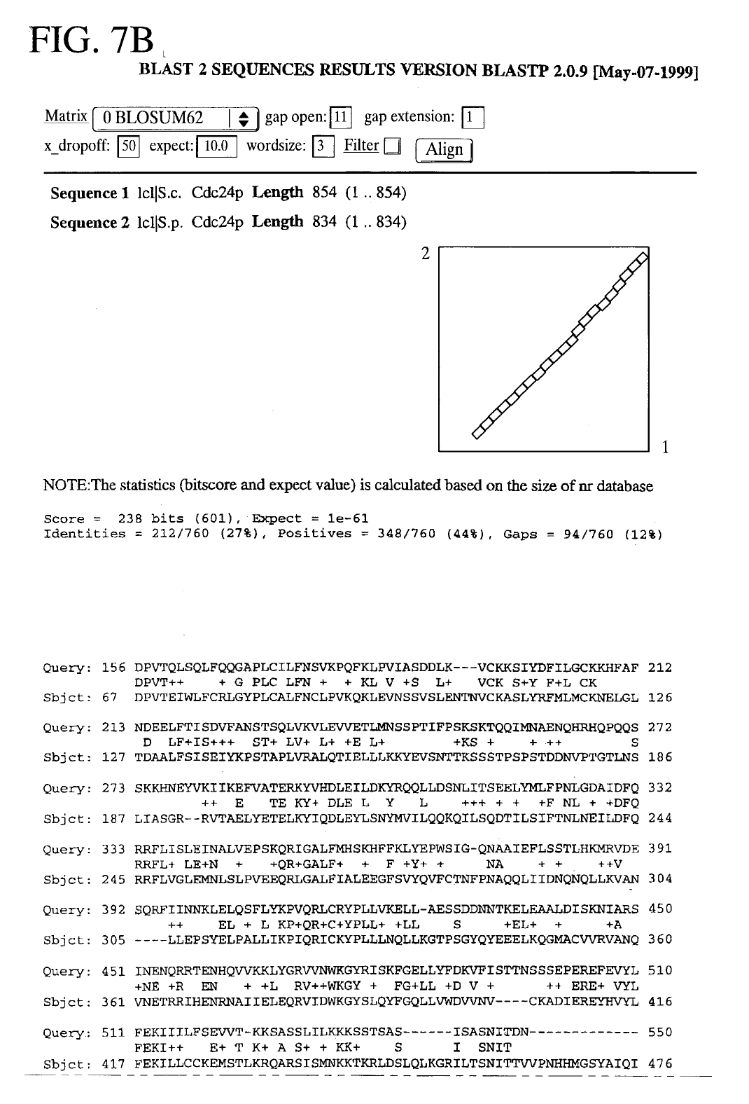

Candida albicans CDC24 encodes an 844 amino acid protein (FIG. 5) of 94.8 kD with signifcant homology to other fingal Cdc24p's which themselves form part of a larger class of proteins with homology to the human oncogene DBL (Cerione and Zheng, 1996) C. albicans Cdc24p is 32% identical and 51% homologous to S. cerevisiae Cdc24p (FIG. 9A). The GEF domain of the C. albicans protein in 43% identical and 62% homologous to S. cerevisiae GEF domain (FIG. 9B). As described above, Ste4p (Gβ) binds Cdc24p between amino acids 170 and 245 in S. cerevisiae. Bem1p binds the carboxy terminal 76 amino acids of Cdc24p (Zheng et al, 1995). Both these regions of C. albicans Cdc24p are homologous to other fingal Cdc24p's (FIGS. 9C and 9D). As shown below, the 19 amino acid fragment of C. albicans (SEQ. I.D. No: 35) corresponding to the 19 amino acid piece of the S. cerevisiae Cdc24p with sinilarity to the human proto-oncogene Dbl shares 89.5% homology with the corresponding S. cerevisiae Cdc24p 19 amino acid fragmeent (SEQ. I.D. No: 21) and the 76 amino acid fragment of C. albicans (SEQ. I.D. No: 34) corresponding to amino acids 170 and 245 in S. cerevisiae (SEQ. I.D. No: 1) shares 75.0% homology with the corresponding S. cerevisiae 76 amino acid fragment, A 73 arnino acid frament of C. albicans (corresponding to amino acids 170 to 242 in S. cerevisiae(SEQ. I.D. No: 37)) shares 75.3% homology with the corresponding S. cerevisiae fragment.

19 amno acid piece

| | | | | |
|---|---|---|---|---|
| 1 | QFKLPVIASDDLKVCKKSI | 19 | Sc | (SEQ. I.D. No: 21) |
| | ++PV++SDDL++CKKS+ | | | |
| 1 | DSQIPVVSSDDLRICKKSV | 19 | Ca | (SEQ. I.D. No: 35) |

% Identity=52.6 (10/19)

% Similarity=36.8 (7/19)

% Similarity+Identity=89.5 (17/19)

73 amino acid piece (SEQ. I.D. No 37 (S. cerevisiae) SEQ. I.D. No: 36 (C. albicans)

| | |
|---|---|
| 1 | PLCILFNSVKPQFKLPVIASDDLKVCKKSIYDFILGCKKHFAFNDEELFTISDVFANSTSQLVKVLEVVETLM |
| | P C+L N +P ++PV++SDDL++SKKS+YDF++ K F+DE +FTIS+VF+++ L+K+++V+ L+ |
| 1 | PFCVLINHILPDSQIPVVSSDDLRICKKSVYDFLIAVKTQLNFDDENMFTISNVFSDNAQDLIKIIDVINKLL |

% Identity=43.8 (32/73)

% Similarity=31.5 (23/73)

% Similarity+Identity=75.3 (55/73)

76 amino acid piece (SEQ. I.D. No: 1 (S. cerevisiae) SEQ. I.D. No: 34 (C. albicans))

| | |
|---|---|
| 1 | PLCILFNSVKPQFKLPVIASDDLKVCKKSIYDFILGCKKHFAFNDEELFTISDVFANSTSQLVKVLEVVETLMN |
| | P C+L+N+ P ++PV++SDDL++CKKS+YDF++ K F+DE +FTIS+VF+++ L+K+++V+ L+ |
| 1 | PFCVLINHILPDSQIPVVSSDDLRICKKSVYDFLIAVKTQLNFDDENMFTISNVFSDNAQDLIKIIDVINKLLA |

% Identity=42.1 (32/76)

% Similarity=30.3 (23/76)

% Similarity+Identity=72.4 (55/76)

However, when G/A and T/A are considered to be similar, the similarity of the 76 amino acid fragnent increases to 75.0%.

Numbers and Lineup for 76 arniino acid piece (where G/A and T/A similar residues are shown on middle line by a *.

76 amino acid piece (SEQ. I.D. No: 1 (S. cerevisiae), SEQ. I.D. No: 34 (C. albicans))

| | |
|---|---|
| 1 | PLCILFNSVKPQFKLPVIASDDLKVCKKSIYDFILGCKKHFAFNDEELFTISDVFANSTSQLVKVLEVVETLMNSS |
| | P C+L N + P ++PV++SDDL++CKKS+YDF++* K F+DE +FTIS+VF+++* L+K+++V+ L+ |
| 1 | PFCVLINHILPDSQIPVVSSDDLRICKKSVYDFLIAVKTQLNFDDENMFTISNVFSDNAQDLIKIIDVINKLLAEY |

% Identity=42.1 (32/76)

% Similarity=2.9 (25/76)

% Similarity+Identity=75.0 (57/76)

Therefore, we would predict that C. albicans Cdc24p is a guanine nucleotide exchange factor.

As described above, C. albicans homologues of Saccharomyces cerevisiae CDC42, BUD1, and BEM1 genes were also by suppression of a Saccharomyces cerevisiae cdc24 temperature sensitive mutant. CaCdc42 protein is 88% identical to its budding yeast counterpart.

B10 Functional analysis in S. cerevisiae

We first investigated whether the C. albicans Cdc24p could fuinctiona in S. cerevisiae. Two assays were used: a growth complementation test in cdc24-ts mutants and a mating assay in a cdc24-m1 mutant. As disclosed above, the latter mutant is specifically deficient in orientation of the mating projection towards a pheromone gradient resulting in a mating defect. In both cases C. albicans Cdc24p was expressed on a muilti-copy plasmid from a strong promoter (TPI) and amino terminally HA-epitope tagged. For growth assays 3 diffcrent cdc24-ts mutants were grown at a range of temperatures with and without 1 M sorbitol. Sorbitol has been shown to reduce the severity of growth defects of cdc24-ts alleles at non-permissive temperatures (Bender and Pringle, 1989). C. albicans Cdc24p did not rescue the lethality of any cdc24-ts mutants at non-permissive temperatures (data not shown). Slight toxicity was evident in all three ts mutants compared to an empty vector and S. cerevisiae CDC24 controls grown at 30° C. both with and without 1 M sorbitol (data not shown). Immunoblots probed with anti-HA antisera revealed a correctly sized protein in these cells at levels similar to S. cerevisiae Cdc24p (data not shown). C. albicans Cdc24p over-expression in a cdc24-mI mutant slightly suppressed the mnating defect, with a two fold increase in mating efficiency (Table IV).

Table IV
C. albicans CDC24 increases mating efficiency of a
cdc24-ml mutant by 2 fold.

| | No: of diploids when mated with wildtype |
|---|---|
| RAY1044 containing | mating partner* |
| p2ATPIHA | 34.5 (+/−0.5) |
| p2ATPIHAScCDC24 | >250 |
| p2ATPIHACaCDC24 | 71 (+/−6.0) |

*Figures arc means of two independent determinations. Errors are maxima and minima of these determinations.

Therefore, C. albicans CDC24 appears non-functional in S. cerevisiae. A possible explanation for this is either that C. albicans CDC24 is toxic when expressed in S. cerevisiae or the CTG codon reassignment (Santos et al, 1993) compromises the flmction of C. albicans Cdc24p in S. cerevisiae. C. albicans CDC24 contains 4 CTG codons that result in leucines when expressed in S. cerevisiae rather tian serines in C. albicams. One of these (position 648) is conserved in Cdc24p sequences from S. cerevisiae, K. lactis, C. albicans , and S. pombe.

B11 CDC24 is essential for viability in C. albicans

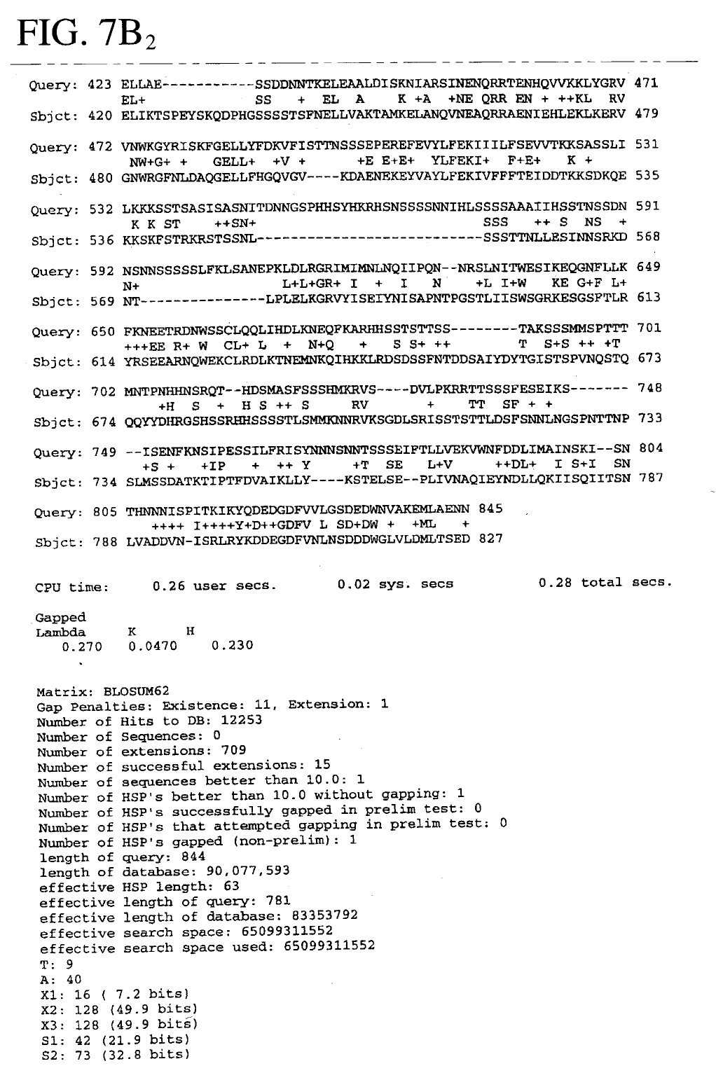

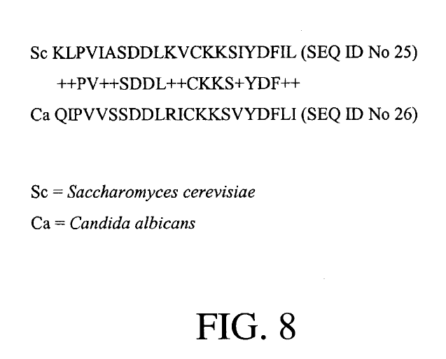

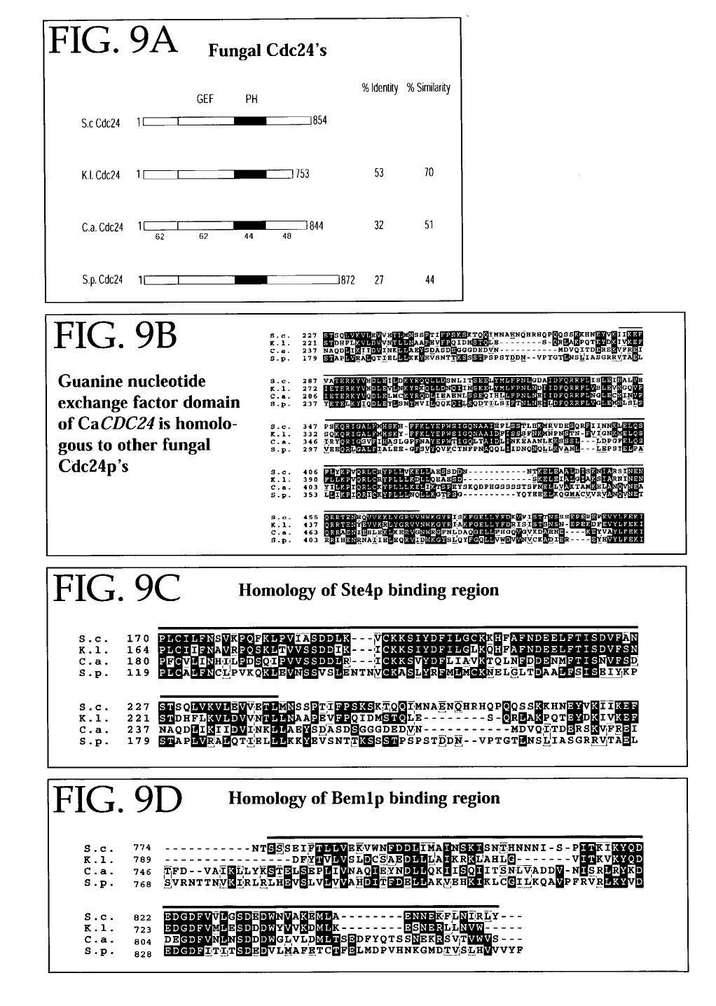

While in S. cerevisiae CDC24 is essential, in S. pombe this gene (SCD1) is not required for viability. Therefore we examined the requirement of CDC24 in C. albicans. To assess the fimction of C. albicans Cdc24p we constructed a strain in which expression of a single copy of CDC24 was driven by a regulated promoter. The best characterised regulated promoter in C. albicans is the S. cerevisiae MET3 promoter (MET3P) homologue. Expression from this promoter in both S. cerevisiae and C. albicans is completely represscd by metionine and cysteine at mM levels (Care, R. S. et al, 1999 Leng, A. et al, 2000). A C. albicans CDC24/cdc24Δ (PY12) was inade by targetted gene replacement using a cdc24Δ::HIS1 cassette. This cassette replaces all but the carboxy-terminal 21 amino acids of CDC24 with the HIS1 gene (FIG. 10A). Knock-out transformants were confirmed by PCR (FIG. 10B) and were then used to make a mutant in which the MET3P was integrated 3′ of the sole CDC24 copy (PY18) (FIG. 10A). Correct integration of the MET3P was confirmed by PCR (FIG. 10C) and sequencing of the PCR product from the MET3P to CDC24.

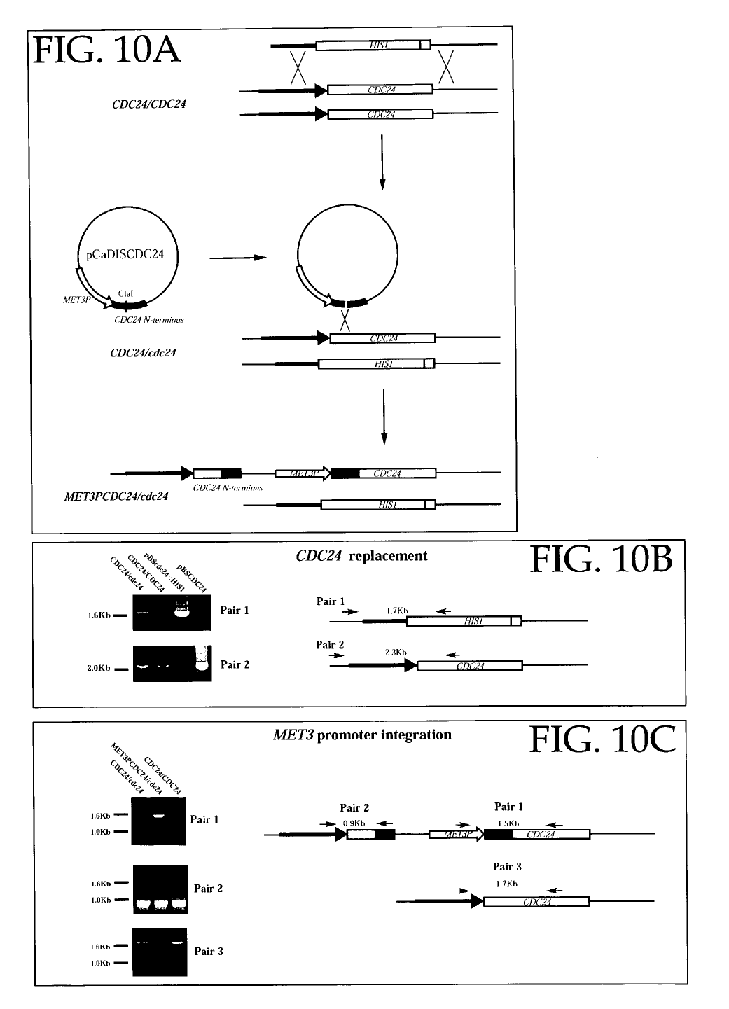

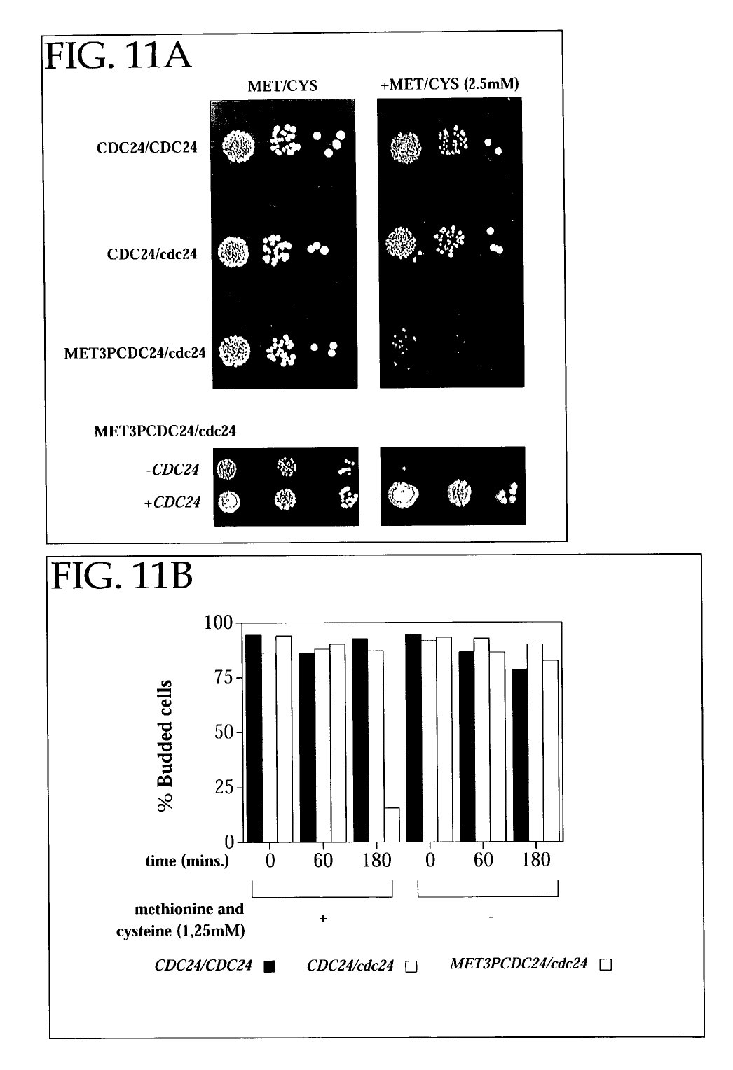

To examine the function of Cdc24p cells were grown in the presence and absence of met and cys. While the wild-type and heterozygote stains grew normally on SC plates containing met and cys the MET3PCDC24/cdc24Δ (PY18) strain was inviable (FIG. 11A). At the highest cell concentrations some growth was observed in the MET3PCDC24/cdc24Δ strain which was attributed to spontaneous revertion. Reintroduction of CaCDC24, integrated at the RP10 ribosomal subunit locus, into CaMet3ₚᵣₐₘCaCDC24/Δcacdc24 strains restored growth in Met and Cys media (FIG. 11A).

To oimer characterise this strain, cells were examined after 1 and 3 hr in liquid media containing or lacking met and cys. Strikingly, MET3PCDC24/cdc24Δ cells arrested as round unbudded cells in the presence of met and cys (FIG. 11B). After 3 hr repression, cell numbers for the MET3PCDC24/cdc24Δ strain were approximately half that of the other two strains. This grow arrest was reversible as the colony forming units were equal to cell counts from the liquid cultures. In the absence of met and cys wildtype, CDC24/cdc24Δ and MET3PCDC24/cdc24Δ cells all showed the characteristic unipolar (budding at a single cell pole) budding pattern (Yaar, L. et al, 1997) assayed using Calcoflinor white and subsequent visualisation of bud scars (data not shown). These results indicate that CDC24 is an essential gene in C. albicans.

Strains with regulated expression of CaCDC42, CaBUD1 and CaBEM1 were examnined simnilarily in order to determine if the observed growth defect was specific for Cacdc24 (FIG. 11C). While all strains grew similarly on media lacking Met and Cys, specifically Cacdc42 cells were inviable in the presence of Met and Cys. Even after prolonged incubation no colonies were observed when Cacdc42 cells were spotted on Met and Cys containing media. In contrast the Cabud1 strain grew normally on Met and Cys media. Lastly, in the presence of Met and Cys, Cabem1 cells grew poorly, exhibiting similar growth defects as Cacdc24 strains.

The G-protein CaCdc42 and its exchange factor CaCdc24 are thus necessary for normal C. albicans growth.

B12 C. albicans CDC24 is required for hyphal growth

Candida albicans becomes hyperpolarised in response to serum (Barlow et al, 1974), body temperature (37° C.) or neutral pH. In these conditions C. albicans switches from the budding (vegetative) yeast form to an elongated hyphal form that is capable of invading solid surfaces. To elucidate their role in invasive hyphal formation, we examined the various strains for their ability to invade a solid agar surface. We initially screened for conditions in which MET3PCDC24/cdc24Δ cells grew similarly to wild-type. Growth was examined at 30° C. and 37° C. both on YEPD and on SC media containing increasing concentrations of met. Two optimal growth conditions were YEPD and SC lacking met and cys. Metionine concentration in YEPD determined by amino acid composition analysis was 1.0 mM. This concentration of met in synthetic media was sufficient to significantly repress growth of MET3PCDC24/cdc24Δ cells. To determine if both met and cys were necessary for MET3P repression met or met and cys were added to YEPD plates. Addition of 0.5 mM met and cys was sufficient to markedly repress growth on YEPD, whereas addition of only 0.5 mM met had little effect (data not shown).

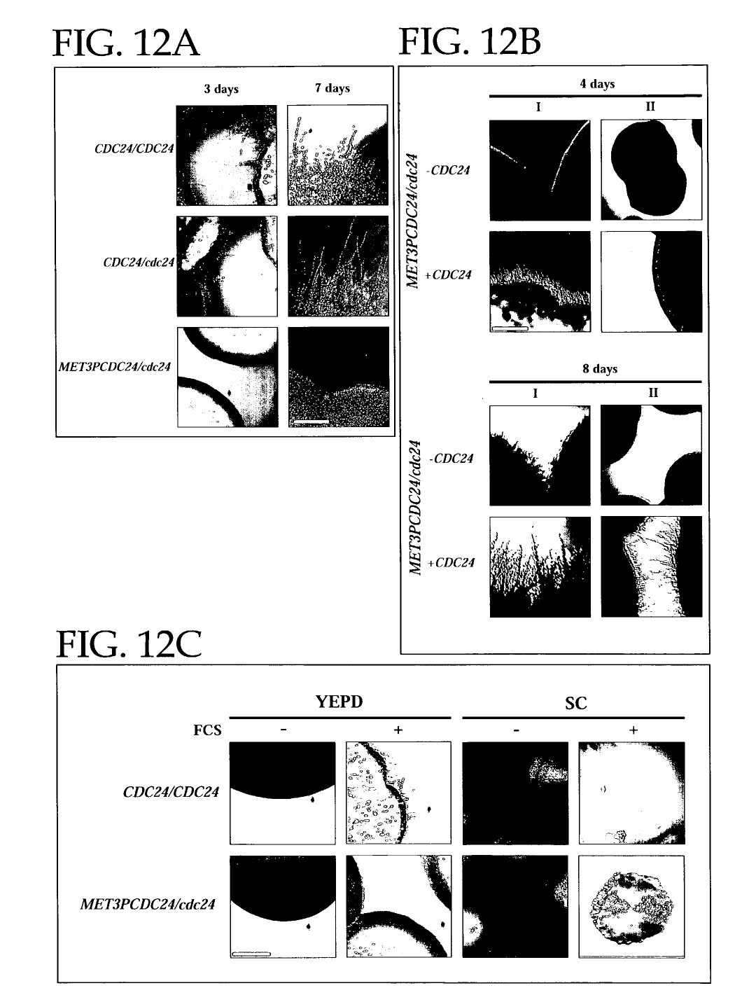

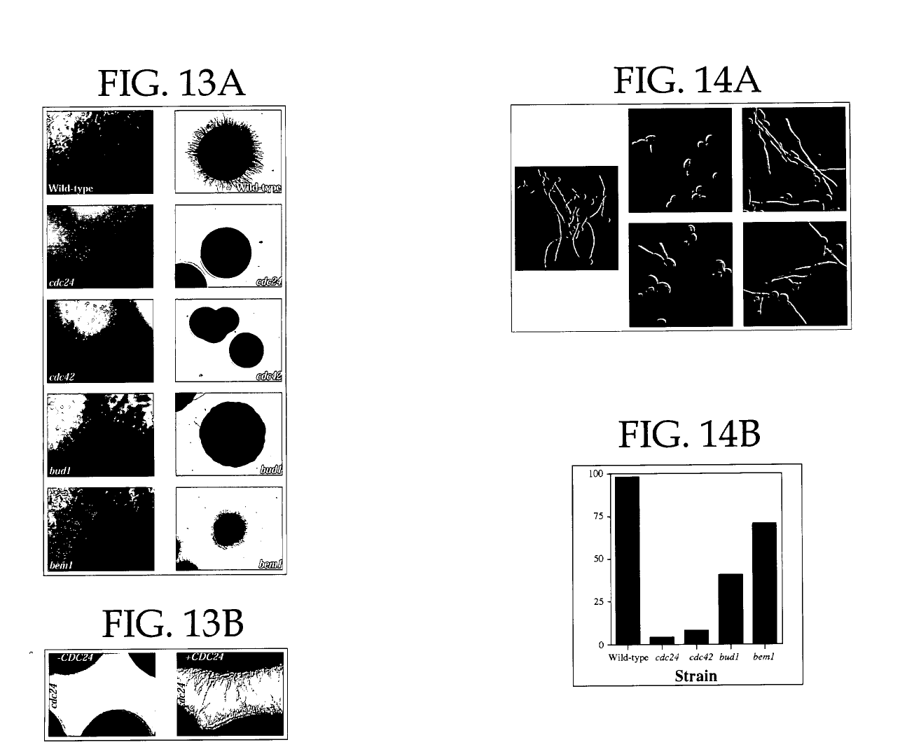

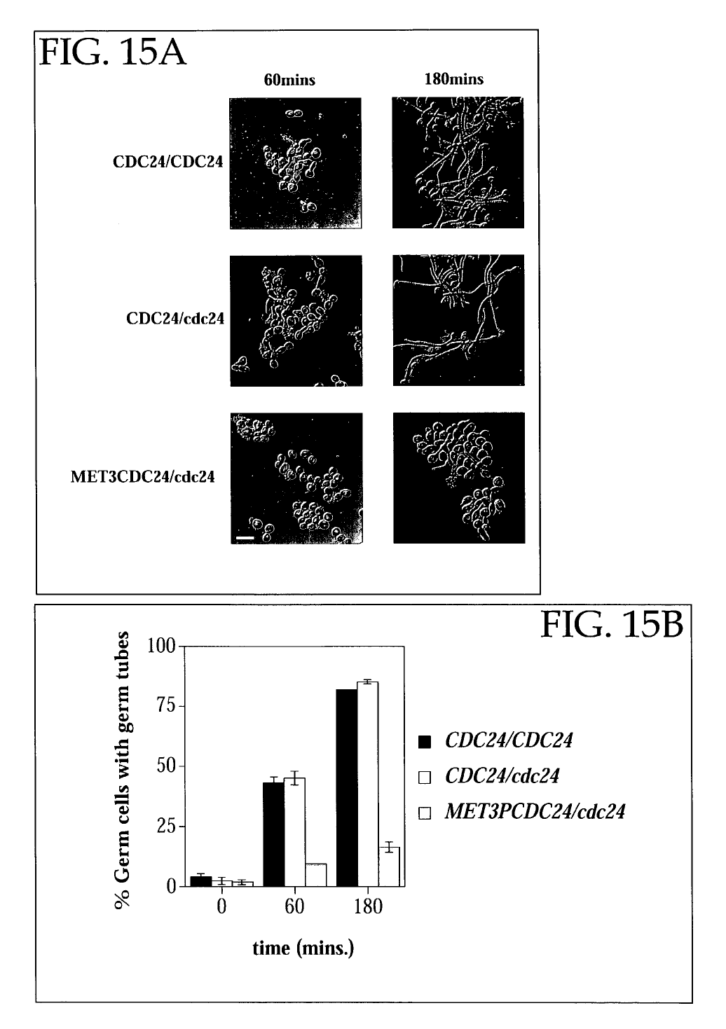

Hyphal formation was assessed using two methods: growth on agar plates and examination of cell morphology in liquid media. Invasive hyphal growth was determined by spotting equal amounts of cells on YEPD containing DFCS. FIG. 12A shows that both wild-type and CDC24/cdc24Δ cells invade agar after 3 days, with an increase in invasion and number of hyphae after 7 days. In contrast, the MET3PCDC24/cdc24Δ strain was severely defective in hyphal formation and invasion. Even after 13 days MET3PCDC24/cdc24Δ cells showed only very slight invasion. Addition of a genomic copy of CDC24 completely rescued this defect in invasive hyphal growth (FIG. 12B). This result indicates that the defect is recessive and not a dominant negative effect of Cdc24p overexpression.

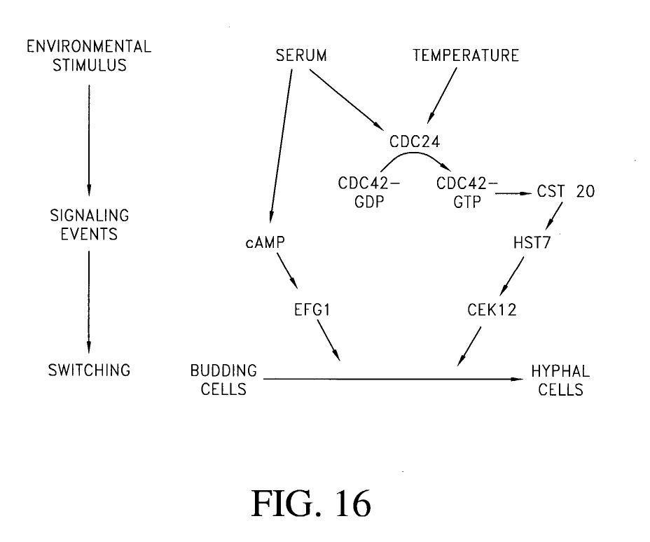

Similarly, Cacdc42 strains, while able to grow normally, did not become hyphal or invade the solid surface (FIG. 13A). Even after prolonged incubation (greater than 10 days) colonies of this strain continued to grow but did not reveal any invasive hyphal growth. In contrast, Cabem1 colonies grew in an invasive hyphal fashion more or less normally in similar conditions. Colonies of Cabud1 cells were intermediate between these two extremes (Cacdc24/Cacdc42 and Cabem1) and became invasive yet slower than the wild-type cells (FIG. 13A).

To furter characterise these defects, cells were grown on different media at both 30° C. or 37° C. FIG. 12C shows there is no difference in wild-type invasive growth on YEPD containing either FCS or DFCS (c.f. FIG. 12A) whereas no invasive growth was observed on media lacking serum. However, after 7 days at both 30 and 37° C. wild-type and heterozygote strains invaded YEPD and SC plates lacking DFCS whereas MET3PCDC24/cdc24Δ cells and Cacdc42 cells did not. On SC−met−cys containing DFCS wild-pe cells were able to invade agar but invasion morphology was different with extensively branched hyphae. In general colony size on SC were smaller than those on YEPD and colonies were not crenilated. Taken together these data suggest that the invasive growth defect of MET3PCDC24/cdc24Δ is independent of growth media indicating that CDC24 and CDC42 are required for invasive growth under all conditions we tested.

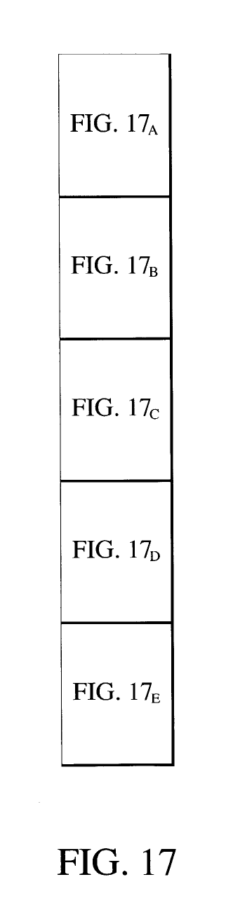

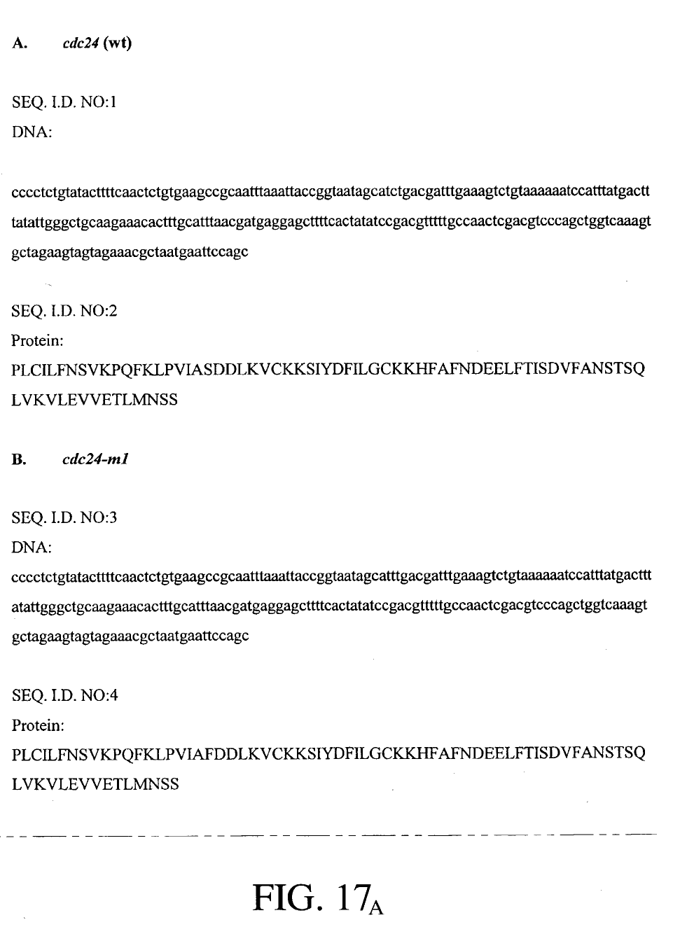

A defect in invasive byphal growth could be dae to either an inability of yeast-form cells to become hyper-polarized and form germ tubes, i.e. initiate hyphal growth, or to an inabilty to maintain macroscopic filamentous hypbae. To distinguish between these possibilities we analyzed the behaviour of the above described strains in liquid YEPD media containing FCS at 37 C. These conditions resulted in approximately 50% and greater htan 90% of wild-type cells with germ tubes after one and three hours, respectively. After 3 hours wild-type cells diplayed elongated germ tubes, with each many times the lengthl of the cell body (FIG. 14A). Qualitatively Cabud1 and Cabem1 strains appeared similar to wild-type cells. Determination of the percentage of cells with germ tubes showed that the Cabud1 strain had an approximately two-fold reduction compared to wild-type cells, whereas the Cabem1 strain showed about 70% germ tubes relative to wild-type strains. Strikingly Cacdc24 and Cacdc42 cells appeared to have little to no germ tubes (FIG. 14A) with a quantitation revealing a 20-fold and 12-fold reduction in the number of cells with germ tubes (FIG. 14B). The Cacdc24 cells nonetheless grew in the presence of FCS as indicated by the presence of budded cells. Closer examination revealed that a portion of dle buds appeared elongated, reminiscent of germ tube initiation. This effect was more pronounced with the Cacdc42 strain where cells were evident with elongated buds or daughter cells which were rougluy twice the length of the nother.

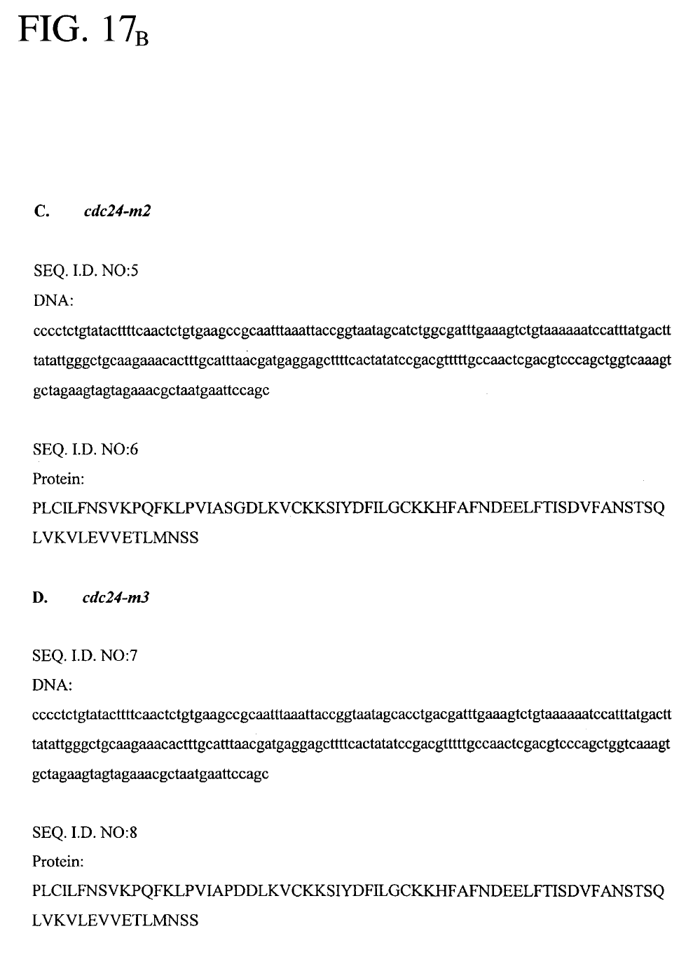

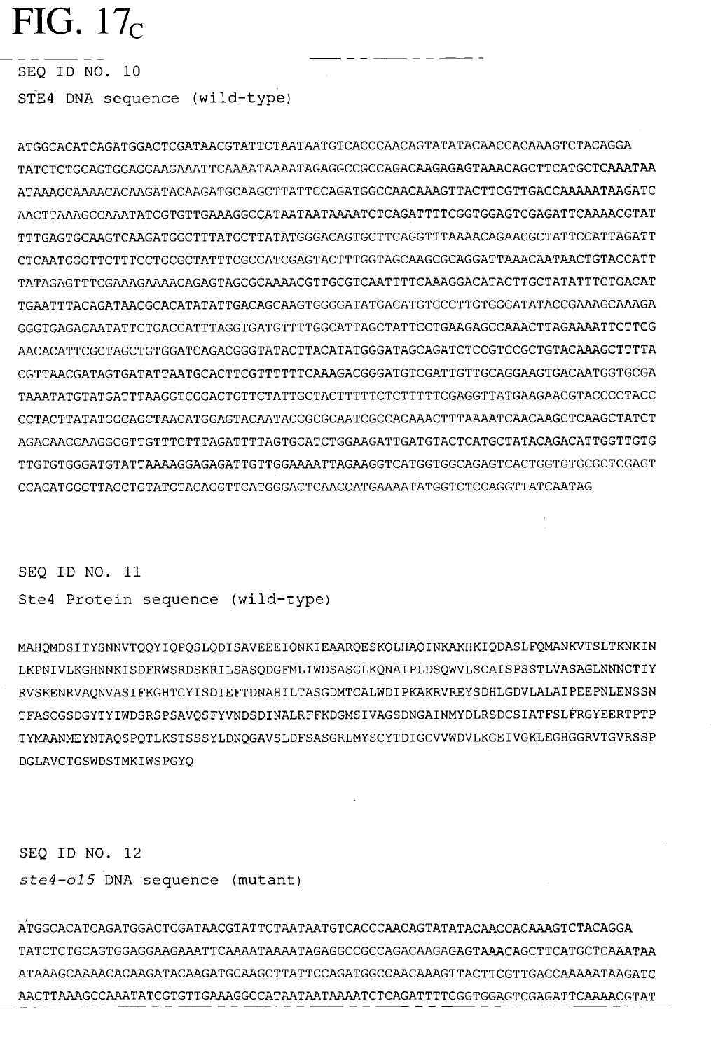

Identical results were obtained when strains were treated similarly at 30° C. or when they were incubated in synthetic complete media lacking Met and Cys containing dialyzed FCS. We exarnined cells after incubation at 37° C. in SC—met—cys with DFCS. FIG. 15A shows differential interference contrast (DIC) images of wild-type, CDC24/cdc24Δ and MET3PCDC24/cdc24Δ cells grown for 3 hr at 37° C. in liquid media. Both the wildtype and CDC24/cdc24Δ cells responded to serum after 60 min. with approximately 45% of cells having germ tubes. After 3 hr the number of cells with germ tubes increased to 85%. Stikingly, MET3PCDC24/cdc24Δ cells were severely defective in germ tube formation. Even after 3 hr in DFCS at 37° C. they hardly formed germ tubes with a 4-5 fold decrease in the number of cells with genn tubes compared to wild-type and CDC24lcdc24Δ cells (FIG. 15B). Visual inspection of these cultures suggest that MET3PCDC24cdc24Δ cells continued to bud whereas wild-type and CDC24lcdc24Δ cells arrested vegetative growth and formed germ tubes in the presence of DFCS. Thus, there is a specific requirement for CDC24 in hyphal growth or switching to hyphal growth which is defective in MET3PCDC24/cdc24Δ cells.

Thus, repression of CDC24 in MET3PCDC24/cdc24Δ cells by met and cys results in an inability to bud and constitutive expression of CDC24 in the presence of met and cys results in severe defects in both germ tube formation and invasive growth in response to serum and elevated temperature. These results show that under certain conditions MET3PCDC24/cdc24Δ cells are able to grow but are unable to form germ tubes and invade agar suggesting a specific function of CDC24 in the hyphal switch or in maintaining hyphal growth. In summary, the above results show that C. albicans CDC24 and CDC42 are each required both for bud and hyphal formation in both liquid and solid media.

B13 CDC24 and CDC42 are Required for C. albicans Virulence

Our results show that CaCdc24 and CaCdc42 are required for invasive hyphal growth in both liquid and solid media. To determine whether these proteins were necessary for Candida albicans virulence we innoculated mice with the different strains and analyzed lethality and kidney colonization. A URA3+ wild-type strain was used as a control in order to circumvent the reduced virulence of ura3+ strains. Mice were injected intravenously with 1/33 10⁶ cells of wild-type, Cacdc24, Cacdc42, Cabud1, or Cabeml C. albicans strains. The wild-type strain resulted in 50% mortality after 5 days, whereas even after 40 days Cacdc24 and Cacdc42 strains had no effect on mouse mortality. The Cabudl strain exhibited reduced mouse mortality compared to the wild-type C. albicans with 50% mortality observed after 9 days. Cabeml cells, while similar to Cacdc24 cells with respect to reduced growth on Met and Cys containing media (repessive conditions), nonetheless resulted in 30% mice mortality after 16 days. The kidneys from two mice were removed after 1 and 3 days post injection and the number of colony forming units was anlayzed. One day post innoculation CFUs per kidneys from mice injected with Cacdc42 or Cabeml C. albicans were 17-fold reduced compared to wild-type controls. Three days post innoculation, the CFU of kidneys from mice injected with these two strains was 30-40-fold reduced compared to wild-type controls. Additional mice experiments were carried out with 10-times the initial intravenous dosage. With these high infection levels, Cacdc42 cells were substantially reduced in mice mortality with no deaths observed until 9 days post-injection (40% mortality after 12 days), a time in which all mice injected with the other strains were dead. While these innoculation levels resulted in mortality of mice injected with Cacdc24 cells, mortality induced by this C. albicans strain was monetheless reduced when compared to mice injected with wild-type, Cabudl, or Cabeml yeast. In these conditions, two days post-innoculation CFUs from mice kidneys injected with different C. albicans strains were similar except Cacdc24 injected mice, which had a 40-fold reduction in the kidney CFUs. In all conditions the genotype of the yeast colonies recovered from the sacrificed mouse kidneys was identical to the starting strains, indicating that no substantial gene recombination or rearrangement had occurred. Together our results demonstrate that Cacdc24 and Cacdc42 strains are substantially reduced in pathogenicity using the intravenous mouse model, suggesting that these two proteins are necessary for virulence.

B14 DISCUSSION Section B

Cdc24p belongs to a diverse family of GEFs which include many mammalian proto-oncogenes². This group of proteins shares a conserved region consisting of a Dbl-domain (named after the human proto-oncogene Dbl) followed by a plecktstrin-homology domain (PH).

We have sequenced the entire CDC24 gene including promoter and terator regions from C. albicans. As described above, sequence comparison between a Cdc24p obtainable from S. cerevisiae and C. albicans show about 32% identity and 51% similarity using a conventional BLAST line up. In particular, a comparison between the critical regions in the Cdc24p obtainable from S. cerevisiae (as identified above) and the corresponding region in the Cdc24p obtainable from C. albicans indicated that of 22 amino acids, 13 were identical (59% identity) and 7 were similar (32%). The 19 amino acid fragment of C. albicans corresponding to the 19 amino acid piece of the S. cerevisiae Cdc24p with similarity to the human proto-oncogene Dbl shares 89.5% homology with the S. cerevisiae Cdc24p 19 amino acid fragment. The 76 amino acid fragment of C. albicans corresponding to amino acids 170 and 245 in S. cerevisiae shares 75.0% homology with the corresponding S. cerevisiae fragment.

Furternore, we have shown that the Cdc24p obtainable from C. albicans provides a similar connection between G-protein coupled receptor activation and polarsed cell growth as the Cdc24p from S. cerevisiae. We have examined the funIction of C. albicans CDC24 in mitotic growth (budding), in hyphal formation and invasive growth. Our results indicate that CDC24 is required for viability since a mutant in which the MET3 promoter regulates a sole copy of CDC24 is inviable when grown in conditions that repress the MET3 promotcr. Under repressive conditions these cells arrest growth as unbudded cells. Following exposure to serum and/or 37° C C. albicans cells become hyperpolatised, form germ tubes, extended hyphae, and invade solid surfaces. Constitutive expression of CDC24 from the MET3 promoter results in a severe defect in invasive growth due to the inability to form germ tubes, in contrast bud formation appears normal. These results suggest a specific function of Cde24p in hyphal formation or mnaintainence of the hyphal state.

Our findings are consistent with results from other fungi. In S. cerevisiae cdc24 temperature sensitive mutants arrest as large spherical unbudded cells with delocalised deposition of cell wall chitin (Sloat et at, 1978, 1981). It is likely that the defect of this mutant in restricting secretion is due the observed delocalisation of the actin cytoskeleton (Sloat et al,1981, Sloat et al, 1978, Hartwell et al, 1973). As disclosed herein, Cdc24p is also required during haploid cell mating for orientation of the mating projection towards the pheromone gradient of a mating partner. Instead of orienting their mating projection towards a pheromone gradient, cdc24-m1 mutants form a mating projection adjacent to the previous bud site. In this mutant lhe actin cytoskeleton polarises correctly and secretion is properly localised to the tip of the mating projection. In Schizosaccharomyces pombe the CDC24 homologue SCD1 is not essential for viability. However, Δscd1 cells are round, in contrast to their normal elongated shape, and defective in mating (Chang et al, 1994). S. pombe CDC42 is also involved in polarised growth as overexpression of dominant lethal forms leads to aberrant cell morphologies (Miller et al, 1994). In the pathogenic lungus Wangiella dermatitidi CDC42 is not essential however overexpression of constitutively active cdc42 suppresses hyphal formation and invasion (Ye and Szaniszlo, 2000). Our results suggest the function of the Cdc24p/Cdc42p exchange factor/GTPase module is conserved and has a key role in polarised growth.

Many studies have shown that a number of different genes are required for C. albicans to switch between yeast and hyphal forms. These genes comprise two morphogenetic signalling pathways in C. albicans. One of these pathways is homologous to the S. cerevisiae mating pheromone response MAP kinase pathway and the otler a honmologuc of the cAMP/protein kinase A mediated pathway. C. albicans hoinologues of STE20 (CST20), STE7 (HST7) and STE12 (CEK12) are necessary for hyphal growth (Lui et al 1994; Leberer et al 1996; Kohler and Fink, 1996; Clark et al, 1995).

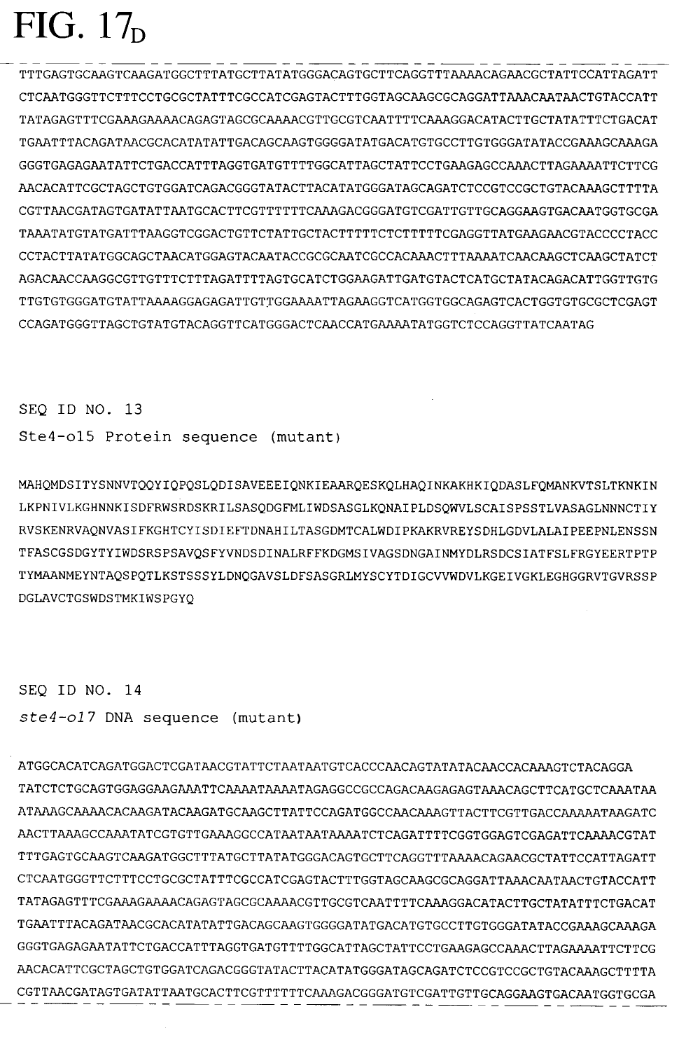

In S. cerevisiae Cdc42p signaling via Ste20p is necessary for filamentous growth in diploids (Mosch et al, 1996; Lui, et al, 1993). Hence we speculate that in C. albicans CDC24 may regulate hyphal formation and invasion by signaling through CDC42 to activate the STE20/initogen-activated signalling pathway (FIG. 16). One prediction of this model would be that constitutively active Cdc42p or kinases (Ste20 and MAP kinases) might rescue the hyphal defect described in this work. It is possible that signalling via the cAMP pathway required for hyphal growth (Stoldt et al, 1997) may explain why after extended times the MET3 promoter regulated cdc24 mutant is able to invade agar and forms small hyphae in liquid media. This is consistent with the observation that mutants in the C. albicans mating MAP kinase pathway still respond to serum in liquid media (Lo et al, 1997).

Our work demonstrates that CDC24 in C. albicans has two functions; an essential function in bud formation and a non-essential function in hyphae formation. This is directly analogous to its fiunctions in S. cerevisae in budding and mating. Constitutive expression of C. albicans Cdc24p results in a hyphal defective although has no effect on budding; implying that some regulation of Cdc24p is required for the dimorphic switch. This hypothesis is supported by the observation that the rates of increase in the level of the C. albicans CDC42 transcript vary between budding and hyphal formation. CDC42 mRNA levels increase rapidly during budding and only slowly during hyphal formation (Mirbod et al, 1997). It is unlikely that the hyphal defect of MET3 promoter regulated CDC24 cells is due to an overexpression of Cdc24p as this defect is recessive to wild-type copies of CDC24. Rather, we imagine that a change in the level of Cdc24p might be required to initiate the hyphal switch and this cannot occur in the MET3 promoter CDC24 strain.

To determine if the defect on germ tube formation was a kinetic defect, i.e. due to a slowing of the morphological transition, we detennined the percentage germ tubes after 1 and 3 hours in YEPD FCS. FIG. 15B shows that the percentage of Cacdc24 cells with germ tubes was sinilar at both times, suggesting that the defect is not due to slower morphological transition. Furthermore, prolonged incubation of Cacdc24 cells in YEPD FCS revealed similar defects. In addition, the observed defects were only seen with CaMet3ₚᵣₒₘCaCDC24/Δcacdc24 (Cacdc24) cells and not with CaCDC24/Δcacdc24 heterozygotes or CaMet3ₚᵣₒₘCaCDC24/Δcacdc24 cells in which an additiona copy of CaCDC24 was reintroduced. The behavior of these strains in liquid media containing serum is consistent with the notion that CaCdc24 and CaCdc42 are required for the yeast—hyphal morphological transition. Furthermore our results suggest that Cacdc24 and Cacdc42 strains can bitiate cell polarization which precedes hyphal formation, yet are unable to maintain this directional or unipolar growth. Furtherrnore, we have shown that CDC24 and CDC42 are required for Candida albicans virulence.

In summary, CDC24 in Candida albicans is essential for viability and in addition constitutive expression from the MET3 promoter resuilts in a defect in hyphal formation. We propose that a change in the level of the Cdc24p/Cdc42p exchange factor/GTPase module is required for switching between yeast and invasive forms of this fuangal pathogen.

### SUMMARY

1) We have identified an important interaction between two general cellular components, Cdc24p and Gβ which provides a connection between G protein coupled receptor activation and polarised cell growth. This work has been exemplified by work done with yeast genes/proteins, however, both cellular components involved have homologues in humans.

2) We show the physiological consequence of lis interaction and from these data extrapolate to the general role of this interaction in human cells.

3) In addition, we have identified sequences required for this interaction. Specifically, we have identified a short stretch of one protein (Cdc24p) encompassing 76 aa sufficient for this interaction and three amino acid changes (within this stretch) which block the interaction and have physiological consequences. These amino acid changes fill within a 19 amino acid piece with similarity to the human protooncogene Dbl. Indeed, removal of this region from proto-Dbl (when the amino terminus is removed) results in oncogenicity in tissue culture cells.

4) We have also identified specific mutants in the β-subunit of the heterodimeric G protein (Ste4p) which appear to block its interaction wieh Cdc24p. We believe that several of these mutations will fall in conserved regions of Gβ. Thus, it is possible to devise assays based on this mutation to screen for agents capable of modifying the non-interactive behaviour of the mutant G protein β subunit with Cdc24p. In addition, the assay could be used to study Cdc24p homologues or even Cdc24p derivatives or homologues to see if those derivatives or homologues affect the non-interactive behaviour of the mutant G protein.

5) There is a wealth of information on human Gβ's, human GEF's (GDP/GTP Exchange Factors), such as Cdc24p homologues and the rho family of GTP-binding-proteins (such as rho like Cdc42p) which the GEFs work on. Most human GEF's are oncogenes such as DbI, Vav, and Ect and are involved in some way in growth control. Furthermore Gβ's are involved in linking signals from receptors to intracellular responses. The present invention has shown that that a GEF from yeast, Cdc24p, can directly bind Gβ in the absence of any other yeast proteins. Although unproven, it is likely that interactions between human GEF's and Gβ's are also crucial in growth control and chemotaxis.

6) We propose the interaction we have identified will have broad cellular ramifications and manipulation of these interactions (such as peptidic inhibitors and peptides mimicking activated species) will be of therapeutic value.

7) In addition, simple yeast based assays systems could be extremely useful for high through-put screening to identify mnolecules perturbing this interaction. In particular, a qualitative assay Lising a yeast mutant with a mating defect could prove useful in the design of agents, such as anti-cancer agents, that can affect the function of oncogenes such as proto-Dbl, in terms of its ability to complement a yeast mutant mating defect and/or its function in mamnalian tissue culture cells.

8) We also believe similar interactions will be ideal targets for anti-fgal drugs directed at invasive and pathogenic yeasts such as Candida albicans and Cryptococcus neoformans.

9) Accordingly, we have sequenced the entire CDC24 gene including promoter and terninator regions obtainable fromn C. albicans. The C. albicans Cdc24p is a protein essential for viability and the life and growth of yeasts such as those obtainable from Candida species such as C. albicans. A sequence comparison between the Cdc24p obtainable from S. cerevisiae and C. albicans show about 32% identity and 51% similarity using a conventional BLAST line up.

10) We have already identified an important interaction between two general cellular components, Cdc24p and Gβ which provides a connection between G protein coupled receptor activation and polarised cell growth (see earlier). This work has been exemplified by work done with yeast genes/proteins, however, both cellular components involved have homologucs in humans. The Cdc24p obtainable from C. albicans may provide an appropriate target for inhibition of cell growth.

11) A sequence comparison between the sequences which are required for the interaction between two general cellular components, Cdc24p and Gβ, in the Cdc24p obtainable from S. cerevisiae (as outlined above) arnd the corresponding region in the C. albicans Cdc24p indicated that of 22 amino acids, 13 were identical (59% identity) and 7 were similiar (32%). Significantly, we have shown that the 19 amino acid fragment of C. albicans corresponding to the 19 amino acid fragment of the S. cerevisiae Cdc24p with similarity to the human protooncogene Dbl shares 89.5% homology with the S. cerevisiae Cdc24p 19 amino acid fament and the 76 amino acid fragment of C. albicans corresponding to amino acids 170 and 245 in S. cerevisiae shares 75.0% homology with the corresponding S. cerevisiae fragment.

12) We have shown that C. albicans Cdc24p interactions have broad cellular ramifications and mnanipulation of these interactions (such as peptidic inhibitors and peptides mimicking activated species) may be of therapeutic value in anti-fungal treatments.

13) l addition, simple yeast based assays systems could be extremely useful for high through-put screening to identify molecules perturbing this interaction. In particular, a qualitative assay using a yeast mutant with a mating defect may prove useful in the design of agents, such as anti-fingal agents.

14) We have shown that Cdc24p and Cdc42p are essential for viability in C. albicans.

15) We have shown that a fnctioning CD C24 gene is required for hyphal growth in C. albicans. Similarly, we have also shown that a flnctioning CDC42 gene is required for hyphal growth in C. albicans.

16) Thus we have shown that both Cdc24p and Cdc42p are required for budding and hyphal formation in C. albicans.

17) We have also shown that Cdc24p and Cdc42p are required for virulence of C. albicans in mice.

18) Thus C. albicans Cdc24p GEF interactions inay be an ideal target for anti-fiudgal drugs directed at invasive and pathogenic ycasts such as Candida albicans and Cryptococcus neoformans and Aspergillus niger. Moreover Cdc42p may similarly be a target.

All publications mentioned in the above specification are herein incorporated by reference. Various modifications and variations of the described methods and system of the invention will be apparent to those skilled in the art without deparrrg from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### REFERENCES

1. Machesly, L. M. & Hall, A. Rho: a connection between membrane receptor signaling and the cytoskeleton. Trends In Cell Biology 6, 304-310 (1996).

2. Cerione, R. A. & Zheng, Y. The Dbl family of oncogenes. Current Opinion In Cell Biology 8 216-222 (1996).

3. Whiteway, M., et al. The STE4 and STE18 genes of yeast encode potential b-subunits and γ-subunits of the mating factor receptor-coupled G protein. Cell 6, 467-477 (1989).

4. Sprague, G. F. J. & Thorner, J. W. Pheromone response and signal transduction during the mating process of Saccharomyces cerevisiae. In The molecular and cellular biology of the yeast Saccharomyces (eds. Jones, E. W., Pringle, J. R. & Broach, J. R.) 657-744 (Cold Spring Harbor Press, Cold Spring Harbor, N.Y., 1992).

5. Leberer, E., Thomas, D. Y. & Whiteway, M. Pheromone signalling and polarized morphogenesis in yeast. Current Opinion in Genetics & Development 7, 59-66 (1997).

6. Simion, M. -N, et al. Role for the Rho-family GTPase Cdc42 in yeast mating pheromone signal pathway. Nature 376, 702-705 (1995).

7. Zhao, Z. S., Leung, T., Manser, E. & Lim, L. Pheromone signalling in Saccharomyces cerevisiae requires the smrall GTP-binding protein Cdc42p and its activator CDC24. Mol Cell Biol 15, 5246-57 (1995).

8. Sloat, B. F., Adams, A. & Pringle, J. R. Roles of the CDC24 gene product in cellular morphogenesis durig the Saccharomyces cerevisrae cell cycle. J Cell Biol 89, 395-405 (1981).

9. Adams, A. E., Johnson, D. I., Longnecker, R. M., Sloat, B. F. & Pringle, J. R. CDC42 and CDC43, two additional genes involved in budding and the establishment of cell polarity in the yeast Saccharomyces cerevisiae. J Cell Biol 111, 131-42 (1990).

10. Chenevert, J., Valtz, N. & Herskowitz, I. Identification of genes required for normal pheromone-induced cell polaiization in Saccharomyces cerevisiae. Genetics 136, 1287-96 (1994).

11. Trueheart, J., Boeke, J. D. & Fink, G. R. Two genes required for cell fuision during yeast conjugation: evidence for a pheromone-induced surface protein. Mol Cell Biol 7, 2316-2328 (1987).

12. Segall, J. E. Polarization of yeast cells in spatial gradients of a mating factor. Proc Natl Acad Sci USA 90 8332-6 (1993).

13. Dorer, R,. Pryciak, P. M. & Hartwell, L. H. Saccharomyces cerevisiae cells execute a default pathway to select a mate in the absence of pheromone gadients. J Cell Biol 131, 845-61(1995).

14. Valtz, N, Peter, M. & Herskowitz, I. FAR1 is required for oriented polarization of yeast cells in response to mating pheromones. J Cell Biol 131, 863-73 (1995).

15. Zheng, Y., Cerione, R. & Bender, A. Control of the yeast bud-site assembly GTPase Cdc42. Catalysis of guanine nucleotide exchange by Cdc24 and stimulation of GTPase activity by Bem3. J Biol Chem 269, 2369-72 (1994).

16. Zheng, Y., Bender, A. & Cerione, R. A. Interactions among proteins involved in bud-site selection and bud-site assembly in Saccharomyces cerevisiae. J Biol Chem 270 626-30 (1995).

17. Chenevert, J., Corrado, K., Bender, A., Pringle, J. & Herskowitz, I. A yeast gene (BEM1) necessary for cell polarization whose product contains two SH3 domains. Nature 356, 77-9 (1992).

18. Hirschman, J. E., DeZutter, G. S., Simonds, W. F. & Jenness, D. D. The Gβγ complex of the yeast pheromone response pathway—subcellular fractionation and protein-protein interactions. J Biol Chem 272, 240-248 (1997).

19, Peter, M., Neiman, A. M., Park, H. O., Van Lohuizen, M. & Herskowitz, I. Functional analysis of the interaction between the small GTP binding protein Cdc42 and the Ste20 protein kinase in yeast. Embo Journal 15, 7046-7059 (1996).

20. Leberer, E., et al. Functional characterizaton of the Cdc42p binding domain of yeast Ste20p protein kinase. Embo Journal 16, 83-97 (1997).

21. Rose, M. D., Winston, F. & Hieter, P. Methods in yeast genetics: a laboratory course manual (Cold Spring Harbor Press, Cold Spring Harbor, N.Y., 1991).

22. Miyanioto, S., et al. A DBL-homologous region of the yeast CLS4C/DC24 gene product is important for Ca(2+)-modulated bud assembly. Biochem Biophys Res Commun 181, 604-10 (1991).

23. Mitchell, D. A., Marshall, T. K & Deschenes, R. J. Vectors for the inducible overexpression of glutathione-S-transferase faision proteins in yeast. Yeast 9, 715-722 (1993).

24. Wach, A., Brachat, A., Alberti-Segui, C., Rebischung, C. & Philippsen, P. Heterologous HIS3 marker and GFP reporter modules for PCR-targeting in Saccharomyces cerevisiae. Yeast 13, 1065-1075 (1997).

25. Fromant, M., Blanquet, S. & Plateau, P. Direct random mutagenesis of gene-sized DNA fragments using polymerase chain-reaction. Analytical Biochemistry 224, 347-353 (1995).

26. Sprague, G. F. Assay of yeast mating reaction. Methods In Enzymol 194, 77-93 (1991).

27. Adams, A. E. & Pringle, J. R. Staining of actin with fluorochrorme-conjugated phalloidin. Methods Enzymol 194, 729-31 (1991).

28. Pringle, J. R Staining of bud scars and other cell wall chitin vitl calcofluor. Methods Enzymol 194, 732-5 (1991).

29. Langle-Ronault, F. & Jacobs, E. A method for perfoming precise alterations in the yeast genome using a recyclable selectable marker. Nucleic Acids Research 23, 3079-3081 (1995).

30. James, P., Halladay, J. & Craig, E.A. Gienoric libraries and a host strain designed for highly efficient two-hybrid selection in yeast. Genetics 144, 1425-1436 (1996).

31. Chang, E. C., Barr, M., Wang, Y., Jung, V., Xu, H. P., and Wigler, M. H. 1994. Cooperative interaction of S. pombe proteins required for mnating and morphogenesis. Cell. 79: 131-41.

32. Pasteris, N. G., Cadle, A., Logie, L. J., Porteous, M., Schwartz, C. E., Stevenson, R. E., Glover, T. W., Wilroy, R. S., and Gorski, J. L. 1994. Isolation and characterization of the faciogenital dysplasia (aarskog-scou syndrome) gene—a putative Rho/rac guanine-nucleotide exchange factor. Cell. 79: 669-678.

33. Katzav, S., Martinzanca, D., and Barbacid, M. 1989. Vav, a novel human oncogene derived from a locus ubiquitously expressed in hematopoietic-cells. Embo Journal. 8: 2283-2290.

34. Miki, T., Smith, C. L., Long, J. E., Eva, A., and Fleming, T. P. 1993. Oncogene ect2 is related to regulators of small GTP-binding proteins. Nature. 462-465.

35. Chan, A. M., McGovern, E. S,, Catalano, G., Fleming, T. P., and Miki, T. 1994. Expression cDNA cloning of a novel oncogene with sequence similarity to regulators of snall GTP-binding proteins. Oncogene. 9: 1057-63.

36. Eva, A. and Aaronson, S. A. 1985. Isolation of a new human oncogene from a diffise B-cell lymphomna. Nature. 316: 273-275.

37. Ron, D., Tronick, S. R, Aaronson, S. A., and Eva, A. 1988. Molecular-cloning and characterization of the human dbl proto-oncogene—evidence that its overexpression is sufficient to transform nih/3t3 cells. Embo Journal. 7; 2465-2473.

38. Hart, M. J., Shanna, S., Elmasry, N., Qiu, R. G., Mccabe, P., Polakis, P., and Bollag, G. 1996. Identification of a novel guanine-nucleotide exchange factor for the Rho-gtpase. Journal Of Biological Chemistry. 271: 25452-25458.

39. Wbitehead, I., Kirk, H., Tognon, C., Trigo-Gonzalez, Ci., and Kay, P, 1995. Expression cloning of lfc, a novel oncogene with structural similarities to guanine nucleotide excinmge factors and to the regulatory region of protein kinase C. J Biol Chem. 270: 18388-95.

40. Horii, Y., Beeler, J. F., Sakaguchi, K., Tachibana, M., and Miki, T. 1994. A novel oncogene, ost, encodes a guanine nucleotide exchange factor tat potentially links Rho and Rae signaling pathways. Embo J. 13: 4776-86.

41. Glaven, J. A., Whitehead, I. P., Nomanbhoy, T., Kay, R, and Cerione, R. A. 1996. Lfc and lsc oncoproteins represent 2 new guanine-nucleotide exchange factors for the Rho-gtp-binding protein. Journal Of Biological Chemistry. 271: 27374-27381.

42. Toksoz, D. and Williams, D. A. 1994. Novel human oncogene ibc detected by transfection wiffi distinct homology regions to signal-transduction products. Oncogene. 621-628.

43. Chan, A., Takai, S., Yamada, K., and Miki, T. 1996. Isolation of a novel oncogene, netl, from neuroepithelioma cells by expression cdna cloning. Oncogene. 12: 1259-1266.

44. Hariharan, I. K. and Adams, J. M. 1987. Cdna sequence for human-ber, the gene that translocates to the abi- oncogene in chronic myeloid-leukemia. Embo Journal. 6: 115-119.

45. Diekmnami D., Brill, S., Garrett, M. D., Totty, N., Hsuan, J., Monfries, C., Hall, C., Lim, L., and Hall, A. 1991. Bcr encodes a gtpase-activating protein for p21rac. Nature. 651: 400-402.

46. Habets, G., Vanderkamrnen, R. A., Stam, J. C., Michiels, F., and Collard, J. G. 1995. Sequence of the human invasion-inducing tiarn1 gene, its conservation in evolution and its expression in tumor-cell lines of different tissue origin. Oncogene. 1371-1376.

47. Sone, M., Hoshino, M., Suzuki, E., Kuroda, S., Kaibuchi, K., Nakagoshi, H., Saigo, K., Nabeshima, Y., and Hama, C. 1997. Sti life, a protein in synaptic terminals of Drosophila homologous to GDP-GTP exchangers. Science. 275: 543-547.

48. Adams, A. E. and Pringle, J. R. (1984) Relationship of actin and tubulin distribution to bud growth in wildtype and morphogenetic inutant Saccharomyces cerevisiae. J. Cell. Biol. 98,934-945.

49. Baba, M., Baba, N., Ohsumi, Y., Kanaya, K., Osumi, M., (1989) Three dimensional analysis of morphogenesis induced by mating pheromone alpha factor in Saccharomyces cerevisiae. J Cell Sci. 94,207-216.

50. Barlow, A. J., Aldersley, T., Chattaway, F. W. (1974) Factors present in serum and seminal plasma which promote germ-tube formation and mycelial growth of Candida albicans. J. Gen. Microbiol. 82,216-272

51. Bender, A and Pringle, J. R. (1989) Multicopy suppression of the cdc24 budding defect in yeast by CDC24 and three newly identified genes including the ras-related Rsr1 (Budlp). Proc. Natl. Acad Sci. 86,9976-9980 Brown, A. J. and Gow, N. A. (1999) Regulatory networks controlling Candida albicans morphogenesis. Trends Microbiol.7,333-338.

52. Care, R. S. Trevethick, J. Binley, K. M., Sudbery, P. E. (1999) The MET3 promoter: a new tool for Candida albicans genetics. Mol. Microbiol.,34,792-798

53. Cerione, R. A. and Zheng, Y. (1996) The Dbl family of oncogenes. Curr. Opin. Cell Blol, 8,216-222

54. Chang, E. C., Barr, M., Yang, Y., Jung, V., Xu, H.-P.,Wigler, M. H. (1994) Cooperative interaction of S. pombe proteins required for mating and morphogenesis. Cell,79,131-141.

55. Clark, K. L., Feldmann, P. J., Dignard, D., Laxocque, R., Brown, A. J., Lee, M. G., Thomas, D. Y., Whiteway, M. (1995) Constitutive activation of the Saccharomyces cerevisiae mating response pathway by a MAP kinase kinase from Candida albicans. Mol. Gen. Genet. 249,609-621.

56. Csank, C., Schroppel, K., Leberer, F., Harcus, D., Moharned, O., Meloche, S., Thomas, D. Y., Whiteway, M., (1998) Roles of the Candida albicans Mitogein- activated protein kinase hornolog, Cek1p, in hyphal development and systemic Candidiasis. Infect. Immun.66,2713-2721.

57. Cutler, J. E. (1991) Putative virulence factors of Candida albicans. Ann. Rev. Microbiol.45,187-218.

58. Gimeno, C. M., Ljungdtdd, P. O., Styles, C. A., Fink, G. R. (1992) Unipolar cell divisions in the yeast Saccharomyces cerevisiae lead to filamentous growth: regulation by starvation and RAS. Cell,68,1077-1090.

59. Johnson, D. I. (1999) Cdc42: An essential Rho-type GTPase controlling eukaryotic cell polarity. Microbiol. Mol. Biol. Rev.63,54-105.

60. Kilmartin, J. V. and Adams, A. E. (1984) Stractural rearrangements of tubulin and actin during the cell cycle of the yeast Saccharomyces. J. Cell Biol. 98 ,922-933

61. Köhler, J. and Fink, G. R. (1996) Candida albicans strains heterozygous and homozygous for mutations in rnitogen-activated protein kinase signaling components have defects in hyphal development. Proc. Natl. Acad. Sci. USA,93,13223-13228

62. Leberer, E., Harcus, D., Broadbent, I. D., Clark, K. L., Dignard, D., Ziegelbauer, K. Schmidt, A. Gow, N. A., Brown, A. J. Thomas, D. Y. (1996) Signal tansduction through homologs of Ste20p and Ste7p protein kinases can trigger hyphal formation in the pathogenic flimgus Candida albicans Proc. Natl Acad. Sci. USA,93,13217-13222

63. Leng, A. Sudbery, P. E. Brown, A. J. P. (2000) Rad6p represses yeast-hyphal morphogenesis in the human fungal pathogen Candida albicans. Mol. Microbiol.,35,1264-1275.

64. Liu, H. Styles, C. A. Fink, G. R. (1993) Elements of the yeast pheromone pathway required for filamentous growth of diploids. Science, 262,1741-1744

65. Liu, H. Kohler, J. Fink, G. R. (1994) Suppression of hyphal formation in Candida albicans by mutation of a STE12 homolog. Science,266,1723-1726. Lo, H- J., Kohler, J. R, DiDomenico, B. Loebenberg, D., Cacciapuoti, A., Fin G. R. (1997) Non-filamentous C. albicans mutants are aviruent. Cell,90,939-949.

66. Miller, P. J. and Johnson, D. I. (1994) Cdc42p GTPase is involved in controlling polarised cell growth in Schizosaccharomyces pombe. Mol. Cell. Biol. 14,1075-1083

67. Mirbod F, Nakashima S, Kitajimna Y, Cannon R D, Nozawa Y (1997) Molecular cloning of a Rho family, CDC42Ca gene from Candida albicans and its mRNA expression changes during morphogenesis. J. Med Vet. Mycol,35,173-9.

68. Mösch, H. -U. Roberts, R. L. Fink C. R. (1996) Ras2 signals via the Cdc42/Ste20/mitogen-activated protein kinase module to induce filamentous growth in Saccharomnyces cerevisiae. Proc. Natl. Acad Sci. USA. 93,5352-5356

69. Murad, A. M. A., Lee, P. R., Broadbent, I. D., Barelle, C. J. Brown, A. J. P. (2000). Clp10, an efficient and convenient integration vector for Candida albicans. Yeast,16,325-327.

70. Nern, A. and Arkowitz, R. A. (1998) A GTP-exchange factor required for cell orientation. Nature,391,195-198

71. Nern, A and Arkowitz, R. A. (1999) A Cdc24p-Far1p-Gβγ protein complex required for yeast orientation during mating. J. Cell. Biol. 144,1187-1202.

72. Odds, F. C. (1988) Candia and Candidosis, 2^{nd} edn. London: Bailliere Tindall

73. Read, E. B., Okamura, H, H., Drubin, D. G., (1992) Actin and tubulin-dependant functions during S. cerevisiae mating projection formation. Mol. Biol. Cell.3,429-444.

74. Rose, M. D., Winston, F. Hieter, P. (1991) Methods in yeast genetics: A laboratory course manual. NY: Cold Spring Harbor Laboratory Press. Santos, M., Keith, G., Tuite, M. F., (1993) Non-standard translational events in Candida albicans mediated by an unusual seryl-tRNA with a 5′-CAG-3′(leucine) anticodon. EMBO J., 12,607-16

75. Sloat, B. Adams, A. Pringle, J. (1981) Roles of the CDC24 gene product in cellular morphogenesis during S. cerevisiae cell cycle. J. Cell Biol. 89,395-405 Sloat, B. F. and Pringle, J. R. (1978) A mutant of yeast defective in cellular morphogenesis Science,200,1171-3

76. Stoldt, V. R Sonnebom, A., Leuker, C. E. (1997) Efg1p, an essential regulator of morphogenesis of the human pathogen Candida albicans, is a member of a conserved class of bHLH proteins regulating morphogenetic processes in fimgi. EMBO J.6,1982-1991.

77. Whiteway, M. Dignard, D. Thomas, D. Y. (1992) Dominant negative selection of heterologous genes: isolation of Candida albicans genes that interfere with Saccharonwyces cerevisiae mating factor-induced cell cycle arrest, Proc. Natl. Acad. Sci USA, 89, 9410-9414.

78. Wilson, R. B. Davis, D., Mitchell, A. P. (1999) Rapid hypothesis testing with Candida albicans through gene disruption with shoo homology regions. J. Bact., 181,1868-1874.

79. Yaar, L. Mevarech, M. Koltin, Y. (1997) A Candida albicans RAS-related (CaRSR1) is involved in budding, cell inorphogenesis and hypha development. Microbiol., 143,1043-3044.

80. Ye. X, Szaniszlo, P. J. (2000) Expression of a constitutively active Cdc42 homologue promotes development of a sclerotic bodies but repress hyphal growth in the zoopathogenic fungus Wangiella (Exophida) dermatitidis. J Bacteriol. 182, 4941-4950

81. Zheng, Y., Bender, A., Cerione, R. A., (1995) Interactions among proteins involved in bud-site selection and bud-site assembly in Saccharomyces cerevisiae. J Biol. Chem.,270,15954-15957

1. A nucleotide sequence shown as SEQ I.D. No: 1 wherein the expression product of the nucleotide sequence has the capability of not substantially affecting the interaction of Gβ with Cdc24p or a homologue thereof that is usually capable of being associated therewith.

2. A derivative, fragment, variant or homologue of the nucleotide sequence shown as SEQ I.D. No: 1, wherein the expression product of the nucleotide sequence has the capability of not substantially affecting the interaction of Gβ with Cdc24p or a homologue thereof that is usually capable of being associated tlierewith.

3. A homologue according to claim 2 wherein the homologue comprises nucleotide residues 508 to 735 of the C. albicans Cdc24 gene presented as SEQ. I.D. No: 23.

4. A mutant of the nucleotide sequence shown as SEQ I.D. No: 1 or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

5. A method of medical treatanent comprising the step of administering a nucleotide sequence shown as SEQ I.D. No: 1 or a derivative, fragment, variant or homologue thereof.

6. A method of medical treatnent according to claim 5 whierein the homologue comprises nucleotide residues 508 to 735 of the C. albicans Cdc24 gene presented as SEQ. I.D. No: 23.

7. A method of medical treatment comprising the step of administering a mutant of the nucleotide sequence shown as SEQ I.D. No: 1 or a derivative, fragment, variant or homologue thereof or the expression product thereof.

8. A method of affecting de growth behaviour of cells comprising the step of administering the nucleotide sequence shown as SEQ I.D. No: 1 or a derivative, fragment, variant or homologue thereof or the expression product thereof to the cells.

9. A metod of affecting the growth bellaviour of cells according to claim 8, wherein the homologue comprises nucleotide residues 508 to 735 of the C. albicans Cdc24 gene presented as SEQ. I.D. No: 23.

10. A method of affecting the growth behaviour of cells comprising the step of administering a mutant of the nucleotide sequence shown as SEQ I.D. No: 1 or a derivative, fragment, variant or homologue thereof or the expression product thereof to the cells.

11. Use of a nucleotide sequence shown as SEQ I.D. No: 1 or a derivative, fragment, variant or homologue thereof or the expression product thereof in a screen to identify one or more agents that are capable of affecting the interaction of Cdc24p or a homologue thereof witlh a Gβ or an associated Rho-family GTPase.

12. The use according to claim 11, wherein the homologue comprises nucleotide residues 508 to 735 of the C. albicans Cdc24 gene presented as SEQ. I.D. No: 23

13. Use of a mutant of a nucleotide sequence shown as SEQ I.D. No: 1 or a derivative, fragment, variant or homologue thereof or the expression product thereof in a screen to identify one or more agents that are capable of affecting the interaction of Cdc24p or a homologue thereof with a Gβ or an associated Rho-family OTPase.

14. An assay comprising contacting an agent with a nucleotide sequence shown as SEQ I.D. No: 1 or a derivative, fragment, variant or homologue thereof or the expression product thereof in the presence of a Gβ capable of being associated with Cdc24p or a homologue thereof; and determining whether the agent is capable of affecting the interaction of the nueleotide sequence or the expression product with the Gβ.

15. An assay according to claim 14 wherein the homologue compriscs nucleotide residues 508 to 735 of the C. albicans Cdc24 gene presented as SEQ. I.D. No: 23.

16. An assay comprising contacting an agent with a mutant of a nucleotide sequence shown as SEQ I.D. No: 1 or a derivative, fragment, variant or homologue thereof or the expression product thereof in the presence of a Gβ capable of being associated with Cdc24p or a homologue thereof; and determining whether the agent is capable of affecting the interaction of tee mutant nucleotide sequence or the expression product with the Gβ.

17. A kit comprising a nucleotide sequence shown as SEQ. I.D No: 1 or a derivative, frament, variant or homologue thereof or the expression product thereof, and a Gβ capable of being associated with Cdc24p or a homologue thereof.

18. A kit according to claim 17 comprising a homologue of SEQ. I.D. No: 1, wherein the homologue comprises nucleotide residues 508 to 735 of the C. abicans Cdc24 gene presented as SEQ. I.D. No: 23.

19. A kit comprising a mutant of a nucleotide sequence shown as SEQ I.D. No: 1 or a derivative, fragment, variant or homologue thereof or the expression product thereof; and a Gβ capable of being associated with Cdc24p or a homologue thereof.

20. A protein sequence shown as SEQ I.D. No: 2 or a derivative, fragment, variant or homologue thereof, wherein the protein has the capability of not substantially atfecting the interaction of Gβ with Cdc24p or a homologue thereof that is usually capable of being associated with the Cdc24p or the honiologue thereof.

21. A fragment of the protein sequence shown as SEQ. I.D. No: 2 according to claim 20 wherein the fragment is the 19 amino acid Cdc24 fragment SEQ. I.D. No: 21or the 19 amino acid Dbl fragment SEQ. I.D. No: 22

22. A homologue of the protein sequence according to claim 20, wherein the homologue is the C. albicans Cdc24 76 amino acid fragment SEQ. I.D. No: 34.

23. A mutant of the protein sequence shown as SEQ I.D. No: 2 or a derivative, fagment, variant or homologue thereof, wherein the mutant protein has the capability of substantially affecting the interaction of Gβ with Cdc24p or a homnologue thereof that is usually capable of being associated with the Cdc24p or the homologue thereof.

24. The mutant according to claim 23 wherein the mnutant is the S. cerevisiae Cdc24-m1 mutant (SEQ. I.D. No: 4), the S. cerevisiae Cdc24-m2 mutant (SEQ. I.D. No: 6) and the S. cerevisiae Cdc24-m3 mutant (SEQ. I.D. No: 8)

25. A method of medical treatment comprising the step of administering a protein sequence shown as SEQ I.D. No: 2 or a derivative, fragment, variant or homnologue thereof.

26. A method according to claim 25 comprising the step of adnlinistering a fragment of the protein sequence shown as SEQ I.D. No: 2, wherein the fragment is the 19 amno acid Cdc24 fragment SEQ. I.D. No: 21.

27. A method according to claim 25 comprising the step of administering a homologue of the protein sequence shown as SEQ I.D. No: 2, wherein the homologue is the C. albicans Cdc24 76 amino acid fragment SEQ. I.D. No. 34.

28. A method of medical treatment comprising the step of administering a mutant of the protein sequence shown as SEQ I.D. No: 2 or a derivatives fragment, variant or homologue thereof for use in medicine.

29. A method according to claim 28 wherein the mutant is selected from the group comprising S. cerevisiae Cdc24-m1 76 amino acid mutant (SEQ I.D. No: 4), the S. cerevisiae Cdc24-m2 76 amino acid mutant (SEQ I.D. No: 6) and the S. cerevisiae Cdc24-m3 76 amino acid mutant (SEQ I.D. No: 8).

30. A method according to claim 28 wherein the method comprises the step of administering a fragment of a mutant of the protein sequence shown as SEQ I.D. No: 2, wherein the fragment is selected fromn the group comprising the S. cerevisiae Cdc24-m1 mutant 19 amino acid fragment (SEQ. I.D. No: 18), the S. cerevisiae Cdc24-m2 mutant 19 amino acid fragment (SEQ. I.D. No. 19) and the S. cerevisiae Cdc24-m3 nutain 19 amnino acid fragment (SEQ. I.D. No: 20).

31. A method of modulating the growth behaviour of cells comprising the step of administering a protein sequence shown as SEQ I.D. No: 2 or a derivative, fragment, variant or homologue thereof.

32. A method according to claim 31 comprising the step of administering a fragment of the protein sequence shown as SEQ I.D. No: 2, wherein the faent is the 19 amino acid S. cerevisiae Cdc24 fragment SEQ. I.D. No: 21.

33. A method according to claim 31 comprising the step of administering a homologne of the protein sequence shown as SEQ I.D. No: 2, wherein the homologue is the C. albicans Cdc24 76 amino acid fragment SEQ. I.D. No. 34.

34. A method of modulating the growth behaviour of cells comprising the step of administering a mutant of the protein sequence shown as SEQ I.D. No: 2 or a derivative, fragment, variant or homologue thereof for use in medicine.

35. A method according to claim 31 wherein the mutant is selected from the group comprising the S. cerevisiae Cdc24-m1 76 amino acid mutant (SEQ. I.D. No: 4), the S. cerevisiae Cdc24-m2 76 amino acid mutant (SEQ. I.D. No: 6) and the S. cerevisiae Cdc24-m3 76 amino acid mutant (SEQ. I.D. No: 8).

36. A method according to claim 31 wherein the method comprises the step of administerng a fragment of a mutant of the protein sequence shown as SEQ I.D. No: 2, wherein the fragment is selected from Ihe group comprising the S. cerevisiae Cdc24-m1 mutant 19 amino acid fraginent (SEQ. I.D. No: 18), the S. cerevisiae Cdc24m2 mutant 19 aino acid fragment (SEQ. I.D. No: 19) and the S. cerevisiae Cdc24m3 mutant 19 amino acid fragment (SEQ. I.D. No: 20).

37. Use of a protein sequence shown as SEQ I.D. No: 2 or a derivative, fragment, variant or homologue thereof in a screen to identify one or more agents that are capable of affecting the interaction of Cdc24p or a homologue thereof with a Gβ or an associated Rho-family GTPase.

38. The use according to claim 37 wherein a homologue of the protein sequence shown as SEQ I.D. No: 2 is used and wherein the homologue is the C. albicans Cdc24 76 amino acid fragment SEQ. I.D. No: 34

39. Use of a mutant of a protein sequence shown as SEQ I.D. No: 2 or a derivative, fragment, variant or homologue thereof in a screen to identify one or more agents that are capable of affecting the interaction of Cdc24p or a homologue thereof with a Gβ or an associated Rho-family GTPase

40. The use according to claim 39 wherein the mutant is selected fromn te group comprising the S. cerevisiae Cdc24-m1 76 amino acid mutant (SEQ. l.D. No: 4), the S. cercvisiae Cdc24-m2 76 amino acid mutant (SEQ. I.D. No: 6) and the S. cerevisiae Cdc24-m3 76 amino acid mutant (SEQ. I.D. No: 8).

41. An assay comprising contacting an agent with a protein sequence shown as SEQ I.D. No: 2 or a derivative, fragment, variant or hoinologue thereof in the presence of a Gβ capable of being associated with Cdc24p or a homologue thereof; and determining whether the agent is capable of affecting the interaction of the protein sequence with the Gβ or the Rho-family GTPase.

42. An assay according to claim 41 wherein the ag,ent is contacted with a homologue of the protein sequence shown as SEQ. I.D. No: 2, said homologue being the C. albicans Cdc24 76 arnino acid fragment SEQ. I.D. No: 34.

43. An assay comprising contacting an agent with a mutant of a protein sequence shown as SEQ I.D. No: 2or a derivative, fragment, variant or homologue thereof in the presence of Gβ capable of being associated with Cdc24p or a homologue thereof; and deterininng whether the agent is capable of affecting the interaction of the mutant protein sequence with the Gβ or the Rho-family GTPase.

44. An assay according to claim 43 wherein the mutant is selected from the group comprising S. cerevisiae Cdc24-m1 76 amino acid mutant (SEQ. I.D. No: 4), the S. cerevisiae Cdc24-m2 76 amino acid mutant (SEQ. I.D. No: 6) and the S. cerevisiae Cdc24-m3 76 amino acid mutant (SEQ. I.D. No: 8).

45. An assay according to claim 43 wherein the assay comprises contacting an agent with a fragment of a mutant of the protein sequence shown as SEQ I.D. No: 2 and wherein the fragment is selected from the group comprising the S. cerevisiae Cdc24-m1 mutant 19 amino acid fragment (SEQ. I.D. No: 18), the S. cerevisiae Cdc24-m2 mutant 19 amino acid fragment (SEQ. I.D. No: 19) and tle S. cerevisiae Cdc24-in3 mutant 19 arino acid fragment (SEQ. I.D. No: 20).

46. A kit coinprising a protein sequence shown as SEQ I.D. No.2 or a derivative, fragment, varant or homologue thereof; and a Gβ capable of being associated with Cdc24p or a homologue thereof.

47. A kit according to claim 46 wherein the kit comprises a homologue of the protein sequence shown as SEQ I.D. No: 2, said homologue being the C. albicans Cdc24 76 amino acid fragment SEQ. I.D. No: 34.

48. A kit comprising a mutant of a protein sequence shown as SEQ I.D. No: 2 or a derivative, fragment, variant or homologue thereof; and a Gβ capable of being associated with Cdc24p or a homologue thereof.

49. A kit according to claim 48 wherein the mutant is selected from the group comprising S. cerevisiae Cdc24-m1 76 amino acid mutant (SEQ. I.D. No: 4), the S. cerevisiae Cdc24-m2 76 amino acid mutant (SEQ. I.D. No: 6) and the S. cerevisiae Cdc24-m3 76 amino acid mutant (SEQ. I.D. No: 8)

50. A kit according to claim 48 wherein the kit comprises a fragment of a mutant of the protein sequence shown as SEQ I.D. No: 2 and wherein the fagment is selected from the group comprising the S. cerevisiae Cdc24-m1 mutant 19 amino acid fragment (SEQ. I.D. No: 18), the S. cerevisiae Cdc24-m2 mutant 19 amino acid fragment (SEQ. I.D. No: 19) and the S. cerevisiac Cdc24-m3 mutant 19 ailino acid fragment (SEQ. I.D. No: 20).

51. A GEF capable of interacting with a Gβ such that the interaction provides a connection between G protein coupled receptor activation and polarised cell growth.

52. An agent capable of affecting a GEF/Gβ interaction, which interaction provides a connection between G protein coupled receptor activation and polarised cell growth.

53. An assay method comprising the use of the sequence presented in SEQ ID No 4 or a nucleotide sequence coding for same

54. Use of an agent identified by the assay of any one of claims 14, 16, 41, 43 in a method of modulating cell growth.

55. A method of medical treamient according to claim 5, wherein the method is for treatment of fungal infection.

56. A method of medical treatment according to claim 6, wherein the method is for treatment of fpngal infection.

57. A method of medical treatment according to claiin 7, wherein the method is for treatment of fungal infection.

58. A method of medical treatment according to claim 25, wherein the method is for treatment of fungal infection.

59. A method of medical treatment according to claim 26, wherein the method is for treatment of fligal infection.

60. A method of medical treatment according to claimn 27, wherein the method is for treatment of fulngal infection.

61. A method of medical treatment according to claim 28, wherein the method is for treatment of fingat infection.

62. A method of medical treatment according to claim 29, wherein the method is for treatment of fungal infection.

63. A method of medical treatment according to claim 30, wherein the method is for treatment of fungal infectior.

64. A mutant of a STE4 nucleotide sequence (SEQ I.D No: 10) or a derivative, fragment, variant or homologue thereof, wherein the expression product of the mutant nucleotide sequence has the capability of substantially affecting the interaction of Gβ with Cdc24p or a homologue thereof that is usually capable of being associated therewith.

65. The mutant, derivative, fragment, variant or homologue thereof according to claim 64, wherein the mutant is SEQ. I.D. No: 12 or SEQ. I.D. No: 14.
